(19) United States
(12) Patent Application Publication
(10) Pub. No.: US 2008/0153850 A1
(43) Pub. Date: Jun. 26, 2008

(54) Adamantyl Derivates as P2x7 Receptor Antagonists

(75) Inventors: Rhonan Ford, Leicestershire (GB); Barrie Martin, Leicestershire (GB); Toby Thompson, Leicestershire (GB); Nicholas Tomkinson, Leicestershire (GB); Paul Willis, Leicestershire (GB)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 11/574,343

(22) PCT Filed: Aug. 29, 2005
(86) PCT No.: PCT/SE05/01251

§ 371 Date: Feb. 27, 2007

(30) Foreign Application Priority Data
Aug. 30, 2004 (SE) 0402103-6
Dec. 15, 2004 (SE) 0403054-0
Apr. 6, 2005 (SE) 0500766-1

(51) Int. Cl. A61K 31/195; C07C 61/135; A61K 31/4965; C07D 241/00; C07D 211/68; A61K 31/44; C07D 403/04; C07D 401/04; C07C 229/02; A61K 31/38; C07D 333/38; C07D 231/00; A61K 31/415; A61K 31/445; A61P 9/00

(52) U.S. Cl. 514/255.05; 562/499; 514/563; 514/255.06; 544/336; 546/285; 514/354; 544/405; 562/450; 514/448; 549/71; 548/374.1; 514/406; 514/318; 546/194

(57) ABSTRACT

The invention provides compounds of formula (I) pharmaceutically acceptable salt or solvate thereof, in which R¹, A¹, m and A are as defined in the specification; a process for their preparation; pharmaceutical compositions containing them; and their use in therapy.

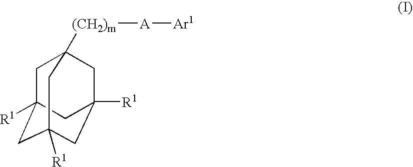

The present invention relates to adamantyl derivatives, a process for their preparation, pharmaceutical compositions containing them, a process for preparing pharmaceutical compositions and their use in therapy.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to make compounds effective as P2X₇ receptor antagonists for use in the treatment of inflammatory, immune or cardiovascular diseases, in the aetiologies of which the P2X₇ receptor may play a role.

The present invention provides a new class of adamantyl-containing P2X₇ antagonist that comprise a substituted biaromatic group. These novel compounds display excellent properties for use as P2X₇ receptor antagonists in the treatment of inflammatory, immune or cardiovascular diseases. Whilst adamantyl-containing P2X₇ antagonists have been described previously, for example in WO 00/61569, WO 03/080579 and WO 03/042190, prior to the present invention there had been no suggestion that compounds comprising the substituted biaromatic group of the present invention would make good P2X₇ antagonists. US patent application 2003/0134885 A1 concerns substituted biphenyl ligand activators of PPARgamma receptors. It does not mention the P2X₇ receptor or describe any adamantyl derivatives.

In accordance with the present invention, there is therefore provided a compound of

general formula (I), or a pharmaceutically acceptable salt or solvate thereof,

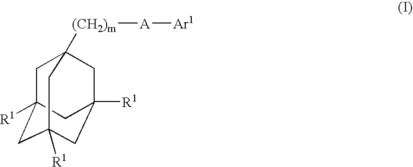

wherein m represents 1, 2 or 3;

each R¹ independently represents a hydrogen atom or a halogen;

A represents C(O)NH or NHC(O);

Ar¹ represents a group

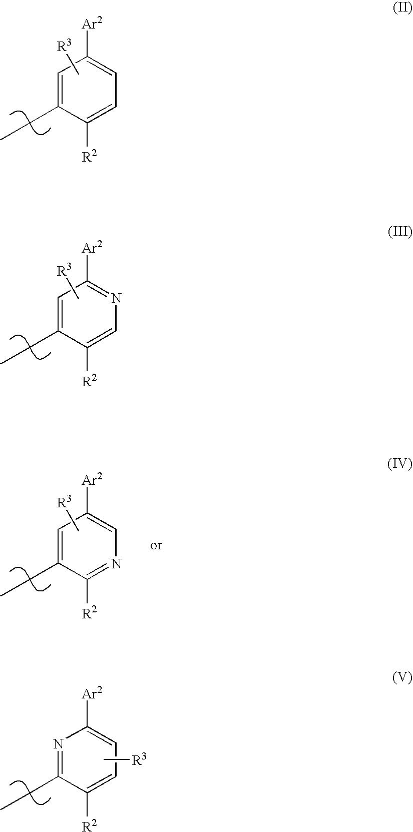

one of R² and R³ represents halogen, nitro, NR⁴R⁵, hydroxyl , or a group selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;

R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;

Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁₋₆ alkylCO₂R⁷, C₁₋₆ alkylsulphonylaminocarbonyl, NHR⁸, R⁹, XR¹⁰, C(O)NHOH and NR²⁸R²⁹;

and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;

R⁶ and R⁷ each independently represent a hydrogen atom or a C₁-C₆ alkyl group;

R⁸ represents CN, C₁-C₆ alkylsulphonyl, C₁-C₆ alkylcarbonyl, C₁-C₆ alkoxycarbonyl, C₁-C₆ alkylaminosulphonyl, or (di)-C₁-C₆ alkylaminosulphonyl;

R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;

M represents a bond, oxygen, S(O)_{q} or NR²³;

X represents oxygen, S(O)ₛ, NR²⁴, C₁-C₆ alkylene, O(CH₂)₁₋₆, NR²⁵(CH₂)₁₋₆, or S(O)ₜ(CH₂)₁₋₆;

p, q, s and t each independently represent 0, 1 or 2;

R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁₋₆ alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and

R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl; provided that:
when m is 1 and Ar¹ is a group (II) and Ar² is phenyl substituted by XR¹⁰ in a position para to Ar¹ and X is CH₂, then R¹⁰ is not a 2,4-dioxothiazolyl group, and
when m is 1 and Ar¹ is a group (II) and Ar² is phenyl substituted by MC₁₋₆ alkylCO₂R⁷ in a position para to Ar¹, then M does not represent a bond.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

In the context of the present specification, unless otherwise indicated, a “Heterocyclic” ring is an unsaturated, saturated or partially saturated ring, at least one atom of which is a heteroatom selected from oxygen, sulphur or nitrogen, and may have aliphatic or aromatic properties. “Heteroaromatic” denotes aromatic rings, at least one atom of which is a heteroatom selected from oxygen, sulphur or nitrogen. A “Carbocyclic” ring is an unsaturated, saturated or partially saturated ring, containing only carbon ring atoms, and may have aliphatic or aromatic properties. The term “Cycloalkyl” denotes saturated alkyl rings. Unless otherwise indicated an alkyl group may be linear or branched. Where a ring or group is described as being optionally substituted with at least one substituent the ring or group may be unsubstituted, or alternatively the ring or group may be substituted with, for example, one, two or three substituents.

In an embodiment of the invention, m represents 1. In another embodiment of the invention, m represents 2.

In an embodiment of the invention, each R¹ independently represents a hydrogen atom.

In an embodiment of the invention, A represents NHC(O).

In an embodiment of the invention, Ar¹ represents a group (II) or (III).

One of R² and R³ represents halogen (e.g. fluorine, chlorine, bromine or iodine), nitro, NR⁴R⁵, hydroxyl, or a group selected from (i) C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one (e.g. zero, one, two or three) halogen (e.g. fluorine, chlorine, bromine or iodine), and (ii) C₁-C₆, preferably C₁-C₄, alkoxy (e.g. methoxy, ethoxy, n-propoxy, n-butoxy, n-pentoxy or n-hexoxy) optionally substituted by at least one (e.g. zero, one, two or three) halogen (e.g. fluorine, chlorine, bromine or iodine), and the other of R² and R³ represents a hydrogen atom, halogen (e.g. fluorine, chlorine, bromine or iodine) or a C₁-C₆, preferably C₁-C₄, alkyl (e.g. methyl, ethyl, n-propyl, isopropyl, n-butyl, isobutyl, tert-butyl, n-pentyl or n-hexyl) group optionally substituted by at least one (e.g. zero, one, two or three) halogen (e.g. fluorine, chlorine, bromine or iodine).

In an embodiment of the invention, R² represents halogen, nitro, NH₂, hydroxyl, or a C₁-C₆ alkyl optionally substituted by one to three halogen substituents; and R³ represents a hydrogen atom.

In an embodiment of the invention, Ar¹ represents a group

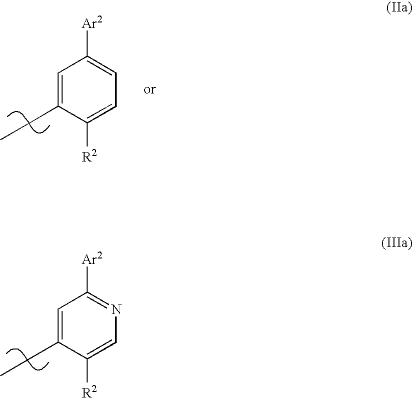

wherein R² represents halogen (e.g. fluorine, chlorine, bromine or iodine), nitro, NH₂, hydroxyl, or a C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one (e.g. zero, one, two or three) halogen (e.g. fluorine, chlorine, bromine or iodine).

R⁴ and R⁵ each independently represent a hydrogen atom or a group selected from C₁-C₆, preferably C₁-C₄, alkyl (e.g. methyl, ethyl, n-propyl, isopropyl, n-butyl, isobutyl, tert-butyl, n-pentyl or n-hexyl) and C₁-C₆, preferably C₁-C₄, alkoxy (e.g. methoxy, ethoxy, n-propoxy, n-butoxy, n-pentoxy or n-hexoxy), which C₁-C₆ alkyl or C₁-C₆ alkoxy group can be optionally substituted with at least one (e.g. zero, one, two or three) substituent selected from halogen (e.g. fluorine, chlorine, bromine or iodine), and hydroxyl.

Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one (e.g. one or two) substituent selected from CO₂R⁶, MC₁₋C₆ alkylCO₂R⁷, C₁₋C₆ alkylsulphonylaminocarbonyl (e.g MeSO₂NHCO—, or EtSO₂NHCO—), NHR⁸, R⁹, XR¹⁰, C(O)NHOH and NR²⁸R²⁹;

and which phenyl or heteroaromatic ring can further be optionally substituted by at least one (e.g. zero, one or two) substituent selected from halogen (e.g. fluorine, chlorine,

bromine or iodine), nitro, NR¹¹R¹², hydroxyl, S(O)ₚR¹³, a C₁-C₆, preferably C₁-C₄, alkoxy (e.g. methoxy, ethoxy, n-propoxy, n-butoxy, n-pentoxy or n-hexoxy) group which C₁-C₆ alkoxy group can be optionally substituted by at least one halogen (e.g. fluorine,

chlorine, bromine or iodine), and a C₁-C₆, preferably C₄-C₄, alkyl (e.g. methyl, ethyl, n-propyl, isopropyl, n-butyl, isobutyl, tert-butyl, n-pentyl or n-hexyl) group which C₁-C₆ alkyl group can be optionally substituted by at least one (e.g. zero, one or two) substituent selected from halogen (e.g. fluorine, chlorine, bromine or iodine), hydroxyl, NR¹⁴R¹⁵, SO₂NR¹⁶R¹⁷, NR¹⁸SO₂R¹⁹, NHCOR²⁰ and CONR²¹R²².

When Ar² represents phenyl or a 6-membered heteroaromatic ring, the at least one substituent selected from CO₂R⁶, MC₁₋C₆ alkylCO₂R⁷, C₁₋₆ alkylsulphonylaminocarbonyl, NHR⁸, R⁹, XR¹⁰, C(O)NHOH and NR²⁸R²⁹ may be positioned in an ortho, meta or para position relative to the bond between Ar¹ and Ar². In an embodiment of the invention, when Ar² represents phenyl or a 6-membered heteroaromatic ring the at least one substituent is in an ortho position relative to the bond between Ar¹ and Ar². In another embodiment of the invention, the at least one substituent is in a meta position relative to the bond between Ar¹ and Ar².

Examples of 5- or 6-membered heteroaromatic rings that Ar² may represent include pyrrolyl, thienyl, furanyl, imidazolyl, thiazolyl, oxazolyl, pyrazolyl, isothiazolyl, isoxazolyl, pyridyl, pyridazinyl, pyrimidinyl and pyrazinyl.

In an embodiment of the invention, Ar² represents phenyl, thienyl or a 5- or 6-membered heteroaromatic ring comprising from 1 to 2 nitrogen atoms. In a further embodiment of the invention, Ar² represents phenyl, thienyl or a 6-membered heteroaromatic ring comprising from 1 to 2 nitrogen atoms, e.g. pyridyl, pyridazinyl, pyrimidinyl or pyrazinyl. In another embodiment of the invention, Ar² represents phenyl, thienyl, pyridyl, pyrazolyl or pyrazinyl. In another embodiment of the invention Ar² represents phenyl or pyridyl.

In an embodiment of the invention, Ar² is substituted by at least one (e.g. one or two) substituent selected from carboxyl, —C₁₋C₆alkylCO₂H, —OC₁₋C₆alkylCO₂H, —NHC₁₋C₆alkylCO₂H, —N(C₁₋C₄alkyl)C₁₋C₆alkylCO₂H, —NHCN, —NHCOC₁₋C₆alkyl, —NHSO₂C₁₋C₆alkyl, —CONHSO₂C₁₋C₆alkyl, tetrazolyl and —OC₁₋C₆ alkyltetrazolyl. In a further aspect of this embodiment Ar² can further be optionally substituted by at least one substituent (e.g. zero, one or two) selected from halogen, trifluoromethyl, C₁₋C₆alkoxy and a C₁₋C₆alkyl group.

In another embodiment of the invention, Ar² is substituted by a group NR²⁸R²⁹, wherein R²⁸ and R²⁹ together with the nitrogen atom to which they are attached form a saturated heterocyclic group selected from azetidinyl, pyrrolidinyl or piperidinyl, which heterocyclic group is substituted by carboxyl and can further be optionally substituted by hydroxyl.

In a further embodiment of the invention Ar² is substituted by a substituent selected from carboxyl, MC₁₋C₆ alkylCO₂R⁷ and C₁₋₆ alkylsulphonylaminocarbonyl, and can further be optionally substituted by at least one substituent selected from halogen and a C₁₋₆alkyl group.

In another embodiment of the invention Ar² is substituted by carboxyl, and optionally at least one (e.g. zero, one or two) further substituent selected from halogen and a C₁₋C₆alkyl group.

R⁶ and R⁷ each independently represent a hydrogen atom or a C₁-C₆, preferably C₁-C₄, alkyl (e.g. methyl, ethyl, n-propyl, isopropyl, n-butyl, isobutyl, tert-butyl, n-pentyl or n-hexyl) group. In an embodiment of the invention, R⁶ and R⁷ each independently represent a hydrogen atom.

R⁸ represents CN, C₁₋C₆, preferably C₁₋C₄, alkylsulphonyl (e.g. MeSO₂— or EtSO₂—), C₁₋C₆, preferably C₁₋C₄, alkylcarbonyl (e.g. methyl-, ethyl-, n-propyl-, n-butyl-, n-pentyl- or n-hexylcarbonyl), C₁₋C₆, preferably C₁₋C₄, alkoxycarbonyl (e.g. methoxy-, ethoxy-, n-propoxy-, n-butoxy-, n-pentoxy- or n-hexoxycarbonyl), C₁₋C₆, preferably C₁-C₄, alkylaminosulphonyl (e.g. MeNHSO₂ or EtNHSO₂—), or (di)-C₁₋C₆, preferably C₁₋C₄, alkylaminosulphonyl (e.g. Me₂NSO₂ or Et₂NSO₂— or EtMeNSO₂—).

R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one (e.g. one, two or three) substituent selected from hydroxyl, ═O and ═S.

In an embodiment of the invention R⁹ and R¹⁰ each independently represent tetrazolyl.

When R⁹ and R¹⁰ each independently represent a 5- to 6-membered heterocyclic ring, nitrogen heteroatoms in the heterocyclic ring may carry hydroxyl substituents and sulphur atoms in the ring may be in the form of S, SO (i.e. carrying one ═O substituent) or SO₂ (i.e. carrying two ═O substituents).

Where R⁹ or R¹⁰ represents a 5- to 6-membered heterocyclic ring comprising from 1-4 heteroatoms independently selected from nitrogen, oxygen and sulphur, which heterocyclic ring is substituted by at least one substituent selected from hydroxyl, ═O and ═S, examples include:

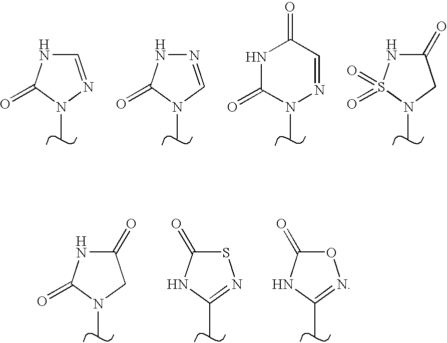

In an embodiment of the invention, M represents a bond or oxygen. In another embodiment of the invention, M represents a bond.

In an embodiment of the invention, X represents oxygen, or C₁₋₆, preferably C₁₋₄, alkylene.

In an embodiment of the invention, p, q, s and t each independently represent 2. In another embodiment of the invention, p, q, s and t each independently represent 0.

In an embodiment of the invention, where Ar¹ is a group (V) and Ar² is a thiazolyl group substituted by NH₂ and NHR⁸, R⁸ is not C₁₋C₆ alkylcarbonyl or C₁₋C₆ alkoxycarbonyl. R²⁸ and R²⁹ together with the nitrogen atom to which they are attached form a 3- to 8-membered saturated heterocyclic ring, which heterocyclic ring is substituted with at least one substituent (e.g. one, two or three) independently selected from CO₂R⁶, MC₁₋₆ alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent (e.g. zero, one, two or three) independently selected from hydroxyl, halogen (e.g. fluorine, chlorine, bromine or iodine), C₁-C₆ alkoxy (e.g. methoxy, ethoxy, n-propoxy, n-butoxy, n-pentoxy or n-hexoxy) optionally substituted by at least one halogen, and a C₁-C₆ alkyl group (e.g. methyl, ethyl, n-propyl, isopropyl, n-butyl, isobutyl, tert-butyl, n-pentyl or n-hexyl) which C₁-C₆ alkyl group can be optionally substituted (e.g. zero, one, two or three) by at least one substituent independently selected from halogen and hydroxyl. Examples of saturated heterocyclic rings that R²⁸ and R²⁹ together with the nitrogen atom to which they are attached may form are rings containing one or two nitrogen atoms, e.g. pyrrolidinyl, piperidinyl, piperazinyl, homopiperazinyl, homopiperidinyl and azetidinyl.

R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆, preferably C₁-C₄, alkyl (e.g. methyl, ethyl, n-propyl, isopropyl, n-butyl, isobutyl, tert-butyl, n-pentyl or n-hexyl) and C₁-C₆, preferably C₁-C₄, alkoxy (e.g. methoxy, ethoxy, n-propoxy, n-butoxy, n-pentoxy or n-hexoxy), which C₁-C₆ alkyl or C₁-C₆ alkoxy group can be optionally substituted with at least one (e.g. zero, one, two or three) substituent selected from halogen (e.g. fluorine, chlorine, bromine or iodine) and hydroxyl.

In a further aspect of the invention, there is provided a compound of general formula (IB), or a pharmaceutically acceptable salt or solvate thereof,

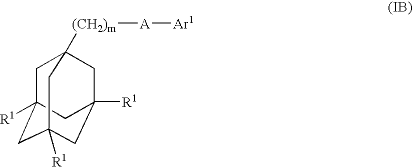

wherein m represents 1, 2 or 3;

each R¹ independently represents a hydrogen atom or a halogen;

A represents C(O)NH or NHC(O);

Ar¹ represents a group

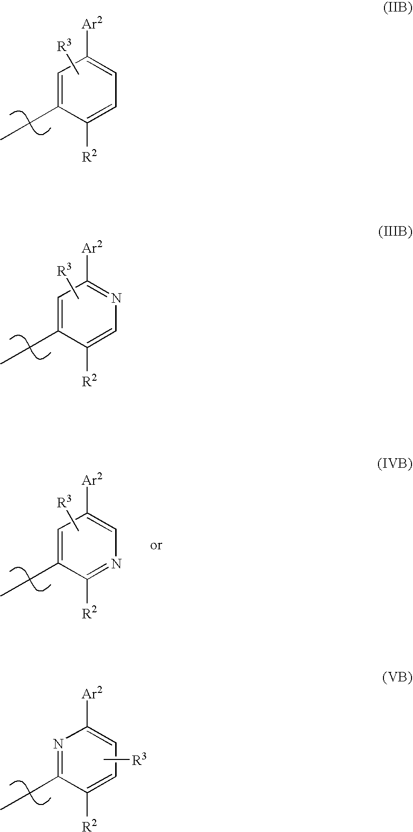

one of R² and R³ represents halogen, nitro, NR⁴R⁵, hydroxyl, or a group

selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;

R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;

Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁₋₆ alkylCO₂R⁷, C₁₋₆ alkylsulphonylaminocarbonyl, NHR⁸, R⁹ and XR¹⁰,

and which phenyl or heteroaromatic ring can further be optionally substituted by at least

one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;

R⁶ and R⁷ each independently represent a hydrogen atom or a C₁₋C₆ alkyl group;

R⁸ represents CN, C₁₋C₆ alkylsulphonyl, C₁₋C₆alkylcarbonyl, C₁₋C₆ alkoxycarbonyl, C₁₋C₆ alkylaminosulphonyl, or (di)-C₁-C₆ alkylaminosulphonyl;

R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;

M represents a bond, oxygen, S(O)_{q} or NR²³;

X represents oxygen, S(O)ₛ, NR²⁴, C₁₋C₆ alkylene, O(CH₂)₁₋₆, NR²⁵(CH₂)₁₋₆, or

S(O)ₜ(CH)₁₋₆;

p, q, s and t each independently represent 0, 1 or 2; and

R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;

provided that when m is 1 and Ar¹ is a group (II) and Ar² is phenyl substituted by XR¹⁰ in a position para to Ar¹ and X is CH₂, then R¹⁰ is not a 2,4-dioxothiazolyl group; and when m is 1 and Ar¹ is a group (II) and Ar² is phenyl substituted by MC₁₋C₆ alkylCO₂R⁷ in a position para to Ar¹, then M does not represent a bond.

In a still further aspect of the present invention there is provided a compound of formula (IC), or a pharmaceutically acceptable salt or solvate thereof,

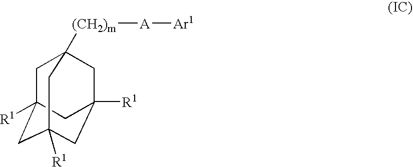

wherein m represents 1,2 or 3;

each R¹ independently represents a hydrogen atom;

A represents C(O)NH or NHC(O);

Ar¹ represents a group

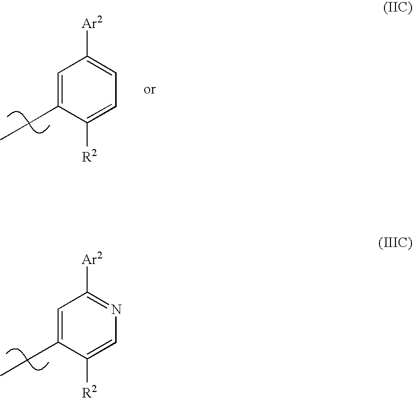

wherein R² represents halogen, nitro, NH₂, hydroxyl or a C₁-C₆ alkyl optionally substituted by one to three halogen atoms;

Ar² represents phenyl, thienyl or a 5- or 6-membered heteroaromatic ring comprising from 1 to 2 nitrogen atoms, wherein Ar² is substituted by at least one substituent selected from carboxyl, —C₁₋C₆alkylCO₂H, —OC₁₋C₆alkylCO₂H, —NHC₁₋C₆alkylCO₂H, —N(C₁₋C₄alkyl)C₁₋C₆alkylCO₂H, —NHCN, —NHCOC₁₋C₆alkyl, —NHSO₂C₁₋C₆alkyl, —CONHSO₂C₁₋C₆alkyl, tetrazolyl, —OC₁₋C₆ alkyltetrazolyl and NR²⁸R²⁹, and wherein Ar² can further be optionally substituted by at least one substituent selected from halogen, trifluoromethyl, C₁₋₆alkoxy and a C₁₋₆alkyl group; and

R²⁸ and R²⁹ together with the nitrogen atom to which they are attached form a saturated heterocyclic group selected from azetidinyl, pyrrolidinyl or piperidinyl, which heterocyclic group is substituted by carboxyl and can further be optionally substituted by hydroxyl; provided that when Ar¹ is a group (IIC) and Ar² is phenyl substituted by C₁₋C₆ alkylCO₂H in a position para to Ar¹, then m is not 1.

In an embodiment of the invention, the compound of formula (I) is selected from
4′-Chloro-3′-[[(tricyclo[3.3.1.1^{3,7}]dec-1-ylmethyl)amino]carbonyl]-[1,1′-biphenyl]-4-carboxylic acid,
4′-Chloro-3′-[[(tricyclo[3.3.1.1^{3,7}]dec-1-ylmethyl)amino]carbonyl]-[1,1′-biphenyl]-3-carboxylic acid,
4′-Chloro-3′-[[(tricyclo[3.3.1.1^{3,7}]dec-1-ylmethyl)amino]carbonyl]-[1,1′-biphenyl]-2-carboxylic acid,
2-Chloro-5-[6-(cyanoamino)pyrazinyl]-N-(tricyclo[3.3.1.1^{3,7}]dec-1-ylmethyl)-benzamide,
2-Chloro-5-[3-(cyanamino)pyrazinyl]-N-(tricyclo[3.3.1.1^{3,7}]dec-1-ylmethyl)-benzamide,
3-[4-Chloro-3-[[(tricyclo[3.3.1.1^{3,7}]dec-1-ylmethyl)amino]carbonyl]phenyl]-2-pyrazinecarboxylic acid,
3-[5-Chloro-4-[[(tricyclo [3.3.1.1^{3,7}]dec-1-ylmethyl)amino]carbonyl]-2-pyridinyl]-benzoic acid,
2-Chloro-5-[3-[(methylsulfonyl)amino)pyrazinyl]-N-(tricyclo[3.3.1.1^{3,7}]dec-1-ylmethyl)-benzamide,
2-Chloro-5-[3-(1-H-tetrazol-5-yl)pyrazinyl]-N-(tricyclo[3.3.1.1^{3,7}]dec-1-ylmethyl)-benzamide,
2-[4-Chloro-3-[[(tricyclo[3.3.1.1^{3,7}]dec-1-ylmethyl)amino]carbonyl]phenyl]-3-pyridinecarboxylic acid,
5-[4-Chloro-3-[[(tricyclo[3.3.1.1^{3,7}]dec-1-ylmethyl)amino]carbonyl]phenyl]-3-pyridinecarboxylic acid,
2-[4-Chloro-3-[[(tricyclo[3.3.1.1^{3,7}]dec-1-ylmethyl)amino]carbonyl]phenyl]-4-pyridinecarboxylic acid,
2-[4-Chloro-3-[[(tricyclo[3.3.1.1^{3,7}]dec-1-ylmethyl)amino]carbonyl]phenyl]-6-methyl-3-pyridinecarboxylic acid,
(2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid,
[[4′-Chloro-3′-[[(tricyclo[3.3.1.1^{3,7}]dec-1-ylmethyl)amino]carbonyl][1,1′-biphenyl]-2-yl]oxy]-acetic acid,
3-[[4′-Chloro-3′-[[(tricyclo[3.3.1.1^{3,7}]dec-1-ylmethyl)amino]carbonyl][1,1′-biphenyl]-2-yl]oxy]-propanoic acid,
5-Chloro-2-[4-chloro-3-[[(tricyclo[3.3.1.1^{3,7}]dec-1-ylmethyl)amino]carbonyl]phenyl]-3-pyridinecarboxylic acid,
4′-Chloro-6-methyl-3′-[[(tricyclo[3.3.1.1^{3,7}]dec-1-ylmethyl)amino]carbonyl]-[1,1′-biphenyl]-2-carboxylic acid,
3-[4-Chloro-3-[[(tricyclo[3.3.1.1^{3,7}]dec-1-ylmethyl)amino]carbonyl]phenyl]-2-thiophenecarboxylic acid,
6-[4-Chloro-3-[[(tricyclo[3.3.1.1^{3,7}]dec-1-ylmethyl)amino]carbonyl]phenyl]-2-pyridinecarboxylic acid,
3-[4-Chloro-3-[[(tricyclo[3.3.1.1^{3,7}]dec-1-ylmethyl)amino]carbonyl]phenyl]-2-pyridinecarboxylic acid,
2-Chloro-5-[2-(1H-tetrazol-5-yl)-3-pyridinyl]-N-(tricyclo[3.3.1.1^{3,7}]dec-1-ylmethyl)-benzamide,
2-[4-Chloro-3-[[(tricyclo[3.3.1.1^{3,7}]dec-1-ylmethyl)amino]carbonyl]phenyl]-4-oxazolecarboxylic acid,
4′-Chloro-4-methyl-3′-[[(tricyclo[3.3.1.1^{3,7}]dec-1-ylmethyl)amino]carbonyl]-[1,1′-biphenyl]-2-carboxylic acid,
6-[4-Chloro-3-[[(tricyclo[3.3.1.1^{3,7}]dec-1-ylmethyl)amino]carbonyl]phenyl]-N-(methylsulfonyl)-2-pyridinecarboxamide,
N-[3-[4-Chloro-3-[[(tricyclo[3.3.1.1^{3,7}]dec-1-ylmethyl)amino]carbonyl]phenyl]-2-pyridinyl]-glycine,
2-Chloro-5-[6-[(methylsulfonyl)amino]-2-pyridinyl]-N-(tricyclo[3.3.1.1^{3,7}]dec-1-ylmethyl)-benzamide,
[[3-[4-Chloro-3-[[(tricyclo[3.3.1.1^{3,7}]dec-1-ylmethyl)amino]carbonyl]phenyl]-2-pyridinyl]oxy]-acetic acid,
2-Chloro-5-[3-(1H-tetrazol-5-ylmethoxy)-2-pyridinyl]-N-(tricyclo[3.3.1.1^{3,7}]dec-1-ylmethyl)-benzamide,
4′-Chloro-4-methoxy-3′-[[(tricyclo[3.3.1.1^{3,7}]dec-1-ylmethyl)amino]carbonyl]-[1,1′-biphenyl]-2-carboxylic acid,
4-[4-Chloro-3-[[(tricyclo [3.3.1.1^{3,7}]dec-1-ylmethyl)amino]carbonyl]phenyl]-1-methyl-1H-pyrazole-3-carboxylic acid,
4-[4-Chloro-3-[[(tricyclo[3.3.1.1^{3,7}]dec-1-ylmethyl)amino]carbonyl]phenyl]-1-methyl-1H-pyrazole-5-carboxylic acid,
N-[3-[4-Chloro-3-[[(tricyclo[3.3.1.1^{3,7}]dec-1-ylmethyl)amino]carbonyl]phenyl]pyrazinyl]-N-methyl-glycine,
1-[3-[4-Chloro-3-[[(tricyclo[3.3.1.1^{3,7}]dec-1-ylmethyl)amino]carbonyl]phenyl]pyrazinyl]-4-piperidinecarboxylic acid,
4′-Chloro-6-fluoro-3′-[[(tricyclo[3.3.1.1^{3,7}]dec-1-ylmethyl)amino]carbonyl]-[1,1′-biphenyl]-2-carboxylic acid,
4′-Chloro-5-fluoro-3′-[[(tricyclo[3.3.1.1^{3,7}]dec-1-ylmethyl)amino]carbonyl]-[1,1′-biphenyl]-2-carboxylic acid,
4′-Chloro-3′-[[(tricyclo[3.3.1.1^{3,7}]dec-1-ylmethyl)amino]carbonyl]-[1,1′-biphenyl]-2-acetic acid,
[[4′-Chloro-3′-[[(tricyclo[3.3.1.1^{3,7}]dec-1-ylmethyl)amino]carbonyl][1,1′-biphenyl]-3-yl]oxy]-acetic acid,
(2R)-2-[[4′-Chloro-3′-[[(tricyclo[3.3.1.1^{3,7}]dec-1-ylmethyl)amino]carbonyl][1,1′-biphenyl]-2-yl]oxy]-propanoic acid,
[[4′-Chloro-3′-[[(tricyclo[3.3.1.1^{3,7}]dec-1-ylmethyl)amino]carbonyl][1,1′-biphenyl]-4-yl]oxy]-acetic acid,
(2S)-2-[[4′-Chloro-3′-[[(tricyclo[3.3.1.1^{3,7}]dec-1-ylmethyl)amino]carbonyl][1,1-biphenyl]-3-yl]oxy ]-propanoic acid,
4,4′-Dichloro-3′-[[(tricyclo[3.3.1.1^{3,7}]dec-1-ylmethyl)amino]carbonyl]-[1,1′-biphenyl]-2-carboxylic acid,
(2S)-2-[[4′-Chloro-3′-[[(tricyclo[3.3.1.1^{3,7}]dec-1-ylmethyl)amino]carbonyl][1,1′-biphenyl]-4-yl]oxy]-propanoic acid,
3-Chloro-6-[4-chloro-3-[[(tricyclo[3.3.1.1^{3,7}]dec-1-ylmethyl)amino]carbonyl]phenyl]-2-pyridinecarboxylic acid,
3-[4-Chloro-3-[[(tricyclo[3.3.1.1^{3,7}]dec-1-ylmethyl)amino]carbonyl]phenyl]-4-pyridinecarboxylic acid,
[[2-[4-Chloro-3-[[(tricyclo[3.3.1.1^{3,7}]dec-1-ylmethyl)amino]carbonyl]phenyl]-3-pyridinyl]oxy]-acetic acid,
N-[2-[4-Chloro-3-[[(tricyclo[3.3.1.1^{3,7}]dec-1-ylmethyl)amino]carbonyl]phenyl]-3-pyridinyl]-glycine,
4′-Chloro-4,5-difluoro-3′-[[(tricyclo[3.3.1.1^{3,7}]dec-1-ylmethyl)amino]carbonyl]-[1,1′-biphenyl]-2-carboxylic acid,
4′-Chloro-3′-[[(2-tricyclo[3.3.1.1^{3,7}]dec-1-ylethyl)amino]carbonyl]-[1,1′-biphenyl]-2-carboxylic acid,
3-[4-Chloro-3-[[(2-tricyclo[3.3.1.1^{3,7}]dec-1-ylethyl)amino]carbonyl]phenyl]-2-pyridinecarboxylic acid,
4′-Chloro-4-fluoro-3′-[[(tricyclo[3.3.1.1^{3,7}]dec-1-ylmethyl)amino]carbonyl]-[1,1′-biphenyl]-2-carboxylic acid,
2-[5-Chloro-4-[[(tricyclo[3.3.1.1^{3,7}]dec-1-ylmethyl)amino]carbonyl]-2-pyridinyl]-benzoic acid,
2-[4-Chloro-3-[[(tricyclo[3.3.1.1^{3,7}]dec-1-ylmethyl)amino]carbonyl]phenyl]-4-methyl-3-pyridinecarboxylic acid,
6-[4-Chloro-3-[[(tricyclo[3.3.1.1^{3,7}]dec-1-ylmethyl)amino]carbonyl]phenyl]-2-[(2-hydroxyethyl)methylamino]-3-pyridinecarboxylic acid,
3-[4-Methyl-3-[[(tricyclo[3.3.1.1^{3,7}]dec-1-ylmethyl)amino]carbonyl]phenyl]-2-pyridinecarboxylic acid,
4-[4-Chloro-3-[[(tricyclo[3.3.1.1^{3,7}]dec-1-ylmethyl)amino]carbonyl]phenyl]-1,3-dimethyl-1H-pyrazole-5-carboxylic acid,
2-[4-Methyl-3-[[(tricyclo[3.3.1.1^{3,7}]dec-1-ylmethyl)amino]carbonyl]phenyl]-3-pyridinecarboxylic acid,
3-[4-Chloro-3-[[(tricyclo[3.3.1.1^{3,7}]dec-1-ylmethyl)amino]carbonyl]phenyl]-2-pyridineacetic acid,
1-[3-[4-Chloro-3-[[(tricyclo[3.3.1.1^{3,7}]dec-1-ylmethyl)amino]carbonyl]phenyl]-2-pyridinyl]-4-piperidinecarboxylic acid,
1-[3-[4-Chloro-3-[[(tricyclo[3.3.1.1^{3,7}]dec-1-ylmethyl)amino]carbonyl]phenyl]-2-pyridinyl]-L-proline,
1-[3-[4-Chloro-3-[[(tricyclo[3.3.1.1^{3,7}]dec-1-ylmethyl)amino]carbonyl]phenyl]-2-pyridinyl]-3-piperidinecarboxylic acid,
1-[3-[4-Chloro-3-[[(tricyclo[3.3.1.1^{3,7}]dec-1-ylmethyl)amino]carbonyl]phenyl]-2-pyridinyl]-3-azetidinecarboxylic acid,
3-[4-Chloro-3-[[(tricyclo [3.3.1.1^{3,7}]dec-1-ylmethyl)amino]carbonyl]phenyl]-6-methyl-2-pyridinecarboxylic acid,
3-[4-Chloro-3-[[(tricyclo[3.3.1.1^{3,7}]dec-1-ylmethyl)amino]carbonyl]phenyl]-5-methyl-2-pyridinecarboxylic acid,
1-[3-[4-Chloro-3-[[(tricyclo[3.3.1.1^{3,7}]dec-1-ylmethyl)amino]carbonyl]phenyl]-2-pyridinyl]-4-hydroxy-4-piperidinecarboxylic acid,
1-[3-[4-Chloro-3-[[(tricyclo[3.3.1.1^{3,7}]dec-1-ylmethyl)amino]carbonyl]phenyl]-5-fluoro-2-pyridinyl]-4-piperidinecarboxylic acid,
4′-Methyl-3′-[[(tricyclo[3.3.1.1^{3,7}]dec-1-ylmethyl)amino]carbonyl]-[1,1′-biphenyl]-2-carboxylic acid,
1-[3-[4-Methyl-3-[[(tricyclo[3.3.1.1^{3,7}]dec-1-ylmethyl)amino]carbonyl]phenyl]-2-pyridinyl]-4-piperidinecarboxylic acid,
6-[4-Methyl-3-[[(tricyclo[3.3.1.1^{3,7}]dec-1-ylmethyl)amino]carbonyl]phenyl]-2-pyridinecarboxylic acid,
4-[4-Chloro-3-[[(tricyclo[3.3.1.1^{3,7}]dec-1-ylmethyl)amino]carbonyl]phenyl]-3-pyridinecarboxylic acid,
6-[4-Chloro-3-[[(tricyclo[3.3.1.1^{3,7}]dec-1-ylmethyl)amino]carbonyl]phenyl]-3-methyl-2-pyridinecarboxylic acid,
6-[4-Chloro-3-[[(tricyclo[3.3.1.1^{3,7}]dec-1-ylmethyl)amino]carbonyl]phenyl]-4-(trifluoromethyl)-2-pyridinecarboxylic acid, or
5-[6-(Acetylamino)-2-methyl-3-pyridinyl]-2-chloro-N-(tricyclo[3.3.1.1^{3,7}]dec-1-ylmethyl)-benzamide
   or a pharmaceutically acceptable salt or solvate thereof.

The present invention further provides a process for the preparation of a compound of formula (I) as defined above, or a pharmaceutically acceptable salt or solvate thereof, which comprises

(a) reacting a compound of formula

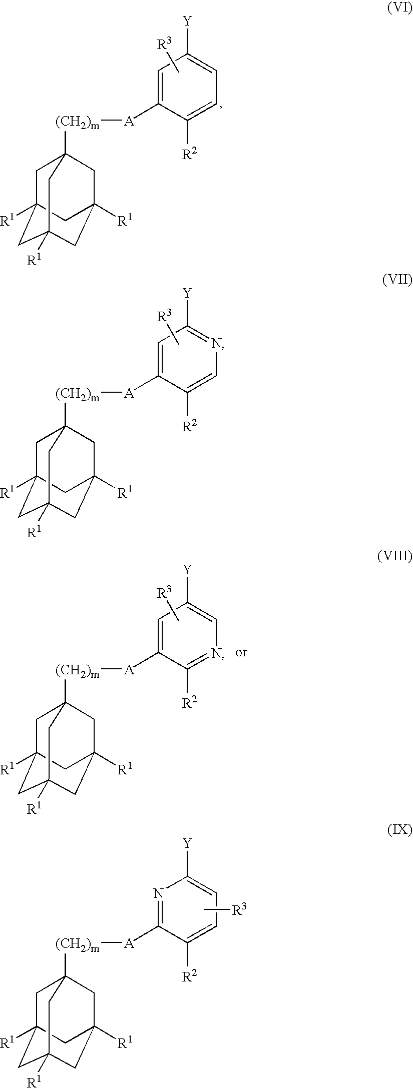

with a compound of formula

Z-Ar² (X)

wherein one of Y and Z represents a displaceable group such as a metallic, organometallic or organosilicon group (e.g. copper, lithium, an organoboron group such as B(OH)₂, B(OⁱPr)₂, BEt₂ or a boronic acid pinacol cyclic ester, or an organotin group such as SnMe₃ or SnBu₃, an organosilicon group such as Si(Me)F₂, an organoaluminium group such as AlEt₂, an organomagnesium group such as MgCl, MgBr or MgI, or an organozinc group such as ZnCl, ZnBr or ZnI) and the other of Y and Z represents a leaving group such as a halogeno or sulphonyloxy group (e.g. a chloro, bromo, iodo, trifluoromethanesulphonyloxy, methanesulphonyloxy or paratoluenesulphonyloxy group) and R¹, m, A, Ar², R² and R³ are as defined for formula (I); or

(b) when Ar² is substituted by carboxyl, reacting a compound of formula (VI)-(IX) as defined in (a) above with a compound of formula

Z-Ar^{2a}—CN (XI)

wherein Z is as defined in formula (X), and Ar^{2a} represents a phenyl or 5- or 6-membered heteroaromatic ring comprising from 1 to 2 heteroatoms independently selected from nitrogen, oxygen and sulphur, followed by reaction with a base such as sodium hydroxide or lithium hydroxide in a solvent such as water, acetonitrile or methanol, at a temperature in the range 0-150° C., optionally followed by reaction with an acid such as hydrochloric acid in a solvent such as water, at a temperature in the range 0-150° C.; or

(c) when R⁹ represents tetrazolyl, reacting a compound of formula (VI)-(IX) as defined in (a) above with a compound of formula (XI) as defined in (b) above, followed by reaction with a suitable source of azide (e.g. sodium azide, ammonium azide, azidotrimethylsilane or azidotributyltin); or

(d) when R⁸ represents CN, C₁₋₆ alkylsulphonyl, C₁₋₆ alkylcarbonyl, C₁₋₆ alkoxycarbonyl, C₁₋₆ alkylaminosulphonyl, or (di)-C₁₋₆ alkylaminosulphonyl, reacting a compound of formula (VI)-(IX) as defined in (a) above with a compound of formula

L¹-Ar^{2b}-Z (XII)

wherein L¹ represents a leaving group such as a halogeno or sulphonyloxy group (e.g. a chloro, bromo, iodo, trifluoromethanesulphonyloxy, methanesulphonyloxy or paratoluenesulphonyloxy group), Ar^{2b} represents phenyl or a 5- or 6-membered heteroaromatic ring comprising from 1 to 2 heteroatoms independently selected from nitrogen, oxygen and sulphur, and Z is as defined in formula (X), followed by reaction with a compound of formula

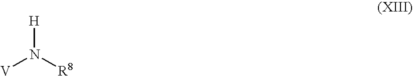

wherein V represents a hydrogen or a metallic group, for example sodium, or

(e) when Ar² is substituted by carboxyl, reacting a compound of formula (VI)-(IX) as defined in (a) above with a compound of formula (XII) as defined in (d) above, followed by reaction with a suitable source of cyanide (e.g. sodium cyanide, potassium cyanide, copper cyanide or zinc cyanide), followed by reaction with a base such as sodium hydroxide or lithium hydroxide in a solvent such as water, acetonitrile or methanol, at a temperature in the range 0-150° C., optionally followed by reaction with an acid such as hydrochloric acid in a solvent such as water, at a temperature in the range 0-150° C.; or

(f) when R⁹ represents tetrazolyl, reacting a compound of formula (VI)-(IX) as defined in (a) above with a compound of formula (XII) as defined in (d) above, followed by reaction with a suitable source of cyanide (e.g. sodium cyanide, potassium cyanide, copper cyanide or zinc cyanide), followed by reaction with a suitable source of azide (e.g. sodium azide, ammonium azide, azidotrimethylsilane or azidotributyltin); or

(g) when Ar² is substituted by carboxyl, reacting a compound of formula (VI)-(LX) as defined in (a) above with a compound of formula (XII) as defined in (d) above, followed by reaction with carbon monoxide and an alcohol in the presence of a suitable catalyst, for example a palladium catalyst, followed by reaction with a base such as sodium hydroxide or lithium hydroxide in a solvent such as water or methanol, at a temperature in the range 0-150° C.; or

(h) when Ar² represents a group of formula

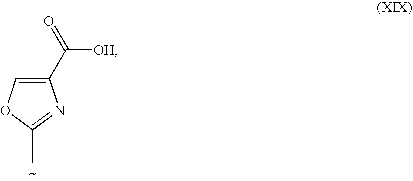

reacting a compound of formula

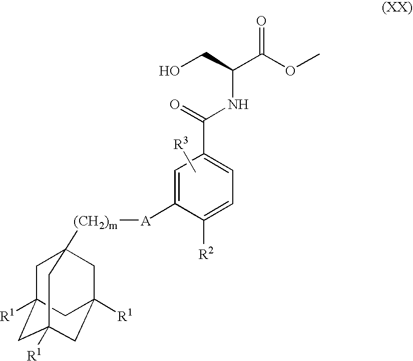

with a suitable cyclodehydrating reagent (e.g. diethylaminosulfur trifluoride), followed by reaction with a suitable oxidising reagent (e.g. bromotrichloromethane and 1,8-diazabicyclo[5.4.0]undec-7-ene), followed by reaction with a base such as sodium hydroxide or lithium hydroxide in a solvent such as water or methanol, at a temperature in the range 0-150° C.; or

(i) when M represents oxygen or NR²³, reacting a compound of formula (VI)-(IX) as defined in (a) above, with a compound of formula (XII) as defined in (d) above, followed by reaction with a compound of formula

H-M-C₁₋₆alkyl-CO₂R⁷ (XXI)

wherein M represents oxygen or NR²³, and R²³ and R⁷ are as defined in formula (I), optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or

(j) when M represents oxygen or NR²³, reacting a compound of formula (XXI) as defined in (i) above, with a compound of formula (XII) as defined in (d) above, followed by reaction with a compound of formula (VI)-(IX) as defined in (a) above, optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or

(k) when M represents oxygen or NR²³, reacting a compound of formula (VI)-(IX) as defined in (a) above, with a compound of formula

H-M-Ar^{2c}-Z (XXII)

wherein Ar^{2c} represents phenyl or a 5- or 6-membered heteroaromatic ring comprising from 1 to 2 heteroatoms independently selected from nitrogen, oxygen and sulphur, Z is as defined in formula (X), and M represents oxygen or NR²³, wherein R²³ is as defined in formula (I), followed by reaction with either β-propiolactone or a compound of formula

L¹-C₁₋₆alkyl-CO₂R⁷ (XXIII)

wherein R⁷ is as defined in formula (I), and L¹ is as defined in formula (XII), optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or

(l) when X represents O(CH₂)₁₋₆ or NR²⁵(CH₂)₁₋₆ and R¹⁰ represents tetrazolyl, reacting a compound of formula (VI)-(IX) as defined in (a) above, with a compound of formula

H-M¹-Ar^{2d}-Z (XXIV),

wherein M¹ represents oxygen or NR²⁵, R²⁵ is as defined in formula (I), Ar^{2d} represents a phenyl, 5- or 6-membered heteroaromatic ring comprising from 1 to 2 heteroatoms independently selected from nitrogen, oxygen and sulphur, and Z is as defined in formula (X), followed by reaction with a compound of formula

L¹-C₁₋₆alkyl-CN (XXV)

wherein L¹ is as defined in formula (XII), followed by reaction with a suitable source of azide (e.g. sodium azide, ammonium azide, azidotrimethylsilane or azidotributyltin);

(m) when Ar² is substituted by carboxyl, reacting a compound of formula (VI)-(IX) as defined in (a) above with a compound of formula

Z-Ar^{2e}—C(O)H (XXVIII)

wherein Z is as defined in formula (X), and Ar²e represents a phenyl or 5- or 6-membered heteroaromatic ring comprising from 1 to 2 heteroatoms independently selected from nitrogen, oxygen and sulphur, followed by reaction with an oxidising agent such as potassium peroxymonosulfate or sodium chlorite in a solvent such as N,N-dimethylformamide at a temperature in the range 0-100° C.; or

(n) reacting a compound of formula

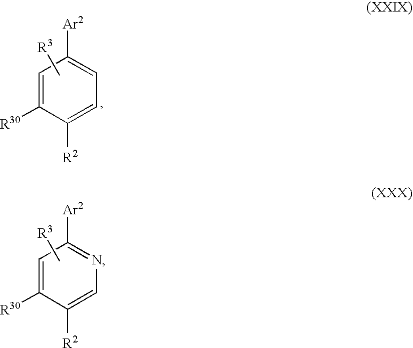

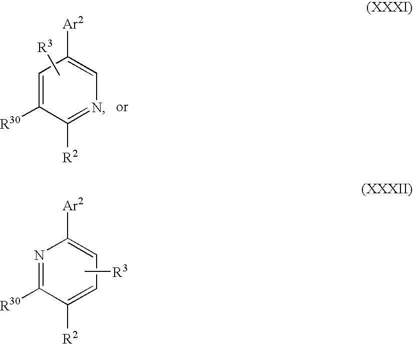

with a compound of formula

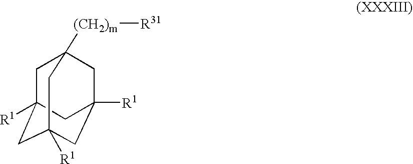

wherein one of R³⁰ and R³¹ represents NH₂ and the other of R³⁰ and R³¹ represents CO₂H, COBr or COCl, and Ar², R¹, R², R³, R⁶ and m are as defined in formula (I); or

(o) when R²⁸ and R²⁹ together with the nitrogen to which they are attached form a 3- to 8-membered saturated heterocyclic ring, which heterocyclic ring is substituted by CO₂R⁶, reacting a compound of formula (VI)-(IX) as defined in (a) above, with a compound of formula (XII) as defined in (d) above, followed by reaction with a compound of formula

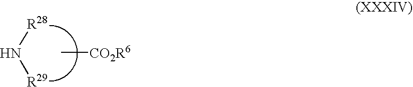

wherein R⁶, R²⁸ and R²⁹ are as defined in formula (I), optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.; or

(p) when R²⁸ and R²⁹ together with the nitrogen to which they are attached form a 3- to 8-membered saturated heterocyclic ring, which heterocyclic ring is substituted by CO₂R⁶, reacting a compound of formula (XII) as defined in (d) above with a compound of formula (XXXIV) as defined in (0) above, followed by reaction with a compound of formula (VI)-(IX) as defined in (a) above, optionally followed by reaction with a base such as sodium hydroxide or lithium hydroxide in a solvent such as water or methanol, at a temperature in the range 0-150° C., or optionally followed by reaction with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.;

and optionally after (a), (b), (c), (d), (e), (f), (g), (h), (i), (j), (k), (l), (m), (n), (o) or (p), carrying out one or more of the following:
converting the compound to a further compound of the invention
forming a pharmaceutically acceptable salt or solvate of the compound.

In formula (XI), (XII), (XXII), (XXIV) and (XXVIII) above, Ar^{2a}, Ar^{2b}, Ar^{2c} Ar^{2d} and Ar^{2e}, which independently represent phenyl or a 5- or 6-membered heteroaromatic ring, can further be optionally substituted with at least one substituent, which at least one substituent is as defined in formula (I) for further optional substituents on Ar².

Where Ar² is substituted by CO₂R⁶, MC₁₋C₆alkylCO₂R⁷ or NR²⁷R²⁸ wherein R²⁷ and R²⁸ together with the nitrogen atom to which they are attached form a 3- to 8-membered saturated heterocyclic ring, which heterocyclic ring is substituted by CO₂R⁶ or MC₁C₆alkylCO₂R⁷, a compound of the invention wherein R⁶ or R⁷ represent a C₁-C₆ alkyl group may be converted into a compound of the invention wherein R⁶ or R⁷ represents hydrogen by reaction with a base such as sodium hydroxide or lithium hydroxide in a solvent such as water or methanol, at a temperature in the range 0-150° C., or by reaction with an acid such as hydrochloric acid, hydrobromic acid or trifluoroacetic acid in a solvent such as water, 1,4-dioxane, teteahydrofuran, acetic acid or dichloromethane, at a temperature in the range 0-150° C.

Where Ar² is substituted by carboxyl, a compound of the invention may be converted into a compound of the invention where Ar² is substituted by

C₁₋C₆alkylsulphonylaminocarbonyl by reaction with, for example, methanesulfonamide in the presence of reagents such as 1-(3-dimethylaminopropyl)-3-ethylcarbodiimide hydrochloride and 4-dimethylaminopyridine).

Compounds of formula (VI)-(IX), wherein Y represents an organoboron group such as B(OH)₂ or B(OⁱPr)₂, may be prepared by reacting compounds of formula (VI)-(IX), wherein Y represents a displaceable group such as bromo or iodo, with suitable organometallic reagents, for example methyllithium and tert-butyllithium, in the presence of a trialkylborate, e.g. triisopropylborate, in the presence of a suitable solvent such as tetrahydrofuran, and at a temperature in the range −100° C.-30° C., and optionally followed by hydrolysis of the boronate ester by reaction with an acid such as ammonium chloride in a solvent such as water or tetrahydrofuran, at a temperature in the range 0-150° C.

Alternatively, compounds of formula (VI)-(IX), wherein Y represents an organoboron group such as B(OH)₂ or a boronic acid pinacol cyclic ester may be prepared by reacting compounds of formula (VI)-(IX), wherein Y represents a displaceable group such as a halogeno or sulphonyloxy group, for example a chloro, bromo, iodo, trifluoromethanesulphonyloxy, methanesulphonyloxy or paratoluenesulphonyloxy group, with a suitable diboron reagent, e.g. bis(pinacolato)diboron, in the presence of a catalyst, for example palladium acetate or [1,1′-bis(diphenylphosphino)ferrocene]palladium(II) chloride, in the presence of a base such as potassium acetate or tripotassium phosphate, in the presence of a suitable solvent, e.g. dimethylsulphoxide, 1,4-dioxane or tetrahydrofuran, and at a temperature in the range 25-250° C., and optionally followed by hydrolysis of the boronate ester by reaction with an acid such as ammonium chloride in a solvent such as water or tetrahydrofuran, at a temperature in the range 0-150° C.

Compounds of formula (VI)-(IX), wherein Y represents a leaving group such as a halogeno or sulphonyloxy group, may conveniently be prepared by reacting a compound of general formula

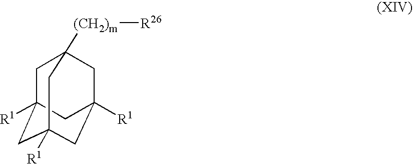

with a compound of general formula

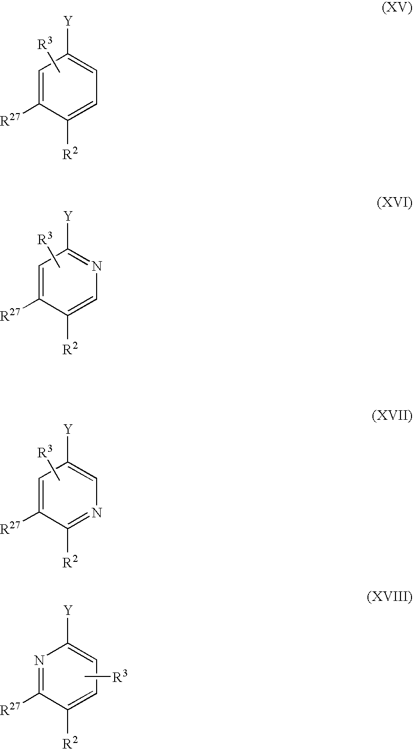

wherein one of R²⁶ and R²⁷ represents NH₂ and the other of R²⁶ and R²⁷ represents CO₂H, COBr or COCl, R¹ and m are as defined in formula (I), Y represents a leaving group such as a halogeno or sulphonyloxy group as defined in formulae (VI)-(IX), and R² and R³ are as defined in formula (I), optionally in the presence of suitable coupling reagents such as 1,1′-carbonyldiimidazole or dicyclohexylcarbodiimide and 1-hydroxybenzotriazole. Compounds of formula (XX) may be prepared by reacting a compound of formula

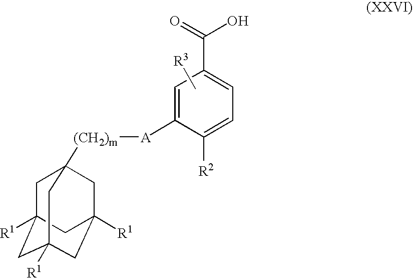

with L-serine methyl ester, in the presence of suitable coupling reagents such as 1,1′-carbonyldiimidazole or 1-(3-dimethylaminopropyl)-3-ethylcarbodiimide hydrochloride and 1-hydroxybenzotriazole.

Compounds of formula (XXVI) may be prepared by reacting a compound of formula

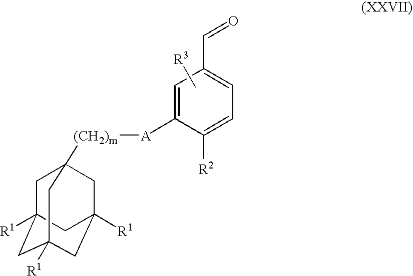

with a suitable oxidant such as potassium peroxomonosulfate or sodium chlorite.

Compounds of formula (XXIX)-(XXXII) where R³⁰ is a carboxyl group may be prepared by reacting a compound of general formula

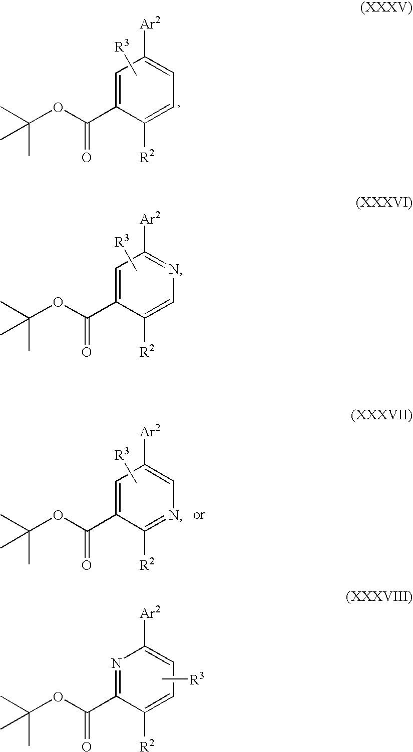

wherein Ar², R², and R³ are as defined in formula (I), with an acid such as hydrochloric acid, hydrobromic acid or trifluoroacetic acid in a solvent such as water, 1,4-dioxane, tetrahydrofuran, acetic acid or dichloromethane, at a temperature in the range 0-150° C.

Compounds of formula (XXXV)-(XXXVIII) may be prepared by reacting a compound of formula

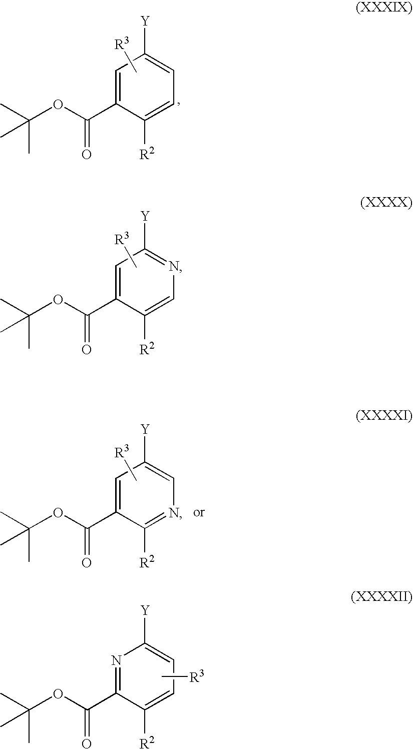

with a compound of formula (X) as defined in (a) above wherein Y is as defined in formula (VI)-(IX), and R² and R³ are as defined in formula (I), in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(H) chloride, in the presence of a suitable solvent such as tetrahydrofuran, 1,4-dioxane, 1,2-dimethoxyethane, benzene, toluene, xylene, methanol, ethanol or water. The reaction may be conducted in the presence of a suitable base such as sodium carbonate or potassium carbonate, pyridine, 4-dimethylaminopyridine, triethylamine or morpholine, and at a temperature in the range 10 to 250° C., e.g. in the range 60 to 120° C.

Compounds of formula (XXXIX)-(XXXXII), wherein Y represents an organoboron group such as B(OH)₂ or B(OⁱPr)₂, may be prepared by reacting compounds of formula (XXXIX)-(XXXXII), wherein Y represents a displaceable group such as bromo or iodo, with suitable organometallic reagents, for example methyllithium and tert-butyllithium, in the presence of a trialkylborate, e.g. triisopropylborate, in the presence of a suitable solvent such as tetrahydrofuran, and at a temperature in the range −100° C. to 30° C., and optionally followed by hydrolysis of the boronate ester by reaction with an acid such as ammonium chloride in a solvent such as water or tetrahydrofuran, at a temperature in the range 0-150° C.

Alternatively, compounds of formula (XXXIX)-(XXXXII), wherein Y represents an organoboron group such as B(OH)₂ or a boronic acid pinacol cyclic ester may be prepared by reacting compounds of formula (XXXIX)-(XXXXII), wherein Y represents a displaceable group such as a halogeno or sulphonyloxy group, for example a chloro, bromo, iodo, trifluoromethanesulphonyloxy, methanesulphonyloxy or paratoluenesulphonyloxy group, with a suitable diboron reagent, e.g. bis(pinacolato)diboron, in the presence of a catalyst, for example palladium acetate or [1,1′-bis(diphenylphosphino)ferrocene]palladium(II) chloride, in the presence of abase such as potassium acetate or tripotassium phosphate, in the presence of a suitable solvent, e.g. dimethylsulphoxide, 1,4-dioxane or telxahychofuran, and at a temperature in the range 25-250° C., and optionally followed by hydrolysis of the boronate ester by reaction with an acid such as ammonium chloride in a solvent such as water or tetrahydrofuran, at a temperature in the range 0-150° C.

Compounds of formula (XXXIX)-(XXXXII), wherein Y represents a leaving group such as a halogeno or sulphonyloxy group, may conveniently be prepared by reacting a compound of formula

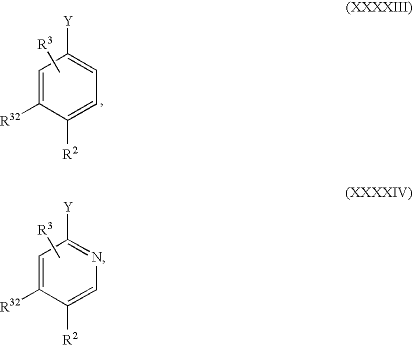

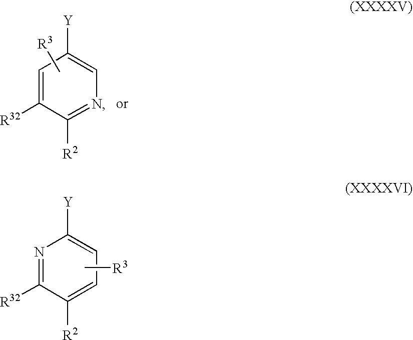

wherein R³² represents CO₂H, COBr or COCl, Y is a leaving group as defined in formula (VI)-(IX),and R² and R³ are as defined in formula (I), with tert-butanol or potassium tert-butoxide, optionally in the presence of suitable reagents such as dicyclohexylcarbodiimide and 4-dimethylaminopyridine.

Compounds of formula (X), (XI), (XII), (XIII), (XIV), (XV), (XVI), (XVII), (XVIII), (XXI), (XXII), (XXIII), (XXIV), (XXV), (XXVII), (XXVIII), (XXXIII), (XXXIV), (XXXXIII), (XXXXIV), (XXXXV) and (XXXXVI) are either commercially available, are known in the literature or may be prepared easily using known techniques.

In processes (a), (b), (c), (d), (e), (f), (g), (i), (j), (k), (l), (m), (o) and (p), the coupling reaction is conveniently carried out in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C.

In processes (c), (f) and (l), the tetrazole formation reaction is carried out in the presence of a suitable source of azide, for example sodium azide, ammonium azide, azidotrimethylsilane or azidotributyltin, optionally in the presence of a suitable catalyst, for example dibutyltin oxide, in the presence of a suitable solvent such as toluene, N,N-dimethylformamide or 1-methyl-2-pyrrolidinone, and at a temperature in the range 10 to 250° C., preferably in the range 50 to 120° C.

In process (d), the displacement reaction may be carried out in the presence of a suitable base, for example potassium tert-butoxide, sodium hydride, potassium carbonate or caesium carbonate, optionally in the presence of a suitable catalyst, for example a palladium catalyst such as tetrakis(triphenylphosphine)palladium(0), palladium(II) chloride, palladium(II) bromide, palladium(II) acetate, dichlorobis(triphenylphosphine)palladium(II) or tris(dibenzylideneacetone)palladium(0), or a copper catalyst such as copper(I) iodide, optionally in the presence of a suitable ligand, for example 1,1′-bis(diphenylphosphino)ferrocene, 9,9-dimethyl-4,5-bis(diphenylphosphino)xanthene or 2-dicyclohexylphosphino-2′-(N,N-dimethylamino)biphenyl, in the presence of a suitable solvent, for example 1-methyl-2-pyrrolidinone, 1,4-dioxane, 1,2-dimethoxyethane, tetrahydrofuran or acetonitrile, and at a temperature in the range 10 to 250° C., preferably in the range 60 to 150° C.

In processes (e) and (f), the displacement reaction may be carried out in the presence of a suitable source of cyanide, for example sodium cyanide, potassium cyanide, copper cyanide or zinc cyanide, optionally in the presence of a suitable catalyst, for example a palladium catalyst such as tetrakis(triphenylphosphine)palladium(0) or palladium(II) acetate, in the presence of a suitable solvent, for example N,N-dimethylformamide, 1-methyl-2-pyrrolidinone or dimethylsulfoxide, and at a temperature in the range 10-250° C., preferably in the range 60 to 150° C.

In process (g), the carbonylation reaction may be carried out in the presence of an alcohol such as butanol, propanol, ethanol or methanol, in the presence of a catalyst such as tetrakis(triphenylphosphine)palladium(0), palladium(II) chloride, palladium(H) bromide, palladium(H) acetate, dichlorobis(triphenylphosphine)palladium (II) or [1,1′-bis(diphenylphosphino)ferrocene]palladium(II) chloride, optionally in the presence of a ligand such as triphenylphosphine or 1,3-bis(diphenylphosphino)propane, in the presence of a suitable base, for example triethylamine, optionally in the presence of a co-solvent, for example 1-methyl-2-pyrrolidinone or N,N-dimethylformamide, and at a temperature in the range 10-150° C., and under a pressure of carbon monoxide in the range of 100 kPa to 1000 kPa (1 to 10 bar).

In process (h), the cyclodehydration reaction may be carried out in the presence of a cyclodehydrating reagent such as diethylaminosulfur trifluoride or Burgess reagent ((methoxycarbonylsulfamoyl)triethylammonium hydroxide, inner salt), in the presence of a suitable solvent, for example dichloromethane, and at a temperature in the range −78-30° C. The oxidation reaction may be carried out in the presence of oxidising reagents such as bromotrichloromethane and 1,8-diazabicyclo[5.4.0]undec-7-ene, or manganese dioxide, in the presence of a suitable solvent such as dichloromethane, at a temperature in the range 0-150° C.

In processes (i) and (j), the displacement reaction may optionally be carried out in the presence of a suitable catalyst such as palladium(II) acetate, optionally in the presence of a suitable ligand such as 9,9-dimethyl-4,5-bis(diphenylphosphino)xanthene or 2-dicyclohexylphosphino-2′-(N,N-dimethylamino)biphenyl, in the presence of a suitable base, for example potassium tert-butoxide, sodium tert-butoxide, triethylamine or potassium carbonate, in the presence of a suitable solvent, for example tetrahydrofuran, acetonitrile, N-methylpyrroldinone, toluene or acetone, and at a temperature in the range 0-150° C.

In processes (k) and (l), the displacement reaction may be carried out in the presence of a suitable base, for example potassium tert-butoxide, triethylamine or potassium carbonate, in the presence of a suitable solvent, for example tetrahydrofuran, acetonitrile, N-methylpyrroldinone or acetone, and at a temperature in the range 0-150° C.

In process (m), the oxidation reaction may be carried out in the presence of an oxidising agent such as potassium peroxymonosulfate or sodium chlorite in a solvent such as N,N-dimethylformamide at a temperature in the range 0-100° C.

In process (n), the amide coupling reaction may be carried out in the presence of a suitable coupling reagent, such as 1,1′-carbonyldiimidazole or dicyclohexylcarbodiimide and 1-hydroxybenzotriazole, in the presence of a base such as triethylamine, N-methylmorpholine, diisopropylethylamine or potassium carbonate, in a solvent such as dichloromethane, N-methylpyrrolidinone, N-N-dimethylformamide or tetrahydrofuran, and at a temperature in the range 0-150° C.

In processes (o) and (p), the displacement reaction may optionally be carried out in the presence of a suitable base, such as triethylamine, in a solvent such as acetonitrile or pyridine, and at a temperature in the range 0-150° C.

It will be appreciated by those skilled in the art that in the processes of the present invention certain functional groups such as hydroxyl, carboxyl or amino groups in the starting reagents or intermediate compounds may need to be protected by protecting groups. Thus, the preparation of the compounds of formula (I) may involve at a certain stage the removal of one or more protecting groups.

The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994).

The compounds of formula (I) above may be converted to a pharmaceutically acceptable salt or solvate thereof. Where the compound is sufficiently acidic, suitable salts include base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Where the compound is sufficently basic, suitable salts include acid addition salts such as a hydrochloride, hydrobromide, phosphate, acetate, fumarate, maleate, tartrate, citrate, oxalate, methanesulphonate or p-toluenesulphonate salt. There may be more than one cation or anion depending on the number of charged functions and the valency of the cations or anions. Other pharmaceutically acceptable salts, as well as prodrugs such as pharmaceutically acceptable esters and pharmaceutically acceptable amides may be prepared using conventional methods. It will be understood that certain compounds of the present invention may exist in solvated, for example hydrated, as well as unsolvated forms. It is to be understood that the present invention encompasses all such solvated forms.

A compound of the invention, or a pharmaceutically acceptable salt or solvate thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis , inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention provides a compound of formula (I), or a pharmaceutically acceptable salt or solvate thereof, as hereinbefore defined for use in therapy.

In another aspect, the invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt or solvate thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or solvate thereof, as hereinbefore defined to a patient.

The compounds of the present invention are especially advantageous as pharmaceuticals for use in the treatment of inflammatory disorders such as rheumatoid arthritis, osteoarthritis, asthma and chronic obstructive pulmonary disease (COPD). Accordingly, the present invention provides for the use of a compound of formula (IA), or a pharmaceutically acceptable salt or solvate thereof, in the manufacture of a medicament for use in the treatment of an inflammatory disorder

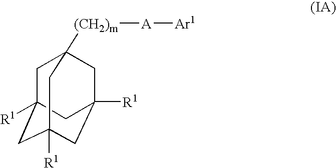

wherein m represents 1,2 or 3;

each R¹ independently represents a hydrogen atom or a halogen;

A represents C(O)NH or NHC(O);

Ar¹ represents a group

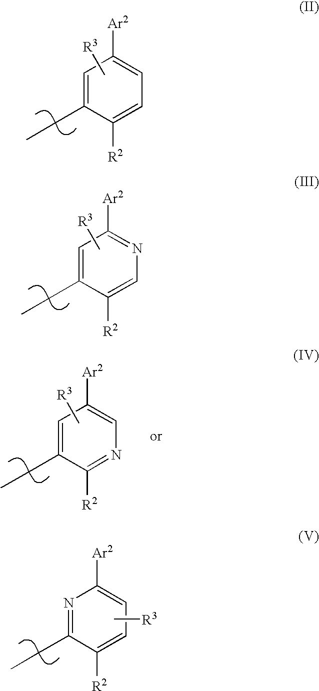

one of R² and R³ represents halogen, nitro, NR⁴R⁵, hydroxyl, or a group selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;

R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;

Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁₋₆ alkylCO₂R⁷, C₁₋₆ alkylsulphonylaminocarbonyl, NHR⁸, R⁹, XR¹⁰, C(O)NHOH and NR²⁸R²⁹;

and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by a halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;

R⁶ and R⁷ each independently represent a hydrogen atom or a C₁₋C₆ alkyl group;

R⁸ represents CN, C₁₋C₆ alkylsulphonyl, C₁₋C₆ alkylcarbonyl, C₁₋C₆ alkoxycarbonyl, C₁₋C₆ alkylaminosulphonyl, or (di)-C₁₋C₆ alkylaminosulphonyl;

R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;

M represents a bond, oxygen, S(O)_{q} or NR²³;

X represents oxygen, S(O)ₛ, NR²⁴, C₁₋C₆ alkylene, O(CH₂)₁₋₆, NR²⁵(CH₂)₁₋₆, or S(O)ₜ(CH₂)₁₋₆;

p, q, s and t each independently represent 0,1 or 2;

R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁₋C₆ alkylCO₂R⁷, C₁₋C₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and

R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl.

The present invention further provides for the use of compound of formula (IA), or a pharmaceutically acceptable salt or solvate thereof, in the manufacture of a medicament for use in the treatment of atherosclerosis.

The present invention further provides a method of treating an inflammatory disorder (e.g. rheumatoid arthritis, osteoarthritis, asthma or chronic obstructive pulmonary disease) or atherosclerosis, which comprises administering a therapeutically effective amount of a compound of formula (IA), or a pharmaceutically acceptable salt or solvate thereof, as hereinbefore defined to a patient.

For all the above-mentioned therapeutic uses the dosage administered will, of course, vary with the compound employed, the mode of administration, the treatment desired and the disorder indicated. The daily dosage of the compound of formula (I)/salt/solvate (“active ingredient”) may be in the range from 0.001 mg/kg to 30 mg/kg.

The compounds of formula (I) and pharmaceutically acceptable salts or solvates thereof may be used on their own but will generally be administered in the form of a pharmaceutical composition in which the formula (I) compound/salt/solvate (“active ingredient”) is in association with a pharmaceutically acceptable adjuvant, diluent or carrier. Depending on the mode of administration, the pharmaceutical composition will preferably comprise from 0.05 to 99% w (per cent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Thus, the present invention also provides a pharmaceutical composition comprising a compound of formula (I), or a pharmaceutically acceptable salt or solvate thereof, as hereinbefore defined in association with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The invention further relates to combination therapies wherein a compound of the invention, or a pharmaceutically acceptable salt or solvate thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt or solvate thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt or solvate thereof, with a monoclonal antibody targeting B-Lymphocytes (such as CD20 (rituximab), MRA-aIL16R and T-Lymphocytes, CTLA4-Ig, HuMax II-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt or solvate thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt or solvate thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BILL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt or solvate thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt or solvate thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt or solvate thereof, and a proton pump inhibitor (such as omeprazole) or a gastroprotective histamine type 2 receptor antagonist.

The present invention further relates to the combination of a compound of the invention, or a pharmaceutically acceptable salt or solvate thereof, and an antagonist of the histamine type 4 receptor.

The present invention still further relates to the combination of a compound of the invention, or a pharmaceutically acceptable salt or solvate thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt or solvate thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt or solvate thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt or solvate thereof, and a chromone, such as sodium cromoglycate or nedocromil sodium.

The present invention still further relates to the combination of a compound of the invention, or a pharmaceutically acceptable salt or solvate thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt or solvate thereof, with an agent that modulates a nuclear hormone receptor such as PPARs.

The present invention still further relates to the combination of a compound of the invention, or a pharmaceutically acceptable salt or solvate thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt or solvate thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt or solvate thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt or solvate thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt or solvate thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt or solvate thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt or solvate thereof, and an agent for the treatment of acute or chronic pain, such as a-centrally or peripherally-acting analgesic (for example an opioid or derivative thereof), carbamazepine, phenytoin, sodium valproate, amitryptiline or other anti-depressant agent-s, paracetamol, or a non-steroidal anti-inflammatory agent.

The present invention further relates to the combination of a compound of the invention, or a pharmaceutically acceptable salt or solvate thereof, together with a parenterally or topically-applied (including inhaled) local anaesthetic agent such as lignocaine or a derivative thereof.

A compound of the present invention, or a pharmaceutically acceptable salt or solvate thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt or solvate thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2. -receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; or (xxvii) inhibitor of transcription factor activation such as NFkB, API, or STATS.

A compound of the invention, or a pharmaceutically acceptable salt or solvate thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples. In the examples the NMR spectra were measured on a Varian Unity spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% aqueous ammonium acetate: acetonitrile as the eluant. Microwave reactions were performed in a CEM Discover single mode microwave. In the following examples all compounds were named using the Chemical Abstracts Service Index Name function within the ACD/Name software package.

### EXAMPLE 1

### 4′-Chloro-3′-[[(tricyclo[3.3.1.1^{3,7}]dec-1-ylmethyl)amino]carbonyl]-[1,1′-biphenyl]-4-carboxylic acid

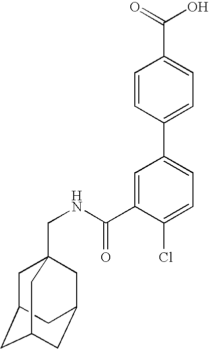

A mixture of 2-chloro-5-iodo-N-(tricyclo[3.3.1.1^{3,7}]dec-1-ylmethyl)-benzamide (Prepared as described in WO200144170) (200 mg), 4-carboxyphenylboronic acid (83 mg), potassium carbonate (140 mg) and dichlorobis(triphenylphosphine)palladium (II) (35 mg) in 1,4-dioxane (2 mL)/water (1 mL) was heated at 80° C. under a nitrogen atmosphere for 3 hours. The products were filtered through diatomaceous earth, washing with methanol (2×10 mL). The solvent was removed in vacuo and the residue was purified by chromatography (SiO₂, dichloromethane methanol 97:3 as eluant) and then by recrystallisation (acetonitrile) to yield the title compound as a colourless solid (150 mg).

MS: APCI(−ve) 422/424 (M−H⁺).

m.p. 267-270° C. dec.

¹H NMR (400 MHz, d₆-DMSO) δ 13.03 (1H, s), 8.42 (1H, t), 8.04 (2H, d), 7.84 (2H, d), 7.80 (1H, dd), 7.72 (1H, d), 7.61 (1H, d), 2.98 (2H, d), 1.95 (3H, s), 1.74-1.57 (6H, m), 1.55 (6H, s).

### EXAMPLE 2

### 4′-Chloro-3′-[[(tricyclo[3.3.1.1^{3,7}]dec-1-ylmethyl)amino]carbonyl]-[1,1′-biphenyl]-3-carboxylic acid

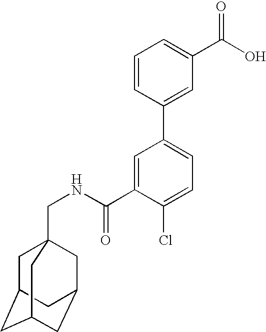

a) [4-Chloro-3-[[(tricyclo[3.3.1.1^{3,7}]dec-1-ylmethyl)amino]carbonyl]phenyl]-boronic acid

Methyllithium (1.6M in diethyl ether, 9.5 mL) was added to a stirred solution of 5-bromo-2-chloro-N-(tricyclo[3.3.1.1^{3,7}]dec-1-ylmethyl)-benzamide (Prepared as described in WO200144170) (4.8 g) in tetrahydrofuran (120 mL) at −78° C. under an atmosphere of nitrogen. After 10 minutes, triisopropyl borate (15 mL) was added, followed by tert-butyllithium (1.7M in pentane, 17 mL). After stirring at −78° C. for 2 hours, saturated aqueous ammonium chloride (125 mL) was cautiously added and the mixture was allowed to warm to room temperature over 16 hours. Ethyl acetate (500 mL) was added and the organic fraction was washed with water (3×250 mL) before being dried (MgSO₄), filtered and concentrated in vacuo to yield the sub-title compound as a colourless solid (4.2 g).

MS: APCI(+ve) 348/350 (M+H⁺).

b) 4′-Chloro-3′-[[(tricyclo[3.3.1.1^{3,7}]dec-1-ylmethyl)amino]carbonyl]-[1,1′-biphenyl]-3-carboxylic acid

Prepared according to the method of Example 1 using [4-chloro-3-[[(tricyclo[3.3.1.1^{3,7}]dec-1-ylmethyl)amino]carbonyl]phenyl]-boronic acid (Example 2 (a)) (300 mg), 3-iodobenzoic acid (210 mg), potassium carbonate (240 mg) and dichlorobis(triphenylphosphine)palladium (II) (60 mg) to yield the title compound as a solid (40 mg).

MS: APCI(−ve) 422/424 (M−H⁺).

m.p. 255-257° C.

¹H NMR (400 MHz, d₆-DMSO) δ 13.15 (1H, s), 8.42 (1H, t), 8.2 (1H, t), 7.99-7.95 (2H, m), 7.77 (1H, dd), 7.68 (1H, d), 7.63 (1H, t), 7.60 (1H, d), 2.97 (2H, d), 1.95 (3H, s), 1.69-1.59 (6H, m), 1.54 (6H, s).

### EXAMPLE 3

### 4′-Chloro-3′-[[(tricyclo[3.3.1.1^{3,7}]dec-1-ylmethyl)amino]carbonyl]-[1,1′-biphenyl]-2-carboxylic acid

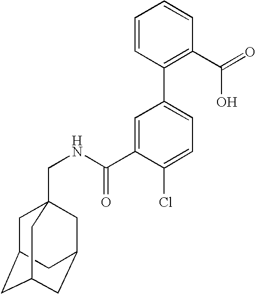

a) 4′-Chloro-3′-[[(tricyclo[3.3.1.1^{3,7}]dec-1-ylmethyl)amino]carbonyl]-[1,1′-biphenyl]-2-carboxylic acid, ethyl ester

Prepared according to the method of Example 1 using [4-chloro-3-[[(tricyclo[3.3.1.1^{3,7}]dec-1-ylmethyl)amino]carbonyl]phenyl]-boronic acid (Example 2 (a)) (500 mg), ethyl 2-iodobenzoate (600 mg), potassium carbonate (600 mg) and dichlorobis(triphenylphosphine)palladium (II) (50 mg) to give the sub-title compound as a solid (350 mg).

MS: APCI(+ve) 452/454 (M+H⁺).

b) 4′-Chloro-3′-[[(tricyclo[3.3.1.1^{3,7}]dec-1-ylmethyl)amino]carbonyl]-[1,1′-biphenyl]-2-carboxylic acid

A mixture of 4′-chloro-3′-[[(tricyclo[3.3.1.1^{3,7}]dec-1-ylmethyl)amino]carbonyl]-[1,1′-biphenyl]-2-carboxylic acid, ethyl ester (Example 3 (a)) (350 mg), methanol (2 mL) and aqueous sodium hydroxide (2.5M, 2.0 mL) was heated in a microwave at 70° C. for 2 hours. The volatile components were removed in vacuo and the residue partitioned between aqueous sodium hydroxide (2M, 20 mL) and dichloromethane (20 mL). The aqueous fraction was acidifed to pH 3 with hydrochloric acid (2M) and extracted with ethyl acetate (2×20 mL). The combined organics were dried (MgSO₄), filtered and concentrated. Recrystallisation (acetonitrile) gave the title compound as a colourless solid (15 mg).

MS: APCI(−ve) 422/424 (M−H⁺).

m.p. 228-230° C.

¹H NMR (400 MHz, d₆-DMSO) δ 12.94 (1H, s), 8.38 (1H, t), 7.78 (1H, dd), 7.61 (1H, ddd), 7.51 (1H, d), 7.50 (1H, ddd), 7.41 (1H, dd), 7.38 (1H, dd), 7.34 (1H, d), 2.94 (2H, d), 1.94 (3H, s), 1.70-1.56 (6H, m), 1.53-1.51 (6H, m).

### EXAMPLE 4

### 2-Chloro-5-[6-(cyanoamino)pyrazinyl]-N-(tricyclo[3.3.1.1^{3,7}]dec-1-ylmethyl)-benzamide

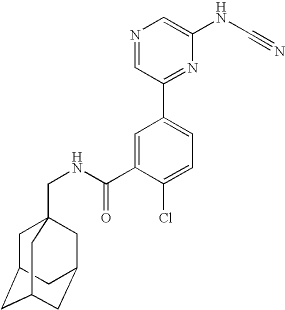

A mixture of [4-chloro-3-[[(tricyclo[3.3.1.1^{3,7}]dec-1-ylmethyl)amino]carbonyl]phenyl]-boronic acid (Example 2 (a)) (500 mg), 2,6-dichloropyrazine (900 mg), potassium carbonate (350 mg) and dichlorobis(triphenylphosphine)palladium (II) (90 mg) in 1,4-dioxane (5 mL)/water (2 mL) was heated at 80° C. under a nitrogen atmosphere for 1 hour. The products were filtered through diatomaceous earth, washing with methanol (2×10 mL). The solvent was removed in vacuo and the residue was purified (SiO₂, dichloromethane:methanol 99:1 as eluant) to give a solid which was taken up in acetonitrile (1.5 mL) and sodium hydrogen cyanamide (270 mg) was added. The mixture was heated in a microwave at 100° C. for 3 hours and then purified (SiO₂, dichloromethane:methanol 95:5 as eluant). Further purification (RP-HPLC, acetonitrile:aqueous trifluoroacetic acid, Symmetry) gave the title compound as a colourless solid (26 mg).

MS: APCI(+ve) 422/424 (M+H⁺), 439/441 (M+NH₃+H⁺)

m.p. 215-220° C. dec.

¹H NMR (400 MHz, d₆-DMSO) δ 8.41 (1H, t), 8.08 (1H, s), 8.04 (1H, d), 8.00 (1H, s), 7.62 (1H, s), 7.54 (1H, d), 2.97 (2H, d), 1.95 (3H, s), 1.69-1.61 (6H, m), 1.55 (6H, s).

### EXAMPLE 5

### 2-Chloro-5-[3-(cyanamino)pyrazinyl]-N-(tricyclo[3.3.1.1^{3,7}]dec-1-ylmethyl)-benzamide

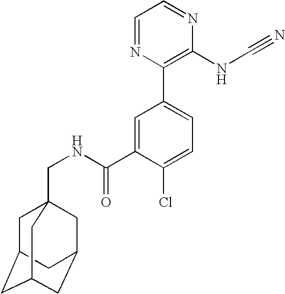

a) 2-Chloro-5-(3-chloropyrazinyl)-N-(tricyclo[3.3.1.1^{3,7}]dec-1-ylmethyl)-benzamide

Prepared according to the method of Example 1, using [4-chloro-3-[[(tricyclo[3.3.1.1^{3,7}]dec-1-ylmethyl)amino]carbonyl]phenyl]-boronic acid (Example 2 (a)) (1.0 g), 2,3-dichloropyrazine (1.8 g), potassium carbonate (700 mg) and dichlorobis(triphenylphosphine)palladium (II) (180 mg). Ethyl acetate (100 mL) and water (50 mL) were added and the organic fraction was washed with hydrochloric acid (2M, 50 mL) and saturated sodium chloride (50 mL) before being dried (MgSO₄) and evaporated. Purification by chromatography (SiO₂, dichloromethane:methanol 99:1 as eluant) gave the sub-title compound as a solid (750 mg).

MS: APCI(+ve) 416/418 (M+H⁺)

b) 2-Chloro-5-[3-(cyanamino)pyrazinyl]-N-(tricyclo[3.3.1.1^{3,7}]dec-1-ylmethyl)-benzamide

A mixture of 2-chloro-5-(3-chloropyrazinyl)-N-(tricyclo[3.3.1.1^{3,7}]dec-1-ylmethyl)-benzamide (Example 5 (a)) (340 mg), sodium hydrogen cyanamide (280 mg), tetrabutylammonium bromide (50 mg) and acetonitrile (2 mL) was heated in a microwave at 100° C. for 3 hours. Purification (SiO₂, dichloromethane:methanol:trifluoroacetic acid 96:3:1 as eluant) gave the title compound as a solid (76 mg).

MS: APCI(+ve) 422/424 (M+H⁺)

m.p. 110-115° C. dec.

¹H NMR (400 MHz, d₆-DMSO, 363 K) δ 8.24-8.14 (1H, m), 8.11-8.02 (2H, m), 7.92-7.83 (2H, m), 7.58 (1H, d), 2.98 (2H, d), 1.95 (3H, br s), 1.73-1.58 (6H, m), 1.55 (6H, s).

### EXAMPLE 6

### 3-[4-Chloro-3-[[(tricyclo[3.3.1.1^{3,7}]dec-1-ylmethyl)amino]carbonyl]phenyl]-2-pyrazinecarboxylic acid, monosodium salt

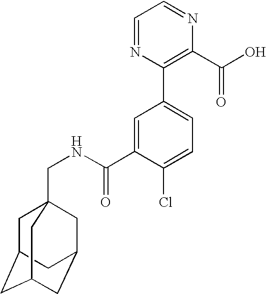

a) 2-Chloro-5-(3-cyanopyrazinyl)-N-(tricyclo[3.3.1.1^{3,7}]dec-1-ylmethyl)-benzamide

Prepared according to the method of Example 1, using [4-chloro-3-[[(tricyclo[3.3.1.1^{3,7}]dec-1-ylmethyl)amino]carbonyl]phenyl]-boronic acid (Example 2 (a)) (0.5 g), 3-chloro-2-pyrazinecarbonitrile (0.6 g), potassium carbonate (350 mg) and dichlorobis(triphenylphosphine)palladium (II) (500 mg). The mixture was extracted with ethyl acetate (3×10 mL) and the combined organic fractions were washed with water (10 mL) and saturated sodium chloride (10 mL) before being dried (MgSO₄) and evaporated. Purification by chromatography (SiO₂, dichloromethane as eluant) gave the sub-title compound as a solid (390 mg).

MS: APCI(+ve) 407/409 (M+H⁺)

b) 3-[4-Chloro-3-[[(tricyclo[3.3.1.1^{3,7}]dec-1-ylmethyl)amino]carbonyl]phenyl]-2-pyrazinecarboxylic acid, monosodium salt

A mixture of 2-chloro-5-(3-cyanopyrazinyl)-N-(tricyclo[3.3.1.1^{3,7}]dec-1-ylmethyl)-benzamide (Example 6 (a)) (200 mg), methanol (1 mL) and aqueous sodium hydroxide (6M, 1.0 mL) was heated in a microwave at 80° C. for 2 hours. Purification by chromatography (SiO₂, dichloromethane:methanol:trifluoroacetic acid 96:4:1 as eluant), redissolution in acetonitrile (2 mL) and formation of the sodium salt by addition of sodium hydroxide (1M, 0.47 mL) and filtration of the resulting precipitate gave the title compound as a solid (90 mg).

MS: APCI(+ve) 426/428 (M+H⁺).

m.p. 194-195° C. dec.

¹H NMR (400 MHz, d₆-DMSO) δ 8.87 (1H, d), 8.71 (1H, d), 8.46 (1H, t), 7.74-7.69 (2H, m), 7.64 (1H, d), 2.95 (2H, d), 1.94 (3H, s), 1.72-1.57 (6H, m), 1.55-1.51 (6H, m).

### EXAMPLE 7

### 3-[5-Chloro-4-[[(tricyclo [3.3.1.1^{3,7}]dec-1-ylmethyl)amino]carbonyl]-2-pyridinyl]-benzoic acid

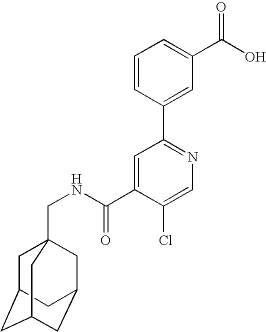

Prepared according to the method of Example 1, using 2,5-dichloro-N-(tricyclo[3.3.1.1^{3,7}]dec-1-ylmethyl)-4-pyridinecarboxamide (Prepared as described in WO200144170) (0.5 g), 3-carboxyphenylboronic acid (0.5 g), potassium carbonate (350 mg) and dichlorobis(triphenylphosphine)palladium (II) (50 mg). Purification (SiO₂, dichloromethane:methanol 97:3 as eluant) and then by recrystallisation (acetonitrile:diethyl ether) gave the title compound as a solid (24 mg).

MS: APCI(+ve) 425/427 (M+H⁺).

m.p. 200-202° C.

¹H NMR (400 MHz, d₆-DMSO) δ 8.80 (1H, s), 8.69 (1H, s), 8.60 (1H, t), 8.36 (1H, d), 8.06 (1H, s), 8.03 (1H, d), 7.65 (1H, dd), 2.99 (2H, d), 1.96 (3H, s), 1.72-1.58 (6H, m), 1.58-1.53 (6H, m).

### EXAMPLE 8

### 2-Chloro-5-[3-[(methylsulfonyl)amino)pyrazinyl]-N-(tricyclo[3.3.1.1^{3,7}]dec-1-ylmethyl)-benzamide

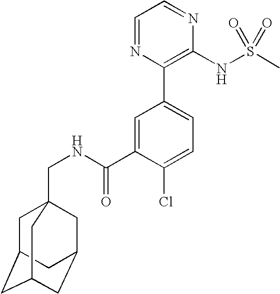

A mixture of 2-chloro-5-(3-chloropyrazinyl)-N-(tricyclo[3.3.1.1^{3,7}]dec-1-ylmethyl)-benzamide (Example 5 (a)) (340 mg), methanesulfonamide (70 mg), potassium tert-butoxide (28 mg) and acetonitrile (1 mL) was heated in a microwave at 140° C. for 4 hours. Purification (Varian NH₂ cartridge using methanol (100 mL) and then 1% trifluoroacetic acid in methanol (100 mL) as eluant, then RP-HPLC, acetonitrile:aqueous ammonium acetate, Symmetry) gave the title compound as a solid (9 mg).

MS: APCI(+ve) 475/477 (M+H⁺)

m.p. 245° C. dec.

¹H NMR (400 MHz, d₆-DMSO) δ 8.52-8.39 (2H, m), 8.36 (1H, t), 7.84-7.76 (2H, m), 7.66-7.60 (1H, m), 3.34 (s, 3H), 2.96 (2H, d), 1.93 (3H, s), 1.70-1.57 (6H, m), 1.52 (6H, s).

### EXAMPLE 9

### 2-Chloro-5-[3(1-H-tetrazol-5-yl)pyrazinyl]-N-(tricyclo[3.3.1.1^{3,7}]dec-1-ylmethyl)-benzamide

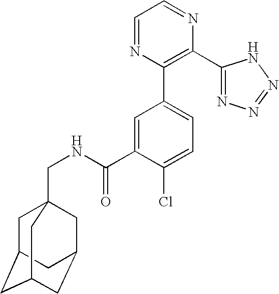

A mixture of 2-chloro-5-(3-cyanopyrazinyl)-N-(tricyclo[3.3.1.1^{3,7}]dec-1-ylmethyl)-benzamide (Example 6 (a)) (180 mg), azidotrimethylsilane (0.15 mL) and dibutyltin oxide (20 mg) in toluene (1 mL) was heated at 95° C. for 16 hours. Further azidotrimethylsilane (0.15 mL) was added and the mixture was heated at 95° C. for 8 hours. Purification (Varian NH₂ cartridge using methanol (100 mL) and then 1% trifluoroacetic acid in methanol (100 mL) as eluant), subsequent recrystallisation (methanol) and washing of the resulting solid with isohexane gave the title compound as a solid (55 mg).

MS: APCI(+ve) 450/452 (M+H⁺)

m.p. 254° C. dec.

¹H NMR (400 MHz, d₆-DMSO) δ 8.96 (1H, d), 8.90 (1H, d), 8.41 (1H, t), 7.57-7.46 (3H, m), 2.93 (2H, d), 1.95 (3H, s), 1.71-1.56 (6H, m), 1.54-1.47 (6H, m).

### EXAMPLE 10

### 2-[4-Chloro-3-[[(tricyclo[3.3.1.1^{3,7}]dec-1-ylmethyl)amino]carbonyl]phenyl]-3-pyridinecarboxylic acid

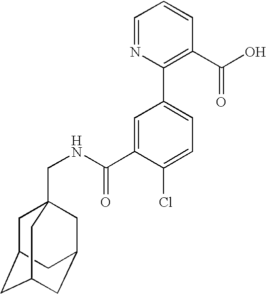

a) 2-[4-Chloro-3-[[(tricyclo[3.3.1.1^{3,7}]dec-1-ylmethyl)amino]carbonyl]phenyl]-3-pyridinecarboxylic acid, methyl ester

A mixture of [4-chloro-3-[[(tricyclo[3.3.1.1^{3,7}]dec-1-ylmethyl)amino]carbonyl]phenyl]-boronic acid (Prepared as described in Example 2 (a)) (200 mg), 2-chloronicotinic acid, methyl ester (118 mg), potassium carbonate (166 mg) and tetrakis(triphenylphosphine)palladium(0) (69 mg) in tetrahydrofuran (2 mL)/water (1 mL) was heated at 65° C. under a nitrogen atmosphere for 1 hour. The products were filtered through diatomaceous earth, washing with methanol (2×10 mL). The solvent was removed in vacuo and the residue was purified (SiO₂, dichloromethane:methanol 99:1 as eluant) to yield the sub-title compound as a solid (200 mg).

MS: APCI(+ve) 439/441 (M+H⁺).

b) 2-[4-Chloro-3-[[(tricyclo[3.3.1.1^{3,7}]dec-1-ylmethyl)amino]carbonyl]phenyl]-3-pyridinecarboxylic acid

Potassium hydroxide (100 mg) in methanol (2 mL) was added to a stirred solution of 2-[4-chloro-3-[[(tricyclo[3.3.1.1^{3,7}]dec-1-ylmethyl)amino]carbonyl]phenyl]-3-pyridinecarboxylic acid, methyl ester (Example 10 (a)) (200 mg) in methanol (1 mL). The reaction was stirred at room temperature for 24 hours then evaporated. The residue was dissolved in water (5 mL) and the solution was acidified to pH 5 with 2 M aqueous hydrochloric acid. The resulting solid was collected by filtration and purified (Varian NH₂ cartridge using methanol (100 mL) and then 5% acetic acid in methanol (100 mL) as eluent) to afford the title compound as a solid (25 mg).

MS: APCI(−ve) 423/425 (M−H⁺).

m.p. 169-172° C.

¹H NMR (400 MHz, d₆-DMSO) δ 8.76 (1H, dd), 8.43 (1H, t), 8.13 (1H, dd), 7.62-7.47 (4H, m), 2.95 (2H, d), 1.94 (3H, s), 1.67 (3H, d), 1.59 (3H, d), 1.52 (6H, s).

### EXAMPLE 11

### 5-[4-Chloro-3-[[(tricyclo[3.3.1.1^{3,7}]dec-1-ylmethyl)amino]carbonyl]phenyl]-3-pyridinecarboxylic acid

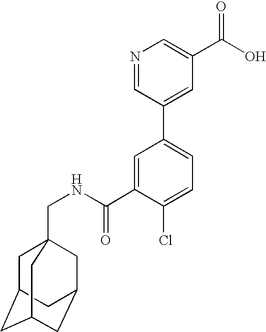

a) 5-[4-Chloro-3-[[(tricyclo[3.3.1.1^{3,7}]dec-1-ylmethyl)amino]carbonyl]phenyl]-3-pyridinecarboxylic acid, methyl ester

Prepared according to the method of Example 10 (a) using [4-chloro-3-[[(tricyclo[3.3.1.1^{3,7}]dec-1-ylmethyl)amino]carbonyl]phenyl]-boronic acid (Prepared as described in Example 2 (a)) (200 mg) and 5-bromo-3-pyridinecarboxylic acid methyl ester (150 mg). Purification (SiO₂, dichloromethane:methanol 99:1 as eluant) afforded the sub-title compound as a solid (100 mg).

MS: APCI(+ve) 439/441 (M+H⁺).

b) 5-[4-Chloro-3-[[(tricyclo[3.3.1.1^{3,7}]dec-1-ylmethyl)amino]carbonyl]phenyl]-3-pyridinecarboxylic acid

Prepared according to the method of Example 10 (b) using 5-[4-chloro-3-[[(tricyclo[3.3.1.1^{3,7}]dec-1-ylmethyl)amino]carbonyl]phenyl]-3-pyridinecarboxylic acid, methyl ester (Example 11 (a)) (100 mg). Purification (Varian NH₂ cartridge using methanol:dichloromethane 1:1 (100 mL) and then acetic acid:methanol:dichloromethane 1:10:10 (100 mL) as eluant) afforded the title compound as a solid (33 mg).

MS: APCI(+ve) 425/427 (M+H⁺).

m.p. 178-182° C.

¹H NMR (400 MHz, d₆-DMSO) δ 9.14 (1H, d), 9.08 (1H, d), 8.50 (1H, t), 8.42 (1H, t), 7.88 (1H, d), 7.85 (1H, d), 7.81 (1H, d), 2.98 (2H, d), 1.95 (3H, s), 1.68 (3H, d), 1.61 (3H, d), 1.55 (6H, s).

### EXAMPLE 12

### 2-[4-Chloro-3-[[(tricyclo[3.3.1.1^{3,7}]dec-1-ylmethyl)amino]carbonyl]phenyl]-4-pyridinecarboxylic acid

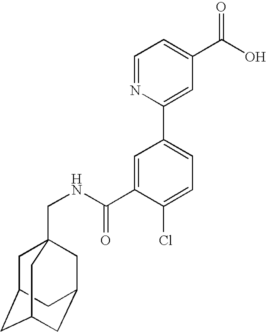

a) 2-[4-Chloro-3-[[(tricyclo[3.3.1.1^{3,7}]dec-1-ylmethyl)amino]carbonyl]phenyl]-4-pyridinecarboxylic acid, 1,1-dimethylethyl ester

Prepared according to the method of Example 10 (a) using [4-chloro-3-[[(tricyclo[3.3.1.1^{3,7}]dec-1-ylmethyl)amino]carbonyl]phenyl]-boronic acid (Prepared as described in Example 2 (a)) (200 mg) and 2-chloro-4-pyridinecarboxylic acid, 1,1-dimethylethyl ester (150 mg). Purification (Si)₂, 99:1 dichloromethane:methanol 99:1 as eluant) afforded the sub-title compound as a solid (150 mg).

MS: APCI(+ve) 481/483 (M+H⁺).

b) 2-[4-Chloro-3-[[(tricyclo[3.3.1.1^{3,7}]dec-1-ylmethyl)amino]carbonyl]phenyl]-4-pyridinecarboxylic acid

Trifluoroacetic acid (1 mL) was added to a stirred solution of 2-[4-chloro-3-[[(tricyclo[3.3.1.1^{3,7}]dec-1-ylmethyl)amino]carbonyl]phenyl]-4-pyridinecarboxylic acid 1,1-dimethylethyl ester (Example 12 (a)) (150 mg) in dichloromethane (3 mL). The reaction was stirred at room temperature overnight then evaporated. Purification (SiO₂, dichloromethane:methanol 99:1 as eluant) afforded the title compound as a solid (17 mg).

MS: APCI(−ve) 423/425 (M−H⁺).

m.p. 284-287° C.

¹H NMR (400 MHz, d₆-DMSO) δ 8.82 (1H, d), 8.45 (1H, t), 8.34 (1H, s), 8.16 (1H, dd), 8.11 (1H, d), 7.80 (1H, d), 7.62 (1H, d), 2.98 (2H, d), 1.95 (3H, s), 1.68 (3H, d), 1.61 (3H, d), 1.55 (6H, s).

### EXAMPLE 13

### 2-[4-Chloro-3-[[(tricyclo[3.3.1.1^{3,7}]dec-1-ylmethyl)amino]carbonyl]phenyl]-6-methyl-3-pyridinecarboxylic acid

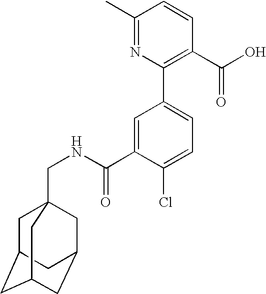

a) 2-Chloro-6-methyl-3-pyridinecarboxylic acid, methyl ester

To a stirred solution of 2-chloro-6-methyl-3-pyridinecarboxylic acid (200 mg) in dichloromethane (2 mL) at 0° C. under nitrogen was added N,N-dimethylformamide (1 drop) and oxalyl chloride (0.3 mL). The reaction mixture was stirred at room temperature for 30 minutes, then evaporated to dryness and redissolved in dichloromethane (2 mL). Methanol (2 mL) was added dropwise and the mixture was stirred at room temperature for 10 minutes before being evaporated to give the sub-title compound as a solid (210 mg).

MS: APCI(+ve) 186/188 (M+H⁺).

¹H NMR (400 MHz, d₆-DMSO) δ 8.16 (1H, d), 7.42 (1H, d), 3.87 (3H, s), 2.52 (3H, s).

b) 2-[4-Chloro-3-[[(tricyclo[3.3.1.1^{3,7}]dec-1-ylmethyl)amino]carbonyl]phenyl]-6-methyl-3-pyridinecarboxylic acid, methyl ester

Prepared according to the method of Example 10 (a) using [4-chloro-3-[[(tricyclo[3.3.1.1^{3,7}]dec-1-ylmethyl)amino]carbonyl]phenyl]-boronic acid (Prepared as described in Example 2 (a)) (200 mg) and 2-chloro-6-methyl-3-pyridinecarboxylic acid methyl ester (Example 13 (a)) (133 mg). Purification (SiO₂, dichloromethane:methanol 99:1 as eluant) afforded the sub-title compound as a solid (130 mg).

MS: APCI(+ve) 453/455 (M+H⁺).

c) 2-[4-Chloro-3-[[(tricyclo[3.3.1.1^{3,7}]dec-1-ylmethyl)amino]carbonyl]phenyl]-6-methyl-3-pyridinecarboxylic acid

Prepared according to the method of Example 10 (b) using 2-[4-chloro-3-[[(tricyclo[3.3.1.1^{3,7}]dec-1-ylmethyl)amino]carbonyl]phenyl]-6-methyl-3-pyridinecarboxylic acid, methyl ester (Example 13 (b)) (130 mg). Purification (Varian NH₂ cartridge using methanol:dichloromethane 1:1 (100 mL) and then acetic acid:methanol:dichloromethane 1:10:10 (100 mL) as eluant) afforded the title compound as a solid (27 mg).

MS: APCI(−ve) 437/439 (M−H⁺).

m.p. 209-211° C.

¹H NMR (400 MHz, CD₃OD) δ 7.98 (1H, d), 7.66-7.61 (2H, m), 7.51 (1H, d), 7.31 (1H, d), 3.07 (2H, s), 2.59 (3H, s), 1.98 (3H, s), 1.77 (3H, d), 1.70 (3H, d), 1.63 (6H, s).

### EXAMPLE 14

### (2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid

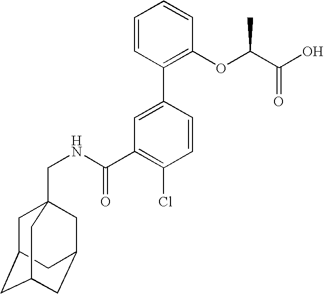

a) 4-Chloro-2′-hydroxy-N-(tricyclo[3.3.1.1^{3,7}]dec-1-ylmethyl)-[1,1′-biphenyl]-3-carboxamide

A mixture of 2-chloro-5-iodo-N-(tricyclo[3.3.1.1^{3,7}]dec-1-ylmethyl)-benzamide (Prepared as described in WO200144170) (1 g), (2-hydroxyphenyl)-boronic acid (450 mg), potassium carbonate (700 mg) and dichlorobis(triphenylphosphine)palladium(II) (175 mg) in tetrahydrofuran (10 mL)/water (10 mL) was heated at 50° C. under a nitrogen atmosphere for 1 hour. The mixture was concentrated and then extracted with dichloromethane (3×50 mL). The combined organic extracts were filtered through diatomaceous earth and evaporated. Purification (SiO₂, dichloromethane methanol 99:1 as eluant) afforded the sub-title compound as a solid (900 mg).

MS: APCI(+ve) 396/398 (M+H⁺).

b) (2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid, methyl ester

Methyl (R)-2-chloropropionate (311 mg) was added to a stirred solution of 4-chloro-2′-hydroxy-N-(tricyclo[3.3.1.1^{3,7}]dec-1-ylmethyl)-[1,1′-biphenyl]-3-carboxamide (Example 14 (a)) (250 mg) and potassium carbonate (174 mg) in acetone (4 mL). The reaction mixture was heated to 55° C. under a nitrogen atmosphere for 10 hours, was then allowed to cool and was concentrated. The residue was partitioned between water (20 mL) and dichloromethane (20 mL). The layers were separated and the aqueous was extracted with dichloromethane (2×20 mL). The combined organics were dried, filtered and evaporated. Purification (SiO₂, dichloromethane as eluant) afforded the sub-title compound as a solid (110 mg).

MS: APCI(+ve) 482/484 (M+H⁺).

¹H NMR (400 MHz, d₆-DMSO) δ 8.39 (1H, t), 7.67-7.60 (2H, m), 7.52 (1H, d), 7.39-7.29 (2H, m), 7.07 (1H, t), 6.96 (1H, d), 5.10 (1H, q), 3.67 (3H, s), 3.00-2.89 (2H, m), 1.93 (3H, s), 1.67 (3H, d), 1.59 (3H, d), 1.53 (6H, s), 1.45 (3H, d).

c) (2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid

Prepared according to the method of Example 10 (b) using (2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid, methyl ester (Example 14 (b)) (110 mg). The residue was dissolved in deionised water (5 mL) and the solution was acidified to pH 5 with 2 M aqueous hydrochloric acid. The resulting solid was collected by filtration and washed with water to give the title compound as a solid (75 mg).

MS: APCI(+ve) 468/470 (M+H⁺).

m.p. 107-110° C.

¹H NMR (400 MHz, d₆-DMSO) δ 8.39 (1H, t), 7-70-7.63 (2H, m), 7.51 (1H, d), 7.38-7.29 (2H, m), 7.05 (1H, t), 6.93 (1H, d), 4.93 (1H, q), 2.94 (2H, d), 1.93 (3H, s), 1.66 (3H, d), 1.59 (3H, d), 1.52 (6H, s), 1.45 (3H, d).

### EXAMPLE 15

### [[4′-Chloro-3′-[[(tricyclo[3.3.1.1^{3,7}]dec-1-ylmethyl)amino]carbonyl][1,1′-biphenyl]-2-yl]oxy]-acetic acid

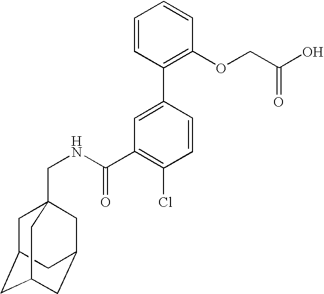

a) [[4′-Chloro-3′-[[(tricyclo[3.3.1.1^{3,7}]dec-1-ylmethyl)amino]carbonyl][1,1′-biphenyl]-2-yl]oxy]-acetic acid, ethyl ester

Ethyl chloroacetate (300 mg) was added to a stirred solution of 4-chloro-2′-hydroxy-N-(tricyclo[3.3.1.1^{3,7}]dec-1-ylmethyl)-[1,1′-biphenyl]-3-carboxamide (Example 14 (a)) (250 mg) and potassium carbonate (174 mg) in acetone (4 mL). The reaction mixture was heated to 55° C. under a nitrogen atmosphere for 1 hour, then allowed to cool and concentrated. The residue was partitioned between deionised water (20 mL) and dichloromethane (20 mL). The layers were separated and the aqueous fraction was extracted with dichloromethane (2×20 mL). The combined organics were dried, filtered and evaporated. Recrystallisation from acetonitrile gave the sub-title compound (140 mg).

MS: APCI(+ve) 482/484 (M+H⁺).

¹H NMR (400 MHz, d₆-DMSO) δ 8.35 (1H, t), 7.63 (1H, dd), 7.58 (1H, d), 7.51 (1H, d), 7.39-7.30 (2H, m), 7.12-7.01 (2H, m), 4.83 (2H, s), 4.15 (2H, q), 2.94 (2H, d), 1.93 (3H, s), 1.67 (3H, d), 1.59 (3H, d), 1.52 (6H, s), 1.20 (3H, t).

b) [[4′-Chloro-3′-[[(tricyclo[3.3.1.1^{3,7}]dec-1-ylmethyl)amino]carbonyl][1,1′-biphenyl]-2-yl]oxy]-acetic acid

A solution of potassium hydroxide (100 mg) in water (2 mL) was added to a solution of [[4′-chloro-3′-[[(tricyclo[3.3.1.1^{3,7}]dec-1-ylmethyl)amino]carbonyl][1,1′-biphenyl]-2-yl]oxy]-acetic acid, ethyl ester (Example 15 (a)) (140 mg) in methanol (2 mL) in a 10 mL microwave vial and this was heated at 70° C. for 5 minutes within a microwave. The reaction mixture was concentrated and then acidified to pH 5 using 2M aqueous hydrochloric acid. The solid was collected by filtration and purification (Varian NH₂ cartridge using methanol:dichloromethane 1:1 (100 mL) and then acetic acid:methanol:dichloromethane 1:10:10 (100 mL) as eluant) gave the title compound as a solid (55 mg).

MS: APCI(+ve) 454/456 (M+H⁺).

m.p. 208-210° C.

¹H NMR (400 MHz, d₆-DMSO) δ 8.36 (1H, t), 7.64 (1H, dd), 7.57 (1H, d), 7.51 (1H, d), 7.38-7.30 (2H, m), 7.06 (1H, t), 7.00 (1H, d), 4.70 (2H, s), 2.94 (2H, d), 1.93 (3H, s), 1.66 (3H, d), 1.59 (3H, d), 1.53 (6H, s).

### EXAMPLE 16

### 3-[[4′-Chloro-3′-[[(tricyclo[3.3.1.1^{3,7}]dec-1-ylmethyl)amino]carbonyl][1,1′-biphenyl]-2-yl]oxy]-propanoic acid

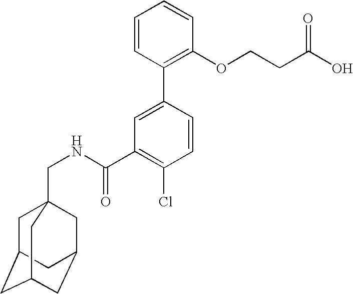

To a solution of 4-chloro-2′-hydroxy-N-(tricyclo[3.3.1.1^{3,7}]dec-1-ylmethyl)-[1,1′-biphenyl]-3-carboxamide (Prepared as described in Example 14 (a)) (200 mg) and potassium tert-butoxide (120 mg) in tetrahydrofuran (2 mL) was added β-propiolactone (0.06 mL). The reaction was stirred at room temperature under a nitrogen atmosphere for 16 hours then evaporated. The residue was suspended in deionised water (5 mL) and acidified to pH 5 with 2 M aqueous hydrochloric acid. The solid was collected by filtration and purified (Varian NH₂ cartridge using methanol (100 mL) and then 5% acetic acid in methanol (100 mL) as eluant). Further purification (SiO₂, dichloromethane:methanol 99:1 as eluant) afforded the title compound as a solid (10 mg).

MS: APCI(+ve) 468/470 (M+H⁺).

m.p. 163-164° C.

¹H NMR (400 MHz, d₆-DMSO) δ 8.36 (1H, t), 7.57 (1H, dd), 7.47-7.42 (2H, m), 7.40-7.33 (2H, m), 7.15 (1H, d), 7.06 (1H, t), 4.21 (2H, t), 2.95 (2H, d), 2.64 (2H, t), 1.94 (3H, s), 1.67 (3H, d), 1.59 (3H, d), 1.53 (6H, s).

### EXAMPLE 17

### 5-Chloro-2-[4-chloro-3-[[(tricyclo[3.3.1.1^{3,7}]dec-1-ylmethyl)amino]carbonyl]phenyl]-3-pyridinecarboxylic acid

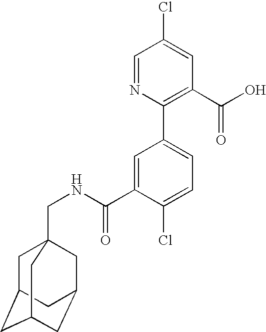

a) 2,5-Dichloro-3-pyridinecarboxylic acid, methyl ester

2,5-Dichloro-3-pyridinecarbonyl chloride (1.0 g) was added dropwise over 10 minutes to methanol (10 ml) with stirring under a nitrogen atmosphere. The resulting solution was stirred at room temperature for 16 hours and concentrated in vacuo to yield the sub-title compound as a colourless solid (0.95 g)

MS: APCI(+ve) 206 (M+H⁺).

b) 5-Chloro-2-[4-chloro-3-[[(tricyclo[3.3.1.1^{3,7}]dec-1-ylmethyl)amino]carbonyl]phenyl]-3-pyridinecarboxylic acid, methyl ester

Prepared according to the method of Example 1 using [4-chloro-3-[[(tricyclo[3.3.1.1^{3,7}]dec-1-ylmethyl)amino]carbonyl]phenyl]-boronic acid (Example 2 (a)) (300 mg), 2,5-dichloro-3-pyridinecarboxylic acid, methyl ester (Example 17 (a)) (180 mg), potassium carbonate (240 mg) and dichlorobis(triphenylphosphine)palladium (II) (50 mg) to give the sub-title compound as a colourless solid (350 mg).

MS: APCI(+ve) 475 (M+H⁺).

c) 5-Chloro-2-[4-chloro-3-[[(tricyclo[3.3.1.1^{3,7}]dec-1-ylmethyl)amino]carbonyl]phenyl]-3-pyridinecarboxylic acid

Prepared according to the method of Example 10 (b) using 5-chloro-2-[4-chloro-3-[[(tricyclo[3.3.1.1^{3,7}]dec-1-ylmethyl)amino]carbonyl]phenyl]-3-pyridinecarboxylic acid, methyl ester (Example 17 (b)) (350 mg). Purification (SiO₂, dichloromethane methanol 99:1, then dichloromethane:methanol:trifluoroacetic acid 98:1:1 as eluant) gave the title compound as a colourless solid (32 mg).

MS: APCI(+ve) 459 (M+H⁺).

m.p. 165-168° C.

¹H NMR(300 MHz, d₆-DMSO) δ 8.85 (1H, t), 8.44 (1H, t), 8.28 (1H, d), 7.61-7.55 (3H, m), 2.95 (2H, d), 1.94 (3H, s), 1.72-1.55 (6H, m), 1.52 (6H, s).

### EXAMPLE 18

### 4′-Chloro-6-methyl-3′-[[(tricyclo[3.3.1.1^{3,7}]dec-1-ylmethyl)amino]carbonyl]-[1,1′-biphenyl]-2-carboxylic acid

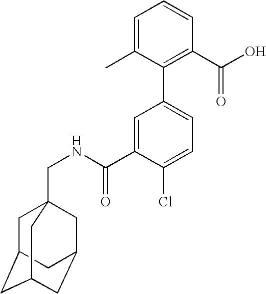

a) 2-Iodo-3-methyl-benzoic acid, methyl ester

Prepared according to the method of Example 13 (a) using 2-iodo-3-methyl-benzoic acid (1.0 g) to yield the sub-title compound as an oil (0.94 g).

MS: APCI(+ve) 276 (M+H⁺).

b) 4′-Chloro-6-methyl-3′-[[(tricyclo[3.3.1.1^{3,7}]dec-1-ylmethyl)amino]carbonyl]-[1,1′-biphenyl]-2-carboxylic acid, methyl ester

Prepared according to the method of Example 1 using [4-chloro-3-[[(tricyclo[3.3.1.1^{3,7}]dec-1-ylmethyl)amino]carbonyl]phenyl]-boronic acid (Example 2 (a)) (300 mg), 2-iodo-3-methyl-benzoic acid, methyl ester (Example 18 (a)) (250 mg), potassium carbonate (240 mg) and dichlorobis(triphenylphosphine)palladium (II) (50 mg) to give the sub-title compound as a solid (250 mg).

MS: APCI(+ve) 452 (M+H⁺).

c) 4′-Chloro-6-methyl-3′-[[(tricyclo[3.3.1.1^{3,7}]dec-1-ylmethyl)amino]carbonyl]-[1,1′-biphenyl]-2-carboxylic acid

Prepared according to the method of Example 10 (b) using 4′-Chloro-6-methyl-3′-[[(tricyclo[3.3.1.1^{3,7}]dec-1-ylmethyl)amino]carbonyl]-[1,1′-biphenyl]-2-carboxylic acid, methyl ester (Example 18 (b)) (250 mg). Purification (SiO₂, dichloromethane, then dichloromethane:methanol:trifluoroacetic acid 98:2:1 as eluant) gave the title compound as a colourless solid (42 mg).

MS: APCI(+ve) 438 (M+H⁺).

m.p. 151-154° C.

¹H NMR (400 MHz, d₆-DMSO) δ 12.60 (1H, s), 8.31 (1H, t), 7.61 (1H, d), 7.49 (1H, d), 7.48 (1H, d), 7.38 (1H, dd), 7.22 (1H, dd), 7.13 (1H, d), 2.93 (2H, d), 2.08 (3H, s), 1.93 (3H, s), 1.70-1.55 (6H, m), 1.52 (6H, s).

### EXAMPLE 19

### 3-[4-Chloro-3-[[(tricyclo[3.3.1.1^{3,7}]dec-1-ylmethyl)amino]carbonyl]phenyl]-2-thiophenecarboxylic acid

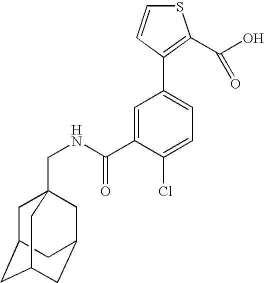

a) 3-[4-chloro-3-[[(tricyclo[3.3.1.1^{3,7}]dec-1-ylmethyl)amino]carbonyl]phenyl]-2-thiophenecarboxylic acid, methyl ester

Prepared according to the method of Example 1, using [4-chloro-3-[[(tricyclo[3.3.1.1^{3,7}]dec-1-ylmethyl)amino]carbonyl]phenyl]-boronic acid (Example 2 (a)) (0.3 g), 3-bromo-2-thiophenecarboxylic acid, methyl ester (0.19 g), potassium carbonate (240 mg) and dichlorobis(triphenylphosphine)palladium (II) (25 mg). The mixture was filtered through diatomaceous earth, washing with methanol (2×30 ml) and was concentrated in vacuo. The residue was partitioned between dichloromethane (30 ml) and deionised water (10 ml), the layers were separated and the aqueous fraction was extracted with dichloromethane (2×30 ml). The combined organic layers were dried (MgSO₄), evaporated and purified (SiO₂, 85:15 isohexane:ethyl acetate to give the sub-title compound as a colourless solid (160 mg).

MS: APCI(+ve) 444 (M+H⁺).

b) 3-[4-Chloro-3-[[(tricyclo[3.3.1.1^{3,7}]dec-1-ylmethyl)amino]carbonyl]phenyl]-2-thiophenecarboxylic acid

Prepared according to the method of Example 10 (b) using 3-[4-chloro-3-[[(tricyclo[3.3.1.1^{3,7}]dec-1-ylmethyl)amino]carbonyl]phenyl]-2-thiophenecarboxylic acid, methyl ester (Example 19 (a)) (160 mg). Purification (SiO₂, dichloromethane, then dichloromethane:methanol:trifluoroacetic acid 98:2:1 as eluant, then RP-HPLC, acetonitrile:aqueous ammonium acetate, Symmetry) gave the title compound as a colourless solid (47 mg).

MS: APCI(+ve) 430 (M+H⁺).

m.p. 140-143° C.

¹H NMR (400 MHz, d₆-DMSO) δ 8.34 (1H, t), 7.71 (1H, d), 7.59-7.54 (1H, m), 7.49-7.43 (2H, m), 7.15 (1H, d), 2.94 (2H, d), 1.93 (3H, s), 1.70-1.56 (6H, m), 1.52 (6H, s).

### EXAMPLE 20

### 6-[4-Chloro-3-[[(tricyclo[3.3.1.1^{3,7}]dec-1-ylmethyl)amino]carbonyl]phenyl]-2-pyridinecarboxylic acid

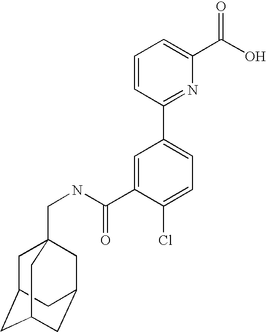

a) 6-[4-Chloro-3-[[(tricyclo[3.3.1.1^{3,7}]dec-1-ylmethyl)amino]carbonyl]phenyl]-2-pyridinecarboxylic acid, methyl ester

Prepared according to the method of Example 10 (a) using [4-chloro-3-[[(tricyclo[3.3.1.1^{3,7}]dec-1-ylmethyl)amino]carbonyl]phenyl]-boronic acid (Example 2 (a)) (250 mg) and 6-chloro-2-pyridinecarboxylic acid, methyl ester (150 mg). Purification (SiO₂, ethyl acetate:isohexane 25:75 as eluent) afforded the sub-title compound as a solid (120 mg).

¹H NMR (400 MHz, d₆-DMSO) δ 8.46 (1H, t), 8.32 (1H, dd), 8.22-8.16 (2H, m), 8.12 (1H, t), 8.05 (1H, dd), 7.65 (1H, d), 3.92 (3H, s), 2.98 (2H, d), 1.96 (3H, s), 1.69 (3H, d), 1.62 (3H, d), 1.56 (6H, s).

MS: APCI(+ve) 439/441 (M+H⁺).

b) 6-[4-Chloro-3-[[(tricyclo[3.3.1.1^{3,7}]dec-1-ylmethyl)amino]carbonyl]phenyl]-2-pyridinecarboxylic acid

Prepared according to the method of Example 10 (b) using 6-[4-chloro-3-[[(tricyclo[3.3.1.1^{3,7}]dec-1-ylmethyl)amino]carbonyl]phenyl]-2-pyridinecarboxylic acid methyl ester (Example 20 (a)) (120 mg). Purification (Varian NH₂ cartridge using methanol (100 mL) and then acetic acid:methanol 1:20 (100 mL) as eluant) afforded the title compound as a solid (65 mg).

¹H NMR (400 MHz, d₆-DMSO) δ 8.45 (1H, s), 8.26-8.14 (3H, m), 8.03 (1H, t), 7.96 (1H, d), 7.62 (1H, d), 2.99 (2H, d), 1.95 (3H, s), 1.75-1.48 (12H, m).

MS: APCI(+ve) 425/427 (M+H⁺).

m.p. 170-173° C.

### EXAMPLE 21

### 3-[4-Chloro-3-[[(tricyclo[3.3.1.1^{3,7}]dec-1-ylmethyl)amino]carbonyl]phenyl]-2-pyridinecarboxylic acid

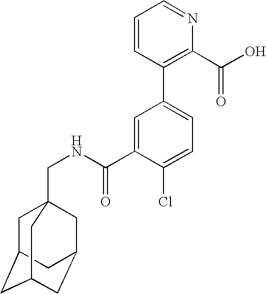

a) 2-Chloro-5-(2-chloro-3-pyridinyl)-N-(tricyclo [3.3.1.1^{3,7}]dec-1-ylmethyl)-benzamide

[4-Chloro-3-[[(tricyclo[3.3.1.1^{3,7}]dec-1-ylmethyl)amino]carbonyl]phenyl]-boronic acid (Example 2 (a)) (3.1 g) was added to a solution of 3-bromo-2-chloropyridine (1.7 g) in tetrahydrofuran (30 mL). A solution of potassium carbonate (2.4 g) in water (30 mL) was added followed by dichlorobis(triphenylphosphine)palladium (II) (0.25 g) and the mixture was stirred under a nitrogen atmosphere at room temperature for 3 hours. The reaction mixture was concentrated and the residue partitioned between dichloromethane and water. The layers were separated and the organic fraction was dried (MgSO₄), filtered and concentrated. Purification by chromatography (SiO₂, 70:30 isohexane:ethyl acetate, then 60:40 isohexane:ethyl acetate as eluant) gave the sub-title compound as a colourless solid (1.85 g)

MS: APCI(+ve) 415/417 (M+H⁺).

b) 2-Chloro-5-(2-cyano-3-pyridinyl)-N-(tricyclo [3.3.1.1^{3,7}]dec-1-ylmethyl)-benzamide

A mixture of 2-chloro-5-(2-chloro-3-pyridinyl)-N-(tricyclo[3.3.1.1^{3,7}]dec-1-ylmethyl)-benzamide (Example 21 (a)) (0.25 g), bis(dibenzylideneacetone)palladium (0.12 g), 1,1′-bis(diphenylphosphino)ferrocene (0.29 g) and copper (I) cyanide (0.34 g) in 1,4-dioxane (3 mL) was heated at 130° C. in a microwave for 2 hours. The reaction was concentrated to dryness and partitioned between dichloromethane and water. The organic fraction was dried (MgSO₄), filtered and concentrated to dryness before being purified by chromatography (SiO₂, 1:1 ethyl acetate:isohexane as eluant) to give the sub-title compound as a foam (0.20 g)

MS: APCI(+ve) 406/408 (M+H⁺).

c) 3-[4-Chloro-3-[[(tricyclo[3.3.1.1^{3,7}]dec-1-ylmethyl)amino]carbonyl]phenyl]-2-pyridinecarboxylic acid

A mixture of 2-chloro-5-(2-cyano-3-pyridinyl)-N-(tricyclo[3.3.1.1^{3,7}]dec-1-ylmethyl)-benzamide (0.20 g) (Example 21 (b)) and potassium hydroxide (0.17 g) in water (1 mL) and ethanol (10 mL) was heated at reflux for 16 hours. The reaction mixture was then concentrated to dryness and the residue redissolved in water (2 mL) and concentrated hydrochloric acid (2 mL). This mixture was then heated at reflux for 5 hours, allowed to cool to room temperature and was purified by RP-HPLC (acetonitrile:aqueous ammonium acetate, Symmetry) to give the title compound as a solid (99 mg).

MS: APCI(+ve) 425/427 (M+H⁺).

¹H NMR (400 MHz, d₆-DMSO) δ 8.38-833 (2H, m), 7.72-7.65 (2H, m), 7.57 (1H, d), 7.46 (1H, d), 7.24 (1H, dd), 2.95 (2H, d), 1.94 (3H, s), 1.66 (3H, d), 1.60 (3H, d), 1.53 (6H, s).

### EXAMPLE 22

### 2-Chloro-5-[2-(1H-tetrazol-5-yl)-3-pyridinyl]-N-(tricyclo[3.3.1.1^{3,7}]dec-1-ylmethyl)-benzamide

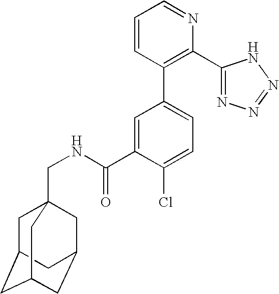

Prepared according to the method of Example 9 using 2-chloro-5-(2-cyano-3-pyridinyl)-N-(tricyclo[3.3.1.1^{3,7}]dec-1-ylmethyl)-benzamide (Example 21 (b)) (0.13 g). Purification (Varian NH₂ cartridge using methanol and then 1% trifluoroacetic acid in methanol as eluant) followed by RP-HPLC (acetonitrile:aqueous trifluoroacetic acid, Symmetry) gave the title compound as a solid (36 mg).

MS: APCI(+ve) 449 (M+H⁺).

¹H NMR (d₆-DMSO, 300 MHz) δ 8.84-8.82 (1H, d), 8.36-8.32 (1H, t), 8.02-7.99 (1H, d), 7.76-7.72 (1H, m), 7.49-7.46 (1H, d), 7.36-7.29 (2H, m), 2.93-2.91 (2H, d), 1.93 (3H, s), 1.68-1.57 (6H, q), 1.50 (6H, s).

### EXAMPLE 23

### 2-[4-Chloro-3-[[(tricyclo[3.3.1.1^{3,7}]dec-1-ylmethyl)amino]carbonyl]phenyl]-4-oxazolecarboxylic acid

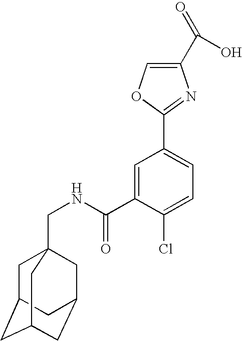

a) 4-Chloro-3-[[(tricyclo[3.3.1.1^{3,7}]dec-1-ylmethyl)amino]carbonyl]-benzoic acid

Potassium peroxomonosulfate (1.9 g) was added to a solution of 2-chloro-5-formyl-N-(tricyclo[3.3.1.1^{3,7}]dec-1-ylmethyl)-benzamide (Prepared as described in WO200144170) (1.0 g) in N,N-dimethylformamide (15 mL) and the mixture was stirred at room temperature for 5 hours. Ethyl acetate (50 mL) and aqueous hydrochloric acid (20 mL, 1M) were added, the layers were separated and the aqueous fraction was extracted with ethyl acetate (50 mL). The combined organics were washed with water (30 mL) and saturated aqueous sodium chloride (30 mL) before being dried (MgSO₄), filtered and concentrated to give the sub-title compound as a colourless solid (1.0 g).

MS: APCI(+ve) 348/350 (M+H⁺).

b) N-[4-Chloro-3-[[(tricyclo[3.3.1.1^{3,7}]dec-1-ylmethyl)amino]carbonyl]benzoyl]-L-serine, methyl ester

1-Hydroxybenzotriazole (0.88 g) and 1-(3-dimethylaminopropyl)-3-ethylcarbodiimide hydrochloride (1.1 g) were added to a stirred solution of 4-chloro-3-[[(tricyclo[3.3.1.1^{3,7}]dec-1-ylmethyl)amino]carbonyl]-benzoic acid (Example 23 (a)) (1.0 g) and triethylamine (1.6 mL) in dichloromethane (15 mL) under nitrogen at 0° C. L-Serine methyl ester hydrochloride (0.90 g) was added and the mixture was allowed to warm to room temperature over 16 hours. Water (20 mL) was added, the layers were separated and the aqueous fraction was extracted with dichloromethane (2×20 mL). The combined organic layers were washed with aqueous hydrochloric acid (20 mL, 2M) and saturated aqueous sodium chloride (20 mL) before being dried (MgSO₄), filtered and concentrated to yield the sub-title compound as a colourless solid (1.1 g).

MS: APCI(+ve) 449/451 (M+H⁺).

c) 2-[4-Chloro-3-[[(tricyclo[3.3.1.1^{3,7}]dec-1-ylmethyl)amino]carbonyl]phenyl]-4-oxazolecarboxylic acid, methyl ester

Diethylaminosulfur trifluoride (0.41 mL) was added dropwise over 10 minutes to a stirred solution of N-[4-chloro-3-[[(tricyclo[3.3.1.1^{3,7}]dec-1-ylmethyl)amino]carbonyl]benzoyl]-L-serine, methyl ester (Example 23 (b)) (1.0 g) in dichloromethane (15 mL) at −78° C. under nitrogen. The mixture was stirred at −78° C. for 1 hour and was then allowed to warm to −30° C. Saturated aqueous sodium hydrogen carbonate (5 mL) was added, the mixture was allowed to warm to room temperature and the layers were separated. The aqueous fraction was extracted with dichloromethane (2×10 mL), the combined organic layers were combined and then dried (MgSO₄), filtered and concentrated before being purified by chromatography (SiO₂, dichloromethane as eluant). The material was dissolved in dichloromethane (15 mL), cooled to 0° C. and bromotrichloromethane (0.44 mL) was added dropwise. The mixture was stirred under nitrogen for 15 minutes, 1,8-diazabycyclo[5.4.0]undec-7-ene (0.67 mL) was added dropwise and the mixture was allowed to warm to room temperature over 16 hours. Water (10 mL) was added, the layers separated and the aqueous fraction was extracted with dichloromethane (2×15 mL). The combined organic layers were washed with aqueous hydrochloric acid (10 mL, 2M) and saturated aqueous sodium chloride (10 mL) before being dried (MgSO₄), filtered and concentrated to leave the sub-title product (0.91 g).

MS: APCI(+ve) 429 (M+H⁺).

d) 2-[4-Chloro-3-[[(tricyclo[3.3.1.1^{3,7}]dec-1-ylmethyl)amino]carbonyl]phenyl]-4-oxazolecarboxylic acid

Sodium hydroxide (75 mg) in water (2 mL) was added to a stirred solution of 2-[4-chloro-3-[[(tricyclo[3.3.1.1^{3,7}]dec-1-ylmethyl)amino]carbonyl]phenyl]-4-oxazolecarboxylic acid, methyl ester (Example 23 (c)) (400 mg) in tetrahydrofuran (5 mL). The reaction was stirred at room temperature for 24 hours. The mixture was concentrated, acidified with aqueous hydrochloric acid (2M) and the resulting precipitate was collected by filtration, washing with water (2×10 mL) and methanol (10 mL) to give the title compound as a solid (86 mg).

MS: APCI(+ve) 415/417 (M+H⁺).

m.p. 254-257° C.

¹H NMR (400 MHz, d₆-DMSO) δ 8.62 (1H, s), 8.48 (1H, s), 8.02 (1H, dd), 7.99 (1H, d), 7.67 (1H, d) 2.99 (2H, d), 1.91 (3H, m), 1.73-1.57 (6H, m), 1.57-1.51 (6H, m).

### EXAMPLE 24

### 4′-Chloro-4-methyl-3′-[[(tricyclo[3.3.1.1^{3,7}]dec-1-ylmethyl)amino]carbonyl]-[1,1′-biphenyl]-2-carboxylic acid

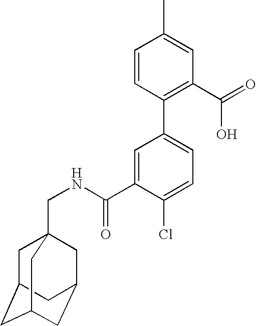

a) 2-Iodo-5-methyl-benzoic acid, methyl ester

Prepared according to the method of Example 13 (a) using 2-iodo-5-methyl-benzoic acid (1.0 g) to give the sub-title compound as a colourless oil (1.0 g)

MS: APCI(+ve) 276 (M+H⁺).

b) 4′-Chloro-5-methyl-3′-[[(tricyclo[3.3.1.1^{3,7}]dec-1-ylmethyl)amino]carbonyl]-[1,1′-biphenyl]-3-carboxylic acid, methyl ester

Prepared according to the method of Example 14 (a) using [4-chloro-3-[[(tricyclo[3.3.1.1^{3,7}]dec-1-ylmethyl)amino]carbonyl]phenyl]-boronic acid (Example 2 (a)) (300 mg) and 2-iodo-5-methyl-benzoic acid, methyl ester (Example 24 (a)) (240 mg) at room temperature. Purification (SiO₂, ethyl acetate:isohexane 15:85 as eluent) afforded the sub-title compound as a colourless solid (160 mg).

MS: APCI(+ye) 452 (M+H⁺).

c) 4′-Chloro-4-methyl-3′-[[(tricyclo[3.3.1.1^{3,7}]dec-1-ylmethyl)amino]carbonyl]-[1,1′-biphenyl]-2-carboxylic acid

Prepared according to the method of Example 23 (d), using 4′-chloro-5-methyl-3′-[[(tricyclo[3.3.1.1^{3,7}]dec-1-ylmethyl)amino]carbonyl]-[l, 1,1′-biphenyl]-3-carboxylic acid, methyl ester (Example 24 (b)) (160 mg). The mixture was concentrated and acidified with aqueous hydrochloric acid (2M) and the residue was purified firstly by chromatography. (SiO₂, dichloromethane, then 99:1 dichloromethane methanol as eluant), and then by recrystallisation from acetonitrile:water to give the title compound as a solid (24 mg).

MS: APCI(+ve) 438/440 (M+H⁺).

m.p. 214-216° C.

¹H NMR (400 MHz, d₆-DMSO) δ 12.88 (1H, s), 8.37 (1H, t), 7.59 (1H, d), 7.49 (1H, d), 7.42 (1H, dd), 7.34 (1H, dd), 7.31 (1H, d), 7.30 (1H, d), 2.94 (2H, d), 2.38 (3H, s), 1.94 (3H, s), 1.70-1.56 (6H, m), 1.52 (6H, d).

### EXAMPLE 25

### 6-[4-Chloro-3-[[(tricyclo[3.3.1.1^{3,7}]dec-1-ylmethyl)amino]carbonyl]phenyl]-N-(methylsulfonyl)-2-pyridinecarboxamide

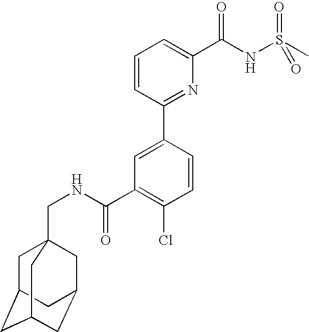

To a stirred mixture of 6-[4-chloro-3-[[(tricyclo[3.3.1.1^{3,7}]dec-1-ylmethyl)amino]carbonyl]phenyl]-2-pyridinecarboxylic acid (Example 20) (250 mg) and methanesulfonamide (57 mg) in dichloromethane (3 mL) was added 4-(dimethylamino)pyridine (73 mg) and l-(3-dimemylaminopropyl)-3-ethylcarbodiimide hydrochloride (115 mg). The reaction was stirred at room temperature under nitrogen for 16 hours. Dichloromethane (100 mL) was then added and the solution was washed with water (50 ml) and aqueous hydrochloric acid (2×50 mL, 2M) before being dried (MgSO₄), filtered and evaporated. Purification by chromatography (SiO₂, dichloromethane-.methanol 98:2 as eluant) and then further purification (Varian NH₂ cartridge using 1:1 dichloromethane:methanol (100 mL) and then 1:10:10 acetic acid:dichloromethane:methanol (100 mL) as eluant) afforded the title compound as a solid (190 mg).

MS: APCI(+ve) 502/504 (M+H⁺).

m.p. 160-162° C.

¹H NMR (400 MHz, d₆-DMSO) δ 8.46 (1H, t), 8.42-8.36 (2H, m), 8.32 (1H, d), 8.14 (1H, t), 8.05 (1H, d), 7.64 (1H, d), 3.38 (3H, s), 2.99 (2H, d), 1.96 (3H, s), 1.68 (3H, d), 1.61 (3H, d), 1.56 (6H, s).

### EXAMPLE 26

### N-[3-[4-Chloro-3-[[(tricyclo[3.3.1.1^{3,7}]dec-1-ylmethyl)amino]carbonyl]phenyl]-2-pyridinyl]-glycine

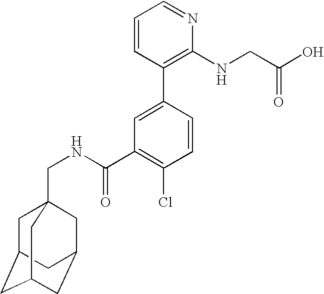

a) 5-(2-Amino-3-pyridinyl)-2-chloro-N-(tricyclo[3.3.1.1^{3,7}]dec-1-ylmethyl)-benzamide

To a stirred mixture of [4-chloro-3-[[(tricyclo[3.3.1.1^{3,7}]dec-1-ylmethyl)amino]carbonyl]phenyl]-boronic acid (Example 2 (a)) (1.0 g) and 2-amino-3-bromopyridine (640 mg) in toluene (10 mL) and ethanol (10 mL) was added a solution of sodium carbonate (610 mg) in water (10 mL), followed by tetrakis(triphenylphosphine)palladium(0) (140 mg). The mixture was heated to 50° C. for 2 hours, then allowed to cool and was concentrated. The residue was partitioned between dichloromethane (100 mL) and water (100 mL), the layers were separated and the aqueous was extracted with dichloromethane (2×50 mL). The combined organics were evaporated. Purification by chromatography (SiO₂, 3:1 isohexane:ethyl acetate as the eluant) gave the sub-title compound as a solid (500 mg).

MS: APCI(+ve) 396/398 (M+H⁺).

¹H NMR (400 MHz, d₆-DMSO) δ 8.31 (1H, t), 7.98 (1H, dd), 7.66-7.52 (1H, m), 7.48-7,44 (2H, m), 7.37 (1H, dd), 6.67 (1H, dd), 5.77 (2H, s), 2.96 (2H, d), 1.94 (3H, s), 1.67 (3H, d), 1.60 (3H, d), 1.52 (6H, s).

b) N-[3-[4-Chloro-3-[[(tricyclo[3.3.1.1^{3,7}]dec-1-ylmethyl)amino]carbonyl]phenyl]-2-pyridinyl]-glycine 1,1-dimethylethyl ester

5-(2-Amino-3-pyridinyl)-2-chloro-N-(tricyclo[3.3.1.1^{3,7}]dec-1-ylmethyl)-benzamide (Example 26 (a)) (300 mg), tert-butylbromoacetate (0.5 mL) and acetonitrile (2 mL) were placed in a 10 mL vial and heated at 70° C. for 2 hours in a microwave. The mixture was then concentrated and subsequent purification by chromatography (SiO₂, dichloromethane methanol 95:5 as eluant) afforded the sub-title compound as a solid (200 mg).

MS: APCI(+ve) 510/512 (M+H⁺).

c) N-[3-[4-Chloro-3-[[(tricyclo[3.3.1.1^{3,7}]dec-1-ylmethyl)amino]carbonyl]phenyl]-2-pyridinyl]-glycine

To a solution of N-[3-[4-chloro-3-[[(tricyclo[3.3.1.1^{3,7}]dec-1-ylmethyl)amino]carbonyl]phenyl]-2-pyridinyl]-glycine 1,1-dimethylethyl ester (Example 26 (b)) (200 mg) in dichloromethane (3 mL) was added trifluoroacetic acid (1 mL). The mixture was stirred at room temperature for 2 hours and then poured into saturated aqueous sodium bicarbonate (100 mL). This was washed with dichloromethane (3×50 mL) and the aqueous fraction was filtered to give a solid which was purified by chromatography (SiO₂, dichloromethane:methanol 90:10 as eluant) to afford the title compound as a solid (40 mg).

MS: APCI(+ve) 454/456 (M+H⁺).

m.p. 160-164° C.

¹H NMR (300 MHz, d₆-DMSO) δ 8.25 (1H, t), 8.03 (1H, dd), 7.76 (1H, dd), 7.66 (1H, d), 7.52-7.41 (2H, m), 6.98 (1H, t), 4.69 (2H, s), 2.97 (2H, d), 1.94 (3H, s), 1.67 (3H, d), 1.59 (3H, d), 1.52 (6H, s).

### EXAMPLE 27

### 2-Chloro-5-[6-[(methylsulfonyl)amino]-2-pyridinyl]-N-(tricyclo[3.3.1.1^{3,7}]dec-1-ylmethyl)-benzamide

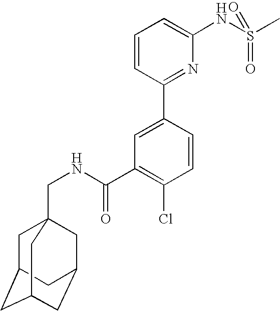

a) 2-Chloro-5-(6-chloro-2-pyridinyl)-N-(tricyclo[3.3.1.1^{3,7}]dec-1-ylmethyl)-benzamide

Prepared according to the method of Example 14 (a) using [4-chloro-3-[[(tricyclo[3.3.1.1^{3,7}]dec-1-ylmethyl)amino]carbonyl]phenyl]-boronic acid (Example 2 (a)) (2.5 g) and 2,6-dichloropyridine (4.3 g) at room temperature. Purification (SiO₂, 9:1 isohexane:ethyl acetate as the eluant) gave the sub-title compound as a solid (1.1 g).

MS: APCI(+ve) 415/417 (M+H⁺).

¹H NMR (300 MHz, CDCl₃) δ 8.29 (1H, d), 8.07 (1H, dd), 7.78-7.65 (2H, m), 7.51 (1H, d), 7.30 (1H, dd), 6.31 (1H, s), 3.21 (2H, d), 2.02 (3H, s), 1.80-1.59 (12H, m).

b) 2-Chloro-5-[6-[(methylsulfonyl)amino]-2-pyridinyl]-N-(tricyclo[3.3.1.1^{3,7}]dec-1-ylmethyl)-benzamide

2-Chloro-5-(6-chloro-2-pyridinyl)-N-(tricyclo [3.3.1.1^{3,7}]dec-1-ylmethyl)-benzamide (Example 27 (a)) (400 mg), methanesulfonamide (92 mg) and potassium carbonate (130 mg) were dissolved in methyl sulfoxide (3 mL). The mixture was heated to 190° C. under nitrogen for 5 hours, then allowed to cool. Purification by chromatography (SiO₂, 3:1 isohexane:ethyl acetate as eluant) and then by RP-HPLC (acetonitrile:aqueous ammonium acetate, Symmetry) afforded the title compound as a solid (19 mg).

MS: APCI(+ve) 474/476 (M+H⁺).

m.p. 115-120° C.

¹H NMR (400 MHz, d₆-DMSO) δ 8.43 (1H, t), 8.11-8.03 (2H, m), 7.80 (1H, t), 7.67-7.58 (2H, m), 6.88 (1H, d), 3.34 (3H, s), 2.97 (2H, d), 1.95 (3H, s), 1.68 (3H, d), 1.62 (3H, d), 1.55 (6H, s).

### EXAMPLE 28

### [[3-[4-Chloro-3-[[(tricyclo[3.3.1.1^{3,7}]dec-1-ylmethyl)amino]carbonyl]phenyl]-2-pyridinyl]oxy]-acetic acid

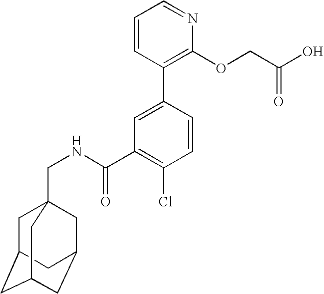

a) [[3-[4-Chloro-3-[[(tricyclo[3.3.1.1^{3,7}]dec-1-ylmethyl)amino]carbonyl]phenyl]-2-pyridinyl]oxy]-acetic acid methyl ester

2-Chloro-5-(2-chloro-3-pyridinyl)-N-(tricyclo[3.3.1.1^{3,7}]dec-1-ylmethyl)-benzamide (Example 21 (a)) (100 mg), methyl glycolate (110 mg) and potassium tert-butoxide (1 mL, 1M solution in tetrahydrofuran) were placed in a 10 mL vial and heated at 70° C. for 30 minutes in a microwave. The reaction mixture was allowed to cool and was concentrated. Purification by chromatography (SiO₂, 3:1 isohexane:ethyl acetate as the eluant) afforded the sub-title compound as a solid (70 mg).

MS: APCI(+ve) 469/471 (M+H⁺).

b) [[3-[4-Chloro-3-[[(tricyclo[3.3.1.1^{3,7}]dec-1-ylmethyl)amino]carbonyl]phenyl]-2-pyridinyl]oxy]-acetic acid

[[3-[4-Chloro-3-[[(tricyclo[3.3.1.1^{3,7}]dec-1-ylmethyl)amino]carbonyl]phenyl]-2-pyridinyl]oxy]-acetic acid methyl ester (Example 28 (a)) (70 mg) and methanol (1 mL) were placed in a 10 ml vial. A solution of potassium hydroxide (100 mg) in water (1 mL) was added and the mixture was heated at 50° C. for 5 minutes in a microwave. The mixture was concentrated, water (10 mL) was added to the residue and this was then acidified to pH 2 with 2M aqueous hydrochloric acid. The resulting precipitate was collected by filtration. Purification (Varian NH₂ cartridge using dichloromethane (100 mL) and then 5% trifluoroacetic acid in dichloromethane (100 mL) as eluant) afforded the title compound as a solid (17 mg).

MS: APCI(−ve) 453/455 (M−H⁺).

m.p. 226-229° C.

¹H NMR (400 MHz, d₆-DMSO) δ 8.40 (1H, t), 8.15 (1H, dd), 7.85 (1H, dd), 7.71 (1H, dd), 7.66 (1H, d), 7.57 (1H, d), 7.15 (1H, dd), 4.87 (2H, s), 2.95 (2H, d), 1.92 (3H, d), 1.67 (3H, d), 1.59 (3H, d), 1.53 (6H, s).

### EXAMPLE 29

### 2-Chloro-5-[3-(1H-tetrazol-5-ylmethoxy)-2-pyridinyl]-N-(tricyclo[3.3.1.1^{3,7}]dec-1-ylmethyl)-benzamide

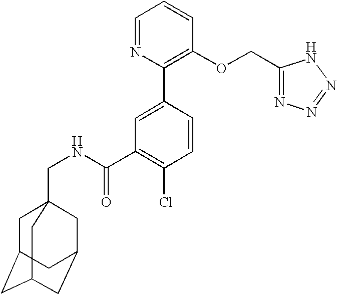

a) 2-Chloro-5-(3-hydroxy-2-pyridinyl)-N-(tricyclo[3.3.1.1^{3,7}]dec-1-ylmethyl)-benzamide

Prepared according to the method of Example 14 (a) using [4-chloro-3-[[(tricyclo[3.3.1.1^{3,7}]dec-1-ylmethyl)amino]carbonyl]phenyl]-boronic acid (Example 2 (a)) (2.0 g) and 2-bromo-3-hydroxypyridine (1.0 g) at room temperature to afford the sub-title compound as a solid (900 mg).

MS: APCI(+ve) 397/399 (M+H⁺).

¹H NMR (400 MHz, d₆-DMSO) δ 10.38 (1H, s), 8.38 (1H, t), 8.17 (1H, dd), 8.14-8.09 (2H, m), 7.53 (1H, d), 7.36 (1H, dd), 7.24 (1H, dd), 2.96 (2H, d), 1.95 (3H, s), 1.68 (3H, d), 1.60 (3H, d), 1.54 (6H, s).

b) 2-Chloro-5-[3-(cyanomethoxy)-2-pyridinyl]-N-(tricyclo[3.3.1.1^{3,7}]dec-1-ylmethyl)-benzamide

To a solution of 2-chloro-5-(3-hydroxy-2-pyridinyl)-N-(tricyclo[3.3.1.1^{3,7}]dec-1-ylmethyl)-benzamide (Example 29 (a)) (350 mg) and potassium carbonate (240 mg) in acetone (4 mL) was added chloroacetonitrile (0.1 mL). The mixture was heated at 55° C. for 6 hours and was then allowed to cool and was concentrated. Purification by chromatography (SiO₂, 3:1 isohexane:ethyl acetate as eluant) afforded the sub-title compound as a solid (200 mg).

MS: APCI(+ve) 436/438 (M+H⁺).

c) 2-Chloro-5-[3-(1H-tetrazol-5-ylmethoxy)-2-pyridinyl]-N-(tricyclo[3.3.1.1^{3,7}]dec-1-ylmethyl)-benzamide

Prepared according to the method of Example 9 using 2-chloro-5-[3-(cyanomethoxy)-2-pyridinyl]-N-(tricyclo[3.3.1.1^{3,7}]dec-1-ylmethyl)-benzamide (Example 29 (b)) (200 mg). Purification (Varian NH₂ cartridge using 1:1 dichloromethane:methanol (100 mL) and then 1:5:5 acetic acid:dichloromethane:methanol (100 mL) as eluant) and further purification by RP-HPLC (acetonitrile:aqueous trifluoroacetic acid, Symmetry) afforded the title compound as a solid (69 mg).

MS: APCI(+ve) 479/481 (M+H⁺).

m.p. 122-126° C.

¹H NMR (400 MHz, d₆-DMSO) δ 8.38-8.30 (2H, m), 7.97 (1H, dd), 7.92 (1H, d), 7.74 (1H, d), 7.52 (1H, d), 7.44 (1H, dd), 5.63 (2H, s), 2.94 (2H, d), 1.93 (3H, s), 1.66 (3H, d), 1.58 (3H, d), 1.51 (6H, s).

### EXAMPLE 30

### 4′-Chloro-4-methoxy-3′-[[(tricyclo[3.3.1.1^{3,7}]dec-1-ylmethyl)amino]carbonyl]-[1,1′-biphenyl]-2-carboxylic acid

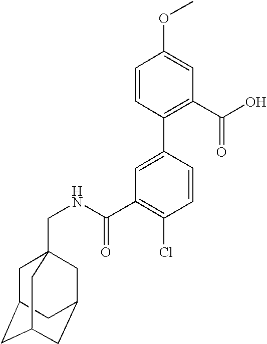

a) 2-Bromo-5-methoxy-benzoic acid ethyl ester

Prepared according to the method of Example 13 (a) using 2-bromo-5-methoxybenzoic acid (250 mg) and ethanol (2 mL) to afford the sub-title compound as a solid (280 mg).

MS: APCI(−ve) 258/260 (M−H⁺).

b) 4′-Chloro-4-methoxy-3′-[[(tricyclo[3.3.1.1^{3,7}]dec-1-ylmethyl)amino]carbonyl]-[1,1′-biphenyl]-2-carboxylic acid ethyl ester

Prepared according to the method of Example 14 (a) using [4-chloro-3-[[(tricyclo[3.3.1.1^{3,7}]dec-1-ylmethyl)amino]carbonyl]phenyl]-boronic acid (Example 2 (a)) (250 mg) and 2-bromo-5-methoxy-benzoic acid ethyl ester (Example 30 (a)) (280 mg) at room temperature to afford the sub-title compound as a solid (165 mg).

MS: APCI(+ve) 482/484 (M+H⁺).

¹H NMR (400 MHz, d₆-DMSO) δ 8.35 (1H, t), 7.50 (1H, d), 7.39 (1H, d), 7.32 (1H, dd), 7.27 (1H, d), 7.26-7.19 (2H, m), 4.09 (2H, q), 3.84 (3H, s), 2.94 (2H, d), 1.94 (3H, s), 1.67 (3H, d), 1.59 (3H, d), 1.52 (6H, s), 1.05 (3H, t).

c) 4′-Chloro-4-methoxy-3′-[[(tricyclo[3.3.1.1^{3,7}]dec-1-ylmethyl)amino]carbonyl]-[1,1′-biphenyl]-2-carboxylic acid

Prepared according to the method of Example 28 (b) using 4′-chloro-4-methoxy-3′-[[(tricyclo[3.3.1.1^{3,7}]dec-1-ylmethyl)amino]carbonyl]-[1,1′-biphenyl]-2-carboxylic acid ethyl ester (Example 30 (b)) (165 mg) to afford the title compound as a solid (75 mg).

MS: APCI(+ve) 454/456 (M+H⁺).

m.p. 219-221° C.

¹H NMR (400 MHz, d₆-DMSO) δ 8.35 (1H, t), 7.46 (1H, d), 7.38-7.27 (3H, m), 7.22 (1H, d), 7.12 (1H, dd), 3.82 (3H, s), 2.94 (2H, d), 1.93 (3H, s), 1.66 (3H, d), 1.59 (3H, d), 1.52 (6H, s).

### EXAMPLE 31

### 4-[4-Chloro-3-[[(tricyclo[3.3.1.1^{3,7}]dec-1-ylmethyl)amino]carbonyl]phenyl]-1-methyl-1H-pyrazole-3-carboxylic acid

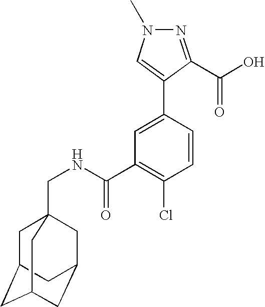

a) 2-Chloro-5-(3-formyl-1-methyl-1H-pyrazol-4-yl)-N-(tricyclo[3.3.1.1^{3,7}]dec-1-ylmethyl)-benzamide

A mixture of [[(tricyclo[3.3.1.1^{3,7}]dec-1-ylmethyl)amino]carbonyl]phenyl]-boronic acid (Example 2 (a)) (300 mg), 4-bromo-1-methyl-1H-pyrazole-3-carboxaldehyde (160 mg), potassium carbonate (240 mg) and tetrakis(triphenylphosphine)palladium(0) (100 mg) in 1,2-dimethoxyethane (5 mL)/water (5 mL) was heated at 80° C. with stirring under a nitrogen atmosphere for 3 hours. The products were filtered through diatomaceous earth, washing with methanol (3×10 mL), and the volatile components were removed in vacuo. The residue was partitioned between dichloromethane (50 mL) and water (20 mL), the layers were separated and the aqueous fraction was extracted with dichloromethane (2×25 mL). The combined organic layers were washed with 2M aqueous hydrochloric acid (25 mL), saturated aqueous sodium hydrogen carbonate (25 mL) and saturated aqueous sodium chloride (25 mL) before being dried (MgSO₄), filtered and concentrated to give the sub-title compound as a solid (310 mg).

MS: APCI(+ve) 412/414 (M+H⁺).

b) 4-[4-Chloro-3-[[(tricyclo[3.3.1.1^{3,7}]dec-1-ylmethyl)amino]carbonyl]phenyl]-1-methyl-1H-pyrazole-3-carboxylic acid

To a solution of 2-chloro-5-(3-formyl-1-methyl-1H-pyrazol-4-yl)-N-(tricyclo[3.3.1.1^{3,7}]dec-1-ylmethyl)-benzamide (Example 31 (a)) (300 mg) in N,N-dimethylformamide (5 mL) was added potassium peroxymonosulfate (670 mg) and the mixture was stirred at room temperature for 6 hours. The mixture was purified by Varian NH₂ cartridge, eluting with methanol and then 5% trifluoroacetic acid in methanol. Further purification by RP-HPLC (acetonitrile-.aqueous trifluoroacetic acid, Symmetry) gave the title compound as a colourless solid (50 mg).

MS: APCI(+ve) 428/430 (M+H⁺).

m.p. 166-175° C. dec.

¹H NMR (400 MHz, d₆-DMSO) δ 8.32 (1H, t), 7.63 (1H, s), 7.57 (1H, dd), 7.52 (1H, d), 7.43 (1H, d), 3.99 (3H, s), 2.93 (2H, d), 1.94 (3H, s), 1.70-1.57 (6H, m), 1.54-1.50 (6H, m).

### EXAMPLE 32

### 4-[4-Chloro-3-[[(tricyclo[3.3.1.1^{3,7}]dec-1-ylmethyl)amino]carbonyl]phenyl]-1-methyl-1H-pyrazole-5-carboxylic acid

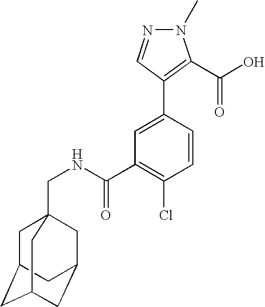

A mixture of [[(tricyclo[3.3.1.1^{3,7}]dec-1-ylmethyl)amino]carbonyl]phenyl]-boronic acid (Example 2 (a)) (350 mg), 4-bromo-1-methyl-1H-pyrazole-5-carboxaldehyde (190 mg), potassium carbonate (300 mg) and tetrakis(triphenylphosphine)palladium(0) (50 mg) in tetrahydrofuran (3 mL)/water (3 mL) was heated at 65° C. with stirring under a nitrogen atmosphere for 6 hours. The products were filtered through diatomaceous earth, washing with methanol (3×10 mL) and the volatile components were removed in vacuo. The residue was purified by chromatography (SiO₂, dichloromethane methanol 99:1 as eluant) and then redissolved in N,N-dimethylformamide (2 mL). Potassium peroxymonosulfate (620 mg) was added, the mixture was stirred at room temperature for 18 hours and the products were purified by Varian NH₂ cartridge, eluting with methanol and then 5% trifluoroacetic acid in methanol. Further purification by recrystallisation from acetonitrile gave the title compound as a colourless solid (20 mg).

MS: APCI(+ve) 428 (M+H⁺).

m.p. 187-190° C. dec.

¹H NMR (300 MHz, d₆-DMSO) δ 8.35 (1H, t), 7.63 (1H, s), 7.51-7.41 (3H, m), 4.07 (3H, s), 2.93 (2H, d), 1.94 (3H, s), 1.72-1.55 (6H, m), 1.52 (6H, s).

### EXAMPLE 33

### N-[3-[4-Chloro-3-[[(tricyclo[3.3.1.1^{3,7}]dec-1-ylmethyl)amino]carbonyl]phenyl]pyrazinyl]-N-methyl-glycine

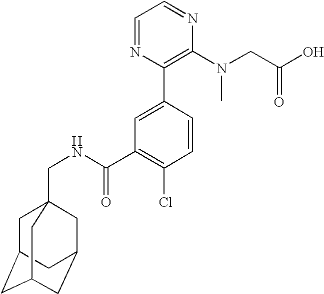

a) N-(3-Chloropyrazinyl)-N-methyl-glycine, 1,1-dimethylethyl ester

A mixture of 2,3-dichloropyrazine (500 mg), N-methyl-glycine, 1,1-dimethylethyl ester hydrochloride (610 mg) and triethylamine (0.94 mL) in acetonitrile (1 mL) were heated in a microwave at 130° C. for 3 hours. The mixture was then concentrated and purified by chromatography (SiO₂, dichloromethane) to give the sub-title compound as an oil (400 mg).

MS: APCI(+ve) 202 (M−C₄H₈+H⁺)

b) N-[3-[4-Chloro-3-[[(tricyclo[3.3.1.1^{3,7}]dec-1-ylmethyl)amino]carbonyl]phenyl]pyrazinyl]-N-methyl-glycine

A mixture of [[(tricyclo[3.3.1.1^{3,7}]dec-1-ylmethyl)amino]carbonyl]phenyl]-boronic acid (Example 2 (a)) (380 mg), N-(3-chloropyrazinyl)-N-methyl-glycine, 1,1-dimethylethyl ester (Example 33 (a)) (400 mg), potassium carbonate (300 mg) and tetrakis(triphenylphosphine)palladium(0) (60 mg) in tetrahydrofuran (3 mL)/water (3 mL) was stirred at room temperature under a nitrogen atmosphere for 16 hours. The products were filtered through diatomaceous earth, washing with methanol (3×10 mL) and the volatile components were removed in vacuo. The residue was partitioned between dichloromethane (25 mL) and water (10 mL), the layers were separated and the organic layer was dried (MgSO₄), filtered and concentrated. Purification by chromatography (SiO₂, isohexane:ethyl acetate 3:1, then isohexane:ethyl acetate 1:1) gave an oil which was dissolved in dichloromethane (5 mL). Trifluoroacetic acid (2 mL) was added, the mixture was stirred at room temperature for 24 hours and then concentrated. Purification by Varian NH₂ resin, eluting with methanol and then 5% trifluoroacetic acid in methanol, and then by trituration with diethyl ether, gave the title compound as a solid (13 mg).

MS: APCI(+ve) 469 (M+H⁺).

m.p. 151-153° C. dec.

¹H NMR (400 MHz, d₆-DMSO) δ 8.38 (1H, t), 8.12 (1H, d), 8.09 (1H, d), 7.77 (1H, dd), 7.71 (1H, d), 7.57 (1H, d), 4.08 (2H, s), 2.95 (2H, d), 2.70 (s, 3H), 1.94 (3H, s), 1.71-1.56 (6H, m), 1.55-1.50 (6H, m).

### EXAMPLE 34

### 1-[3-[4-Chloro-3-[[(tricyclo[3.3.1.1^{3,7}]dec-1-ylmethyl)amino]carbonyl]phenyl]pyrazinyl]-4-piperidinecarboxylic acid

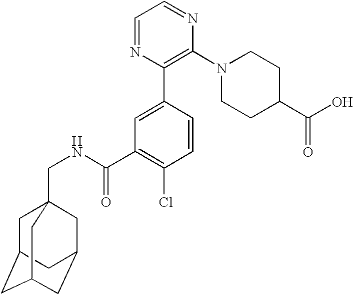

a) 1-[3-[4-Chloro-3-[[(tricyclo[3.3.1.1^{3,7}]dec-1-ylmethyl)amino]carbonyl]phenyl]pyrazinyl]-4-piperidinecarboxylic acid, ethyl ester

A mixture of 2-chloro-5-(3-chloropyrazinyl)-N-(tricyclo[3.3.1.1^{3,7}]dec-1-ylmethyl)-benzamide (Example 5 (a)) (200 mg), ethyl isonipecotate (230 mg) and acetonitrile (1 mL) was heated in a microwave at 140° C. for 6 hours. The products were partitioned between dichloromethane (25 mL) and 2M aqueous hydrochloric acid (10 mL), the layers were separated and the aqueous layer was extracted with dichloromethane (25 mL). The combined organics were washed with saturated aqueous sodium chloride (10 mL), dried (MgSO₄), filtered and concentrated in vacuo. Purification by chromatography (SiO₂, dichloromethane:Pmethanol 99.5:0.5 as eluant) gave the sub-title compound as a film (140 mg).

MS: APCI(+ve) 537 (M+H⁺)

b) 1-[3-[4-Chloro-3-[[(tricyclo[3.3.1.1^{3,7}]dec-1-ylmethyl)amino]carbonyl]phenyl]pyrazinyl]-4-piperidinecarboxylic acid

Prepared according to the method of Example 23 (d) using 1-[3-[4-chloro-3-[[(tricyclo[3.3.1.1^{3,7}]dec-1-ylmethyl)amino]carbonyl]phenyl]pyrazinyl]-4-piperidinecarboxylic acid, ethyl ester (Example 34 (a)) (140 mg). Purification by chromatography (SiO₂, dichloromethane:methanol 98:2 as eluant) gave the title compound as a solid (86 mg).

MS: APCI(+ve) 509 (M+H⁺).

m.p. 136-140° C.

¹H NMR (300 MHz, d₆-DMSO) δ 12.22 (1H, s), 8.39 (1H, t), 8.22 (1H, d), 8.20 (1H, d), 8.00 (1H, dd), 7.95 (1H, d), 7.60 (1H, d), 3.56-3.45 (2H, m), 2.95 (2H, d), 2.83-2.71 (2H, m), 2.44-2.31 (1H, m), 1.99-1.90 (3H, m), 1.85-1.75 (2H, m), 1.72-1.47 (14H, m).

### EXAMPLE 35

### 4′-Chloro-6-fluoro-3′-[[(tricyclo[3.3.1.1^{3,7}]dec-1-ylmethyl)amino]carbonyl]-[1,1′-biphenyl]-2-carboxylic acid

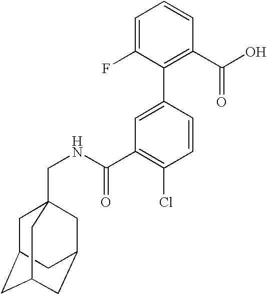

a) 2-Bromo-3-fluoro-benzoic acid, methyl ester

Prepared according to the method of Example 13 (a) using 2-bromo-3-fluoro-benzoic acid (500 mg). Purification by chromatography (SiO₂, dichloromethane as eluant) gave the sub-title compound as a colourless oil (480 mg).

¹H NMR (400 MHz, CDCl₃) δ 7.58 (1H, ddd), 7.34 (1H, ddd), 7.26 (1H, ddd), 3.95 (3H, s).

b) 4′-Chloro-6-fluoro-3′-[[(tricyclo[3.3.1.1^{3,7}]dec-1-ylmethyl)amino]carbonyl]-[1,1′-biphenyl]-2-carboxylic acid, methyl ester

A mixture of [[(tricyclo[3.3.1.1^{3,7}]dec-1-ylmethyl)amino]carbonyl]phenyl]-boronic acid (Example 2 (a)) (500 mg), 2-bromo-3-fluoro-benzoic acid, methyl ester (Example 35 (a)) (340 mg), potassium carbonate (400 mg) and tetrakis(triphenylphosphine)palladium(0) (80 mg) in tetrahydrofuran (3 mL)/water (3 mL) was stirred at 65° C. under a nitrogen atmosphere for 16 hours. The products were filtered through diatomaceous earth, washing with methanol (3×10 mL) and the volatile components were removed in vacuo. The residue was partitioned between dichloromethane (20 mL) and 2M aqueous hydrochloric acid (10 mL), the layers were separated, the aqueous fraction was extracted with dichloromethane (2×20 mL) and the combined organic layers were washed with saturated aqueous sodium chloride (10 mL) before being dried (MgSO₄), filtered and concentrated. Purification by chromatography (SiO₂, dichloromethane) gave the sub-title compound as a colourless solid (250 mg).

MS: APCI(+ve) 456 (M+H⁺).

c) 4′-Chloro-6-fluoro-3′-[[(tricyclo[3.3.1.1^{3,7}]dec-1-ylmethyl)amino]carbonyl]-[1,1′-biphenyl]-2-carboxylic acid

A solution of sodium hydroxide (66 mg) in water (0.5 mL) was added to a solution of 4′-chloro-6-fluoro-3′-[[(tricyclo[3.3.1.1^{3,7}]dec-1-ylmethyl)amino]carbonyl]-[1,1′-biphenyl]-2-carboxylic acid, methyl ester (Example 35 (b)) (250 mg) in tetrahydrofuran (2 mL) in a 10 mL microwave vial and this was heated at 70° C. for 3 hours within a microwave. The reaction mixture was concentrated and then acidified to pH 5 using 2M aqueous hydrochloric acid. Purification by chromatography (SiO₂, dichloromethane, then dichloromethane methanol 98:2 as eluant) gave the title compound as a colourless solid (55 mg).

MS: APCI(−ve) 440 (M−H⁺).

m.p. 108-113° C.

¹H NMR (400 MHz, d₆-DMSO) δ 8.39 (1H, t), 7.68-7.62 (1H, m), 7.59-7.46 (3H, m), 7.37 (1H, d), 7.29 (1H, d), 2.93 (2H, d), 1.93 (3H, s), 1.70-1.55 (6H, m), 1.52 (6H, s).

### EXAMPLE 36

### 4′-Chloro-5-fluoro-3′-[[(tricyclo[3.3.1.1^{3,7}]dec-1-ylmethyl)amino]carbonyl]-[1,1′-biphenyl]-2-carboxylic acid

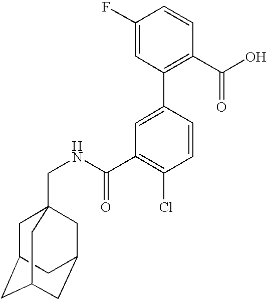

To a stirred solution of 2-bromo-4-fluoro-benzoic acid (500 mg) in dichloromethane (5 mL) at 0° C. under nitrogen was added N,N-dimethylformamide (1 drop) and oxalyl chloride (0.4 mL). The reaction mixture was allowed to warm to room temperature over 2 hours, concentrated in vacuo and then redissolved in dichloromethane (5 mL). Methanol (1 mL) was added dropwise and the mixture was stirred at room temperature for 2 hours before being concentrated in vacuo to give a colourless oil which was dissolved in tetrahydrofuran (3 mL)/water (3 mL). [[(Tricyclo[3.3.1.1^{3,7}]dec-1-ylmethyl)amino]carbonyl]phenyl]-boronic acid (Example 2 (a)) (500 mg), potassium carbonate (400 mg) and tetrakis(triphenylphosphine)palladium(0) (80 mg) were added and the mixture was stirred at 65° C. under a nitrogen atmosphere for 8 hours. The products were filtered through diatomaceous earth, washing with methanol (3×10 mL), and were then concentrated in vacuo. Dichloromethane (20 mL) and 2M aqueous hydrochloric acid (10 mL) were added, the layers were separated and the aqueous fraction was extracted with dichloromethane (2×10 mL). The combined organic layers were washed with saturated aqueous sodium chloride (10 mL), dried (MgSO₄), filtered and concentrated. The residue was dissolved in tetrahydrofuran (3 mL) and a solution of sodium hydroxide (250 mg) in water (3 mL) was added. The mixture was stirred at room temperature for 24 hours, adjusted to pH 5 with 2M aqueous hydrochloric acid and concentrated in vacuo. Purification by chromatography (SiO₂, dichloromethane methanol 98:2 as eluant) gave the title compound as a colourless solid (90 mg).

MS: APCI(−ve) 440 (M−H⁺).

m.p. 120-124° C.

¹H NMR (400 MHz, d₆-DMSO) δ 13.01 (1H, s), 8.36 (1H, t), 7.87 (1H, dd), 7.52 (1H, d), 7.42-7.37 (2H, m), 7.34 (1H, ddd), 7.27 (1H, dd), 2.95 (2H, d), 1.94 (3H, s), 1.71-1.56 (6H, m), 1.55-1.50 (6H, m).

### EXAMPLE 37

### 4′-Chloro-3′-[[(tricyclo[3.3.1.1^{3,7}]dec-1-ylmethyl)amino]carbonyl]-[1,1′-biphenyl]-2-acetic acid

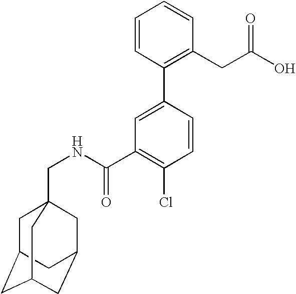

a) 2-Bromo-benzeneacetic acid, methyl ester

Prepared according to the method of Example 13 (a) using 2-bromo-benzeneacetic acid (300 mg), oxalyl chloride (0.4 mL), N,N-dimethylformamide (1 drop), dichloromethane (2 mL) and methanol (2 mL), to give the sub-title compound as a solid (315 mg).

MS: APCI(+ve) 230 (M+H⁺).

b) 4′-Chloro-3′-[[(tricyclo[3.3.1.1^{3,7}]dec-1-ylmethyl)amino]carbonyl]-[1,1′-biphenyl]-2-acetic acid, methyl ester

A mixture of [4-chloro-3-[[(tricyclo[3.3.1.1^{3,7}]dec-1-ylmethyl)amino]carbonyl]phenyl]-boronic acid (Example 2 (a)) (250 mg), 2-bromo-benzeneacetic acid, methyl ester (Example 37 (a)) (198 mg), potassium carbonate (199 mg) and

bis(triphenylphosphine)palladium(II) chloride (50 mg) in tetrahydrofuran (2 mL)/water (1 mL) was heated at 40° C. under a nitrogen atmosphere for 1 hour. The solvent was removed in vacuo and the residue was purified by chromatography (SiO₂, dichloromethane as eluant) to yield the sub-title compound as a solid (100 mg).

MS: APCI(+ve) 452/454 (M+H⁺).

c) 4′-Chloro-3′-[[(tricyclo[3.3.1.1^{3,7}]dec-1-ylmethyl)amino]carbonyl]-[1,1′-biphenyl]-2-acetic acid

Prepared according to the method of Example 10 (b) using 4′-chloro-3′-[[(tricyclo[3.3.1.1^{3,7}]dec-1-ylmethyl)amino]carbonyl]-[1,1′-biphenyl]-2-acetic acid, methyl ester (Example 37 (b)) (100 mg), potassium hydroxide (100 mg), methanol (1 mL) and water (1 mL). Purification (Varian NH₂ cartridge using methanol (100 mL) and then 5% acetic acid in methanol (100 mL) as eluant) afforded the title compound as a solid (19 mg).

MS: APCI(+ve) 438/440 (M+H⁺).

m.p. 200-202° C.

¹H NMR (400 MHz, d₆-DMSO) δ 8.36 (1H, t), 7.55 (1H, d), 7.39-7.32 (4H, m), 7.31 (1H, d), 7.27-7.23 (1H, m), 3.52 (2H, s), 2.94 (2H, d), 1.93 (3H, s), 1.66 (3H, d), 1.59 (3H, d), 1.52 (6H, s).

### EXAMPLE 38

### [[4′-Chloro-3′-[[(tricyclo[3.3.1.1^{3,7}]dec-1-ylmethyl)amino]carbonyl][1,1′-biphenyl]-3-yl]oxy]-acetic acid

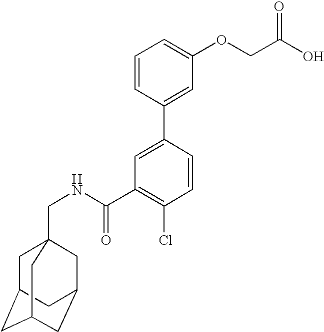

a) 4-Chloro-3′-hydroxy-N-(tricyclo[3.3.1.1^{3,7}]dec-1-ylmethyl)-[1,1′-biphenyl]-3-carboxamide

Prepared according to the method of Example 37 (b) using [4-chloro-3-[[(tricyclo[3.3.1.1^{3,7}]dec-1-ylmethyl)amino]carbonyl]phenyl]-boronic acid (Example 2 (a)) (1 g), 3-bromo-phenol (600 mg), potassium carbonate (800 mg), 6w(triphenylphosphine)palladium(II) chloride (200 mg), tetrahydrofuran (10 mL) and water (10 mL). Purification by chromatography (SiO₂, 1:3 ethyl acetate:isohexane as eluant) gave the sub-title compound as a solid (650 mg).

MS: APCI(+ve) 396/398 (M+H⁺).

m.p. 182-184° C.

¹H NMR (300 MHz, d₆-DMSO) δ 9.59 (1H, s), 8.40 (1H, t), 7.64 (1H, dd), 7.58-7.49 (2H, m), 7.30 (1H, t), 7.09 (1H, d), 7.03 (1H, s), 6.80 (1H, dd), 2.96 (2H, d), 1.95 (3H, s), 1.76-1.46 (12H, m).

b) [[4′-Chloro-3′-[[(tricyclo[3.3.1.1^{3,7}]dec-1-ylmethyl)amino]carbonyl][1,1′-biphenyl]-3-yl]oxy]-acetic acid, ethyl ester

Prepared according to the method of Example 15 (a) using 4-chloro-3′-hydroxy-N-(tricyclo[3.3.1.1^{3,7}]dec-1-ylmethyl)-[1,1′-biphenyl]-3-carboxamide (Example 38 (a)) (250 mg), ethyl chloroacetate (300 mg), potassium carbonate (174 mg) and acetone (4 mL). Purification by chromatography (SiO₂, dichloromethane as eluant) gave the sub-title compound as a solid (180 mg).

MS: APCI(+ve) 482/484 (M+H⁺).

¹H NMR (300 MHz, d₆-DMSO) δ 8.39 (1H, t), 7.72 (1H, dd), 7.65 (1H, d), 7.56 (1H, d), 7.40 (1H, t), 7.29 (1H, d), 7.23 (1H, t), 6.97 (1H, dd), 4.88 (2H, s), 4.18 (2H, q), 2.97 (2H, d), 1.95 (3H, s), 1.68 (3H, d), 1.60 (3H, d), 1.54 (6H, s), 1.21 (3H, t).

c) [[4′-Chloro-3′-[[(tricyclo[3.3.1.1^{3,7}]dec-1-ylmethyl)amino]carbonyl][1,1′-biphenyl]-3-yl]oxy]-acetic acid

Prepared according to the method of Example 15 (b) using [[4′-Chloro-3′-[[(tricyclo[3.3.1.1^{3,7}]dec-1-ylmethyl)amino]carbonyl][1,1′-biphenyl]-3-yl]oxy]-acetic acid, ethyl ester (Example 38 (b)) (180 mg), potassium hydroxide (100 mg), methanol (1 mL) and water (1 mL). Purification by RP-HPLC (acetonitrile:aqueous trifluoroacetic acid, Symmetry) gave the title compound as a solid (22 mg).

MS: APCI(+ve) 454/456 (M+H⁺).

m.p. 99-101° C.

¹H NMR (400 MHz, d₆-DMSO) δ 8.40 (1H, t), 7.72 (1H, dd), 7.65 (1H, d), 7.56 (1H, d), 7.40 (1H, t), 7.27 (1H, d), 7.21 (1H, t), 6.95 (1H, dd), 4.78 (2H, s), 2.97 (2H, d), 1.95 (3H, s), 1.68 (3H, d), 1.60 (3H, d), 1.54 (6H, s).

### EXAMPLE 39

### (2R)-2-[[4′-Chloro-3′-[[(tricyclo[3.3.1.1^{3,7}]dec-1-ylmethyl)amino]carbonyl][1,1′-biphenyl]-2-yl]oxy]-propanoic acid

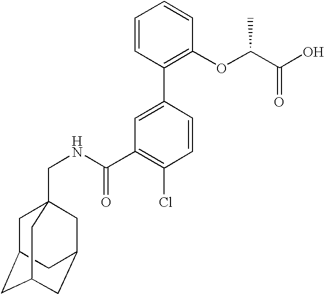

a) (2R)-2-[[4′-chloro-3′-[[(tricyclo[3.3.1.1^{3,7}]dec-1-ylmethyl)amino]carbonyl][1,1′-biphenyl]-2-yl]oxy]-propanoic acid, methyl ester

Prepared according to the method of Example 15 (a) using 4-chloro-2′-hydroxy-N-(tricyclo[3.3.1.1^{3,7}]dec-1-ylmethyl)-[1,1′-biphenyl]-3-carboxamide (Example 14 (a)) (170 mg), methyl (2S)-2-chloropropanoate (212 mg), potassium carbonate (120 mg) and acetone (4 mL). Purification by chromatography (SiO₂, 1:4 ethyl acetate-isohexane) gave the sub-title compound as a solid (140 mg).

MS: APCI(+ve) 482/484 (M+H⁺).

¹H NMR (400 MHz, d₆-DMSO) δ 8.39 (1H, t), 7.67-7.61 (2H, m), 7.52 (1H, d), 7.40-7.28 (2H, m), 7.07 (1H, t), 6.96 (1H, d), 5.10 (1H, q), 3.67 (3H, s), 2.99-2.90 (2H, m), 1.93 (3H, s), 1.67 (3H, d), 1.59 (3H, d), 1.53 (6H, s), 1.45 (3H, d).

b) (2R)-2-[[4′-Chloro-3′-[[(tricyclo[3.3.1.1^{3,7}]dec-1-ylmethyl)amino]carbonyl][1,1′-biphenyl]-2-yl]oxy]-propanoic acid

Prepared according to the method of Example 10 (b) using (2R)-2-[[4′-chloro-3′-[[(tricyclo[3.3.1.1^{3,7}]dec-1-ylmethyl)amino]carbonyl][1,1′-biphenyl]-2-yl]oxy]-propanoic acid, methyl ester (Example 39 (a)) (140 mg), potassium hydroxide (100 mg), methanol (2 mL) and water (1 mL). Purification (Varian NH₂ cartridge using methanol (100 mL) and then 5% acetic acid in methanol (100 mL) as eluant) afforded the title compound as a solid (100 mg).

MS: APCI(+ve) 468/470 (M+H⁺).

m.p. 133-137° C.

¹H NMR (400 MHz, d₆-DMSO) δ 8.43 (1H, t), 7.75 (1H, dd), 7.67 (1H, d), 7.48 (1H, d), 7.31 (1H, d), 7.25 (1H, t), 6.97 (1H, t), 6.90 (1H, d), 4.64 (1H, d), 2.94 (2H, d), 1.92 (3H, s), 1.66 (3H, d), 1.59 (3H, d), 1.52 (6H, s), 1.35 (3H, d).

### EXAMPLE 40

### [[4′-Chloro-3′-[[(tricyclo[3.3.1.1^{3,7}]dec-1-ylmethyl)amino]carbonyl][1,1′-biphenyl]-4-yl]oxy]-acetic acid

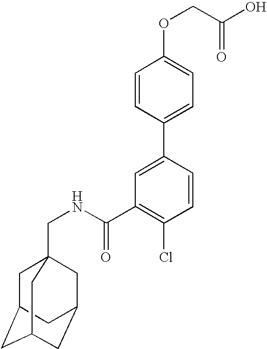

a) 4-chloro-4′-hydroxy-N-(tricyclo[3.3.1.1^{3,7}]dec-1-ylmethyl)-[1,1′-biphenyl]-3-carboxamide

A mixture of [4-chloro-3-[[(tricyclo[3.3.1.1^{3,7}]dec-1-ylmethyl)amino]carbonyl]phenyl]-boronic acid (Example 2 (a)) (1.0 g), 4-bromo-phenol (600 mg), potassium carbonate (800 mg) and bis(triphenylphosphine)palladium(II) chloride (200 mg) in tetrahydrofuran (10 mL)/water (10 mL) was stirred at room temperature under a nitrogen atmosphere for 16 hours. The solvent was removed in vacuo and the residue was purified by chromatography (SiO₂, 1:3 ethyl acetate:isohexane) to yield the sub-title compound as a solid (470 mg).

MS: APCI(+ve) 396/398 (M+H⁺).

b) [[4′-chloro-3′-[[(tricyclo[3.3.1.1^{3,7}]dec-1-ylmethyl)amino]carbonyl][1,1′-biphenyl]-4-yl]oxy]-acetic acid, ethyl ester

Prepared according to the method of Example 15 (a) using 4-chloro-4′-hydroxy-N-(tricyclo[3.3.1.1^{3,7}]dec-1-ylmethyl)-[1,1′-biphenyl]-3-carboxamide (Example 40 (a)) (250 mg), ethyl chloroacetate (300 mg), potassium carbonate (174 mg) and acetone (4 mL). Purification by chromatography (SiO₂, 1:4 ethyl acetate:isohexane as eluant) gave the sub-title compound as a solid (190 mg).

MS: APCI(+ve) 482/484 (M+H⁺). c) [[4′-Chloro-3′-[[(tricyclo[3.3.1.1^{3,7}]dec-1-ylmethyl)amino]carbonyl][1,1′-biphenyl]-4-yl]oxy]-acetic acid

Prepared according to the method of Example 10 (b) using [[4′-chloro-3′-[[(tricyclo[3.3.1.1^{3,7}]dec-1-ylmethyl)amino]carbonyl][1,1′-biphenyl]-4-yl]oxy]-acetic acid, ethyl ester (Example 40 (b)) (190 mg), potassium hydroxide (100 mg), methanol (2 mL) and water (1 mL). Purification by RP-HPLC (acetonitrile:aqueous ammonium acetate, Symmetry) afforded the title compound as a solid (74 mg).

MS: APCI(+ve) 454/456 (M+H⁺).

m.p. 136-140° C.

¹H NMR (400 MHz, d₆-DMSO) δ 8.38 (1H, t), 7.64 (1H, dd), 7.60-7.55 (3H, m), 7.50 (1H, d), 6.92 (2H, d), 4.34 (2H, s), 2.96 (2H, d), 1.95 (3H, s), 1.67 (3H, d), 1.60 (3H, d), 1.54 (6H, s).

### EXAMPLE 41

### (2S)-2-[[4′-Chloro-3′-[[(tricyclo[3.3.1.1^{3,7}]dec-1-ylmethyl)amino]carbonyl][1,1′-biphenyl]-3-yl]oxy]-propanoic acid

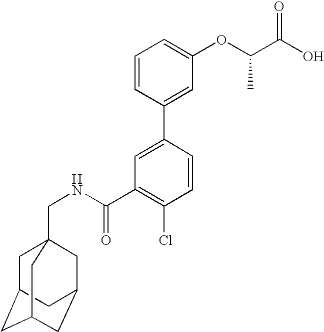

a) (2S)-2-[[4′-chloro-3′-[[(tricyclo[3.3.1.1^{3,7}]dec-1-ylmethyl)amino]carbonyl][1,1′-biphenyl]-3-yl]oxy]-propanoic acid, methyl ester

Prepared according to the method of Example 15 (a) using 4-chloro-3′-hydroxy-N-(tricyclo[3.3.1.1^{3,7}]dec-1-ylmethyl)-[1,1′-biphenyl]-3-carboxamide (Example 38 (a)) (250 mg), methyl (2R)-2-chloropropanoate (311 mg), potassium carbonate (174 mg) and acetone (4 mL). Purification by chromatography (SiO₂, 1:4 ethyl acetate:isohexane) gave the sub-title compound as a solid (200 mg).

MS: APCI(+ve) 482/484 (M+H⁺).

¹H NMR (400 MHz, d₆-DMSO) δ 8.39 (1H, t), 7.70 (1H, dd), 7.63 (1H, d), 7.56 (1H, d), 7.39 (1H, t), 7.28 (1H, d), 7.20 (1H, s), 6.92 (1H, dd), 5.16 (1H, q), 3.68 (3H, s), 2.97 (2H, d), 1.95 (3H, s), 1.73-1.48 (15H, m).

b) (2S)-2-[[4′-Chloro-3′-[[(tricyclo[3.3.1.1^{3,7}]dec-1-ylmethyl)amino]carbonyl][1,1′-biphenyl]-3-yl]oxy]-propanoic acid

Prepared according to the method of Example 10 (b) using (2S)-2-[[4′-chloro-3′-[[(tricyclo[3.3.1.1^{3,7}]dec-1-ylmethyl)amino]carbonyl][1,1′-biphenyl]-3-yl]oxy]-propanoic acid, methyl ester (Example 41 (a)) (200 mg), potassium hydroxide (100 mg), methanol (2 mL) and water (1 mL) to afford the title compound as a solid (175 mg).

MS: APCI(+ve) 468/470 (M+H⁺).

m.p. 115-120° C.

¹H NMR (400 MHz, d₆-DMSO) δ 8.39 (1H, t), 7.69 (1H, dd), 7.62 (1H, d), 7.54 (1H, d), 7.37 (1H, t), 7.24 (1H, d), 7.16 (1H, s), 6.89 (1H, dd), 4.93 (1H, q), 2.96 (2H, d), 1.93 (3H, d), 1.73-1.43 (15H, m).

### EXAMPLE 42

### 4,4′-Dichloro-3′-[[(tricyclo[3.3.1.1^{3,7}]dec-1-ylmethyl)amino]carbonyl]-[1,1′-biphenyl]-2-carboxylic acid

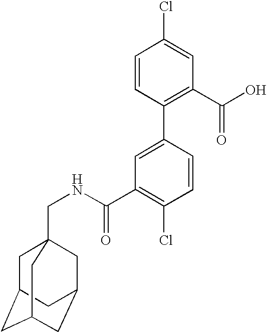

a) 4,4′-Dichloro-3′-[[(tricyclo[3.3.1.1^{3,7}]dec-1-ylmethyl)amino]carbonyl]-[1,1′-biphenyl]-2-carboxylic acid, methyl ester

Prepared according to the method of Example 40 (a) using [4-chloro-3-[[(tricyclo[3.3.1.1^{3,7}]dec-1-ylmethyl)amino]carbonyl]phenyl]-boronic acid (Example 2 (a)) (250 mg), methyl 2-bromo-5-chlorobenzoate (215 mg), potassium carbonate (200 mg), bis(triphenylphosphine)palladium(II) chloride (50 mg), tetrahydrofuran (2 mL) and water (2 mL). Purification by chromatography (SiO₂, 1:4 ethyl acetate:isohexane) gave the sub-title compound as a solid (200 mg).

MS: APCI(+ve) 472/474 (M+H⁺).

¹H NMR (400 MHz, d₆-DMSO) δ 8.40 (1H, t), 7.83 (1H, d), 7.74 (1H, dd), 7.58-7.47 (2H, m), 7.36 (1H, dd), 7.30 (1H, d), 3.65 (3H, d), 2.95 (2H, d), 1.94 (3H, s), 1.67 (3H, d), 1.60 (3H, d), 1.52 (6H, s).

b) 4,4′-Dichloro-3′-[[(tricyclo[3.3.1.1^{3,7}]dec-1-ylmethyl)amino]carbonyl]-[1,1′-biphenyl]-2-carboxylic acid

Prepared according to the method of Example 10 (b) using 4,4′-dichloro-3′-[[(tricyclo[3.3.1.1^{3,7}]dec-1-ylmethyl)amino]carbonyl]-[1,1′-biphenyl]-2-carboxylic acid, methyl ester (Example 42 (a)) (200 mg), potassium hydroxide (100 mg), methanol (2 mL) and water (1 mL). Purification (Varian NH₂ cartridge using methanol (100 mL) and then 5% acetic acid in methanol (100 mL) as eluant) afforded the title compound as a solid (125 mg). MS: APCI(−ve) 456 (M−H⁺).

m.p. 207-210° C.

¹H NMR (400 MHz, d₆-DMSO) δ 8.38 (1H, t), 7.78 (1H, d), 7.68 (1H, dd), 7.52 (1H, d), 7.44 (1H, d), 7.40-7.33 (2H, m), 2.94 (2H, d), 1.94 (3H, s), 1.67 (3H, d), 1.59 (3H, d), 1.52 (6H, s).

### EXAMPLE 43

### (2S)-2-[[4′-Chloro-3′-[[(tricyclo[3.3.1.1^{3,7}]dec-1-ylmethyl)amino]carbonyl][1,1′-biphenyl]-4-yl]oxy]-propanoic acid

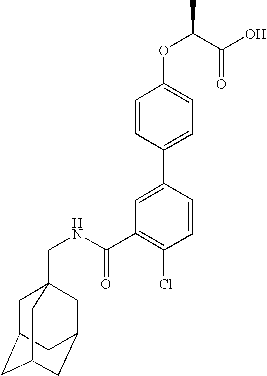

a) (2S)-2-[[4′-chloro-3′-[[(tricyclo[3.3.1.1^{3,7}]dec-1-ylmethyl)amino]carbonyl][1,1′-biphenyl]-4-yl]oxy]-propanoic acid, methyl ester

Prepared according to the method of Example 15 (a) using 4-chloro-4′-hydroxy-N-(tricyclo[3.3.1.1^{3,7}]dec-1-ylmethyl)-[1,1′-biphenyl]-3-carboxamide (Example 40 (a)) (210 mg), methyl (2R)-2-chloropropanoate (262 mg), potassium carbonate (150 mg) and acetone (4 mL). Purification by chromatography (SiO₂, 1:2 ethyl acetate:isohexane) gave the sub-title compound as a solid (140 mg).

MS: APCI(+ve) 482/484 (M+H⁺).

b) (2S)-2-[[4′-Chloro-3′-[[(tricyclo[3.3.1.1^{3,7}]dec-1-ylmethyl)amino]carbonyl][1,1′-biphenyl]-4-yl]oxy]-propanoic acid

Prepared according to the method of Example 10 (b) using (2S)-2-[[4′-chloro-3′-[[(tricyclo[3.3.1.1^{3,7}]dec-1-ylmethyl)amino]carbonyl][1,1′-biphenyl]-4-yl]oxy]-propanoic acid, methyl ester (Example 43 (a)) (140 mg), potassium hydroxide (100 mg), methanol (2 mL) and water (1 mL). Purification by RP-HPLC (acetonitrile:aqueous ammonium acetate, Symmetry) afforded the title compound as a solid (50 mg).

MS: APCI(+ve) 468/470 (M+H⁺).

m.p. 145-150° C.

¹H NMR (400 MHz, d₆-DMSO) δ 8.38 (1H, t), 7.63 (1H, dd), 7.60-7.53 (3H, m), 7.50 (1H, d), 6.91 (2H, d), 4.59 (1H, q), 2.96 (2H, d), 1.94 (3H, s), 1.67 (3H, d), 1.60 (3H, d), 1.54 (6H, s), 1.43 (3H, d).

### EXAMPLE 44

### 3-Chloro-6-[4-chloro-3-[[(tricyclo[3.3.1.1^{3,7}]dec-1-ylmethyl)amino]carbonyl]phenyl]-2-pyridinecarboxylic acid

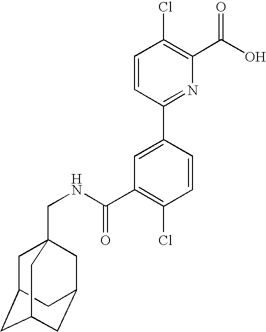

a) 3-Chloro-6-[4-chloro-3-[[(tricyclo[3.3.1.1^{3,7}]dec-1-ylmethyl)amino]carbonyl]phenyl]-2-pyridinecarboxylic acid, methyl ester

Prepared according to the method of Example 40 (a) using [4-chloro-3-[[(tricyclo[3.3.1.1^{3,7}]dec-1-ylmethyl)amino]carbonyl]phenyl]-boronic acid (Example 2 (a)) (200 mg), methyl 3,6-dichloropyridine-2-carboxylate (123 mg), potassium carbonate (166 mg), bis(triphenylphosphine)palladium(II) chloride (42 mg), tetrahydrofuran (2 mL) and water (2 mL). Purification by chromatography (SiO₂, 1:4 ethyl acetate:isohexane) gave the sub-title compound as a solid (130 mg).

MS: APCI(+ve) 473/475 (M+H⁺).

b) 3-Chloro-6-[4-chloro-3-[[(tricyclo[3.3.1.1^{3,7}]dec-1-ylmethyl)amino]carbonyl]phenyl]-2-pyridinecarboxylic acid

Prepared according to the method of Example 10 (b) using 3-chloro-6-[4-chloro-3-[[(tricyclo[3.3.1.1^{3,7}]dec-1-ylmethyl)amino]carbonyl]phenyl]-2-pyridinecarboxylic acid, methyl ester (Example 44 (a)) (130 mg), potassium hydroxide (100 mg), methanol (2 mL) and water (1 mL). Purification by chromatography (SiO₂, 1:9 methanol:dichloromethane) gave the title compound as a solid (100 mg).

MS: APCI(+ve) 459/461 (M+H⁺).

m.p. 154-159° C.

¹H NMR (400 MHz, d₆-DMSO) δ 8.45 (1H, t), 8.24-8.09 (4H, m), 7.64 (1H, d), 2.98 (2H, d), 1.95 (3H, s), 1.68 (3H, d), 1.61 (3H, d), 1.55 (6H, s).

### EXAMPLE 45

### 3-[4-Chloro-3-[[(tricyclo[3.3.1.1^{3,7}]dec-1-ylmethyl)amino]carbonyl]phenyl]-4-pyridinecarboxylic acid

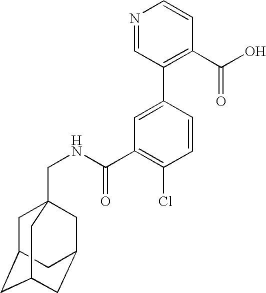

a) 3-[4-Chloro-3-[[(tricyclo [3.3.1.1^{3,7}]dec-1-ylmethyl)amino]carbonyl]phenyl]-4-pyridinecarboxylic acid, methyl ester

Prepared according to the method of Example 37 (b) using [4-chloro-3-[[(tricyclo[3.3.1.1^{3,7}]dec-1-ylmethyl)amino]carbonyl]phenyl]-boronic acid (Example 2 (a)) (250 mg), 3-iodo-4-pyridinecarboxylic acid, methyl ester (190 mg), potassium carbonate (200 mg), bis(triphenylphosphine)palladium(II) chloride (51 mg), tetrahydrofuran (2 mL) and water (2 mL). Purification by chromatography (SiO₂, 1:3 ethyl acetate:isohexane as eluant) gave the sub-title compound as a solid (80 mg).

MS: APCI(+ve) 439/441 (M+H⁺).

¹H NMR (300 MHz, CDCl₃) δ 8.75 (1H, d), 8.67 (1H, d), 7.72-7.66 (2H, m), 7.48 (1H, d), 7.33 (1H, dd), 6.38-6.27 (1H, m), 3.76 (3H, s), 3.19 (2H, d), 2.02 (3H, s), 1.79-1.54 (12H, m).

b) 3-[4-Chloro-3-[[(tricyclo[3.3.1.1^{3,7}]dec-1-ylmethyl)amino]carbonyl]phenyl]-4-pyridinecarboxylic acid

Prepared according to the method of Example 10 (b) using 3-[4-chloro-3-[[(tricyclo[3.3.1.1^{3,7}]dec-1-ylmethyl)amino]carbonyl]phenyl]-4-pyridinecarboxylic acid, methyl ester (Example 45 (a)) (80 mg), potassium hydroxide (100 mg), methanol (2 mL) and water (1 mL). Purification by chromatography (SiO₂, 1:9 methanol:dichloromethane as eluant) gave the title compound as a solid (30 mg).

MS: APCI(−ve) 423/425 (M−H⁺).

m.p. 170-240° C. dec.

¹H NMR (400 MHz, d₆-DMSO) δ 8.64-8.55 (2H, m), 8.38 (1H, t), 7.56-7.44 (4H, m), 2.94 (2H, d), 1.93 (3H, s), 1.66 (3H, d), 1.59 (3H, d), 1.53 (6H, s).

### EXAMPLE 46

### [[2-[4-Chloro-3-[[(tricyclo[3.3.1.1^{3,7}]dec-1-ylmethyl)amino]carbonyl]phenyl]-3-pyridinyl]oxy]-acetic acid

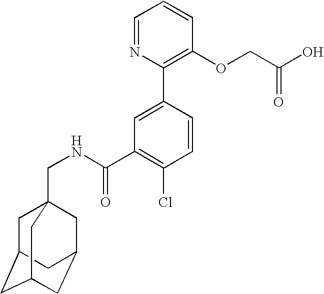

a) 2-Chloro-5-(3-hydroxy-2-pyridinyl)-N-(tricyclo[3.3.1.1^{3,7}]dec-1-ylmethyl)-benzamide

Prepared according to the method of Example 26 (a) using [4-chloro-3-[[(tricyclo[3.3.1.1^{3,7}]dec-1-ylmethyl)amino]carbonyl]phenyl]-boronic acid (Example 2 (a)) (2.0 g), 3-hydroxy-2-bromopyridine (1.0 g), sodium carbonate (1.27 g), tetrakis(triphenylphosphine)palladium(0) (693 mg), toluene (20 mL), ethanol (20 mL) and water (20 mL). Purification by chromatography (SiO₂, 1:1 ethyl acetate:isohexane as eluant) gave the sub-title compound as a solid (900 mg).

MS: APCI(+ve) 397/399 (M+H⁺).

¹H NMR (400 MHz, d₆-DMSO) δ 10.38 (1H, s), 8.38 (1H, t), 8.17 (1H, dd), 8.14-8.09 (2H, m), 7.53 (1H, d), 7.36 (1H, dd), 7.24 (1H, dd), 2.96 (2H, d), 1.95 (3H, s), 1.68 (3H, d), 1.60 (3H, d), 1.54 (6H, s).

b) [[2-[4-Chloro-3-[[(tricyclo[3.3.1.1^{3,7}]dec-1-ylmethyl)amino]carbonyl]phenyl]-3-pyridinyl]oxy]-acetic acid, ethyl ester

Prepared according to the method of Example 15 (a) using 2-chloro-5-(3-hydroxy-2-pyridinyl)-N-(tricyclo[3.3.1.1^{3,7}]dec-1-ylmethyl)-benzamide (Example 46 (a)) (250 mg), ethyl chloroacetate (300 mg), potassium carbonate (174 mg) and acetone (4 mL). Purification by chromatography (SiO₂, 1:2 ethyl acetate:isohexane as eluant) gave the sub-title compound as a solid (150 mg).

MS: APCI(+ve) 483/485 (M+H⁺).

¹H NMR (400 MHz, d₆-DMSO) δ 8.38 (1H, t), 8.31 (1H, dd), 8.07 (1H, dd), 8.03 (1H, d), 7.57-7.52 (2H, m), 7.38 (1H, dd), 4.95 (2H, s), 4.18 (2H, q), 2.95 (2H, d), 1.94 (3H, s), 1.67 (3H, d), 1.60 (3H, d), 1.53 (6H, s), 1.21 (3H, t).

c) [[2-[4-Chloro-3-[[(tricyclo[3.3.1.1^{3,7}]dec-1-ylmethyl)amino]carbonyl]phenyl]-3-pyridinyl]oxy]-acetic acid

Prepared according to the method of Example 10 (b) using [[2-[4-chloro-3-[[(tricyclo[3.3.1.1^{3,7}]dec-1-ylmethyl)amino]carbonyl]phenyl]-3-pyridinyl]oxy]-acetic acid, ethyl ester (Example 46 (b)) (150 mg), potassium hydroxide (100 mg), methanol (2 mL) and water (1 mL). Purification (Varian NH₂ cartridge using dichloromethane (100 mL) and then 5% acetic acid in dichloromethane (100 mL) as eluant) afforded the title compound as a solid (139 mg).

MS: APCI(−ve) 453/455 (M−H⁺).

m.p. 133-137° C.

¹H NMR (300 MHz, d₆-DMSO) δ 8.38 (1H, t), 8.28 (1H, dd), 8.07 (1H, dd), 8.02 (1H, d), 7.54 (1H, d), 7.49 (1H, dd), 7.36 (1H, dd), 4.80 (2H, s), 2.95 (2H, d), 1.94 (3H, s), 1.67 (3H, d), 1.59 (3H, d), 1.53 (6H, s).

### EXAMPLE 47

### N-[2-[4-Chloro-3-[[(tricyclo[3.3.1.1^{3,7}]dec-1-ylmethyl)amino]carbonyl]phenyl]-3-pyridinyl]-glycine

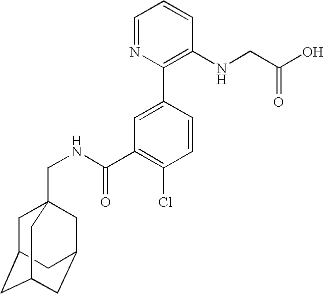

a) 2-Chloro-5-(3-nitro-2-pyridinyl)-N-(tricyclo[3.3.1.1^{3,7}]dec-1-ylmethyl)-benzamide

Prepared according to the method of Example 37 (b) using [4-chloro-3-[[(tricyclo[3.3.1.1^{3,7}]dec-1-ylmethyl)amino]carbonyl]phenyl]-boronic acid (Example 2 (a)) (1.0 g), 2-chloro-3-nitropyridine (460 mg), potassium carbonate (800 mg), bis(triphenylphosphine)palladium(II) chloride (100 mg), tetrahydrofuran (10 mL) and water (10 mL). Purification by chromatography (SiO₂, 1:9 ethyl acetate:isohexane as eluant) gave the sub-title compound as a solid (600 mg).

MS: APCI(+ve) 426/428 (M+H⁺).

¹H NMR (400 MHz, d₆-DMSO) δ 8.95 (1H, dd), 8.54-8.44 (2H, m), 7.76-7.71 (1H, m), 7.64-7.55 (3H, m), 2.96 (2H, t), 1.94 (3H, s), 1.67 (3H, d), 1.60 (3H, d), 1.53 (6H, s).

b) 5-(3-Amino-2-pyridinyl)-2-chloro-N-(tricyclo[3.3.1.1^{3,7}]dec-1-ylmethyl)-benzamide

Iron powder (600 mg) was added portionwise to a stirred mixture of 2-chloro-5-(3-nitro-2-pyridinyl)-N-(tricyclo[3.3.1.1^{3,7}]dec-1-ylmethyl)-benzamide (Example 47 (a)) (600 mg), ammonium chloride (600 mg), ethanol (5 mL) and water (5 mL). The mixture was then heated at 50° C. for 24 hours then allowed to cool and filtered through diatomaceous earth, washing with dichloromethane (200 mL). The filtrate and washings were evaporated and the combined residues were partitioned between dichloromethane (100 mL) and water (100 mL). The layers were separated and the organics were dried, filtered and evaporated to afford the sub-title compound as a solid (450 mg).

MS: APCI(+ve) 396/398 (M+H⁺).

¹H NMR (400 MHz, d₆-DMSO) δ 8.37-8.26 (1H, m), 7.92 (1H, dd), 7.78-7.67 (2H, m), 7.59-7.52 (1H, m), 7.15 (1H, dd), 7.09 (1H, dd), 5.26 (2H, s), 2.96 (2H, d), 1.95 (3H, s), 1.74-1.46 (12H, m).

c) N-[2-[4-Chloro-3-[[(tricyclo[3.3.1.1^{3,7}]dec-1-ylmethyl)amino]carbonyl]phenyl]-3-pyridinyl]-glycine

Ethyl glyoxalate (50% solution in toluene) (200 mg) was added to a stirred mixture of 5-(3-amino-2-pyridinyl)-2-chloro-N-(tricyclo[3.3.1.1^{3,7}]dec-1-ylmethyl)-benzamide (Example 47 (b)) (400 mg) and activated 3 Å molecular sieves (500 mg) in dichloromethane (5 mL). The mixture was stirred at room temperature under nitrogen for 3 hours, sodium triacetoxyborohydride (642 mg) was added, stirring was continued for a further 16 hours and the mixture was then concentrated in vacuo. Purification by chromatography (SiO₂, ethyl acetate, then 1:9 methanol:ethyl acetate as eluant) afforded the title compound (18 mg).

MS: APCI(−ve) 452/454 (M−H⁺).

m.p. 190-194° C.

¹H NMR (400 MHz, d₆-DMSO) δ 8.47 (1H, t), 7.88 (1H, d), 7.70 (1H, dd), 7.64 (1H, d), 7.58 (1H, d), 7.15 (1H, d), 6.90 (1H, d), 5.49 (1H, s), 3.48-3.16 (2H, m), 2.95 (2H, d), 1.93 (3H, s), 1.66 (3H, d), 1.59 (3H, d), 1.52 (6H, s).

### EXAMPLE 48

### 4′-Chloro-4,5-difluoro-3′-[[(tricyclo[3.3.1.1^{3,7}]dec-1-ylmethyl)amino]carbonyl]-[1,1′-biphenyl]-2-carboxylic acid

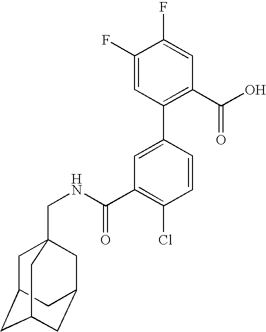

a) 2-Bromo-4,5-difluoro-benzoic acid, methyl ester

Prepared according to the method of Example 13 (a) using 2-bromo-4,5-difluorobenzoic acid (237 mg), oxalyl chloride (0.1 mL), N,N-dimethylformamide (1 drop), dichloromethane (2 mL) and methanol (2 mL) to give the sub-title compound as a solid (250 mg).

¹H NMR (300 MHz, CDCl₃) δ 7.74 (1H, dd), 7.51 (1H, dd), 3.93 (3H, s).

b) 4′-Chloro-4,5-difluoro-3′-[[(tricyclo[3.3.1.1^{3,7}]dec-1-ylmethyl)amino]carbonyl]-[1,1′-biphenyl]-2-carboxylic acid, methyl ester

Prepared according to the method of Example 40 (a) using [4-chloro-3-[[(tricyclo[3.3.1.1^{3,7}]dec-1-ylmethyl)amino]carbonyl]phenyl]-boronic acid (Example 2 (a)) (200 mg), 2-bromo-4,5-difluoro-benzoic acid, methyl ester (Example 48 (a)) (175 mg), potassium carbonate (170 mg), bis(triphenylphosphine)palladium(II) chloride (20 mg), tetrahydrofuran (1 mL) and water (1 mL). Purification by chromatography (SiO₂, 1:4 ethyl acetate:isohexane as eluant) gave the sub-title compound as a solid (60 mg).

MS: APCI(+ve) 474 (M+H⁺).

c) 4′-Chloro-4,5-difluoro-3′-[[(tricyclo[3.3.1.1^{3,7}]dec-1-ylmethyl)amino]carbonyl]-[1,1′-biphenyl]-2-carboxylic acid

A solution of potassium hydroxide (50 mg) in water (0.5 mL) was added to a solution of 4′-chloro-4,5-difluoro-3′-[[(tricyclo[3.3.1.1^{3,7}]dec-1-ylmethyl)amino]carbonyl]-[1,1′-biphenyl]-2-carboxylic acid, methyl ester (Example 48 (b)) (60 mg), in methanol (0.5 mL) and tetrahydrofuran (0.5 mL). The mixture was stirred at room temperature for 2 hours then concentrated. The residue was dissolved in water (5 mL) and the solution was acidified to pH 2 with 2 M aqueous hydrochloric acid. The resulting solid was collected by filtration and washed with water to afford the title compound (50 mg).

MS: APCI(−ve) 458 (M−H⁺).

m.p. 121-125° C.

¹H NMR (400 MHz, d₆-DMSO) δ 8.35 (1H, t), 7.84 (1H, dd), 7.56-.48 (2H, m), 7.41-7.36 (2H, m), 2.94 (2H, d), 1.94 (3H, s), 1.67 (3H, d), 1.59 (3H, d), 1.52 (6H, s).

### EXAMPLE 49

### 4′-Chloro-3′-[[(2-tricyclo[3.3.1.1^{3,7}]dec-1-ylethyl)amino]carbonyl]-[1,1′-biphenyl]-2-carboxylic acid

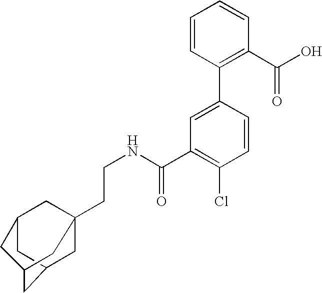

a) 2-Chloro-5-iodo-benzoic acid, 1,1-dimethylethyl ester

N,N-Dimethylformamide (1 drop) and oxalyl chloride (4.8 mL) were added to a stirred solution of 2-chloro-5-iodobenzoic acid (5 g) in dichloromethane (20 mL) at 0° C. The reaction was allowed to warm to room temperature, stirred under nitrogen for 2 hours, and then evaporated to dryness. The residue was dissolved in tetrahydrofuran (20 mL) and cooled to 0° C. Potassium tert-butoxide (22 mL, 1 M solution in tetrahydrofuran) was added over 10 minutes. The reaction was allowed to warm to room temperature and stirred under nitrogen for 2 hours then poured into saturated aqueous sodium bicarbonate (50 mL). The layers were separated and the aqueous was extracted with diethyl ether (50 mL). The combined organics were dried, filtered and evaporated to afford the sub-title compound as an oil (5.7 g).

¹H NMR (400 MHz, d₆-DMSO) δ 7.99 (1H, d), 7.87 (1H, dd), 7.34 (1H, d), 1.54 (9H, s).

b) 2-Chloro-5-(4,4,5,5-tetramethyl-1,3,2-dioxaborolan-2-yl)-benzoic acid, 1,1-dimethylethyl ester

A mixture of 2-Chloro-5-iodo-benzoic acid, 1,1-dimethylethyl ester (Example 49 (a)) (5 g), bis(pinacolato)diboron (6 g), [1,1′-bis(diphenylphosphino)ferrocene]dichloropalladium(II)dichoromethane (600 mg) and potassium acetate (6.5 g) in N,N-dimethylformamide (50 mL) was heated to 90° C. under nitrogen for 90 minutes. The mixture was allowed to cool then diluted with 2:1 ethyl acetate:diethyl ether (250 mL) and filtered through diatomaceous earth. The filtrate was washed with water (250 mL) and brine (100 mL) then evaporated. Purification (SiO₂, 1:1 diethyl ether:isohexane as eluant) afforded the sub-title compound as a solid (5.5 g).

MS: APCI(+ve) 282 (M−C₄H₈+H⁺).

¹H NMR (300 MHz, d₆-DMSO) δ 7.88 (1H, d), 7.76 (1H, dd), 7.56 (1H, d), 1.55 (9H, s), 1.32 (12H, s).

c) 4′-Chloro-[1,1′-biphenyl]-2,3′-dicarboxylic acid, 3′-(1,1-dimethylethyl) 2-methyl ester

Prepared according to the method of Example 40 (a) using 2-chloro-5-(4,4,5,5-tetramethyl-1,3,2-dioxaborolan-2-yl)-benzoic acid, 1,1-dimethylethyl ester (Example 49 (b)) (3.5 g), methyl-2-bromobenzoate (2.23 g), potassium carbonate (2.87 g), bis(triphenylphosphine)palladium(II) chloride (365 mg), tetrahydrofuran (20 mL) and water (20 mL). Purification by chromatography (SiO₂, 98:2 isohexane:ethyl acetate as eluant) gave the sub-title compound as a solid (2.15 g).

¹H NMR (300 MHz, d₆-DMSO) δ 7.82 (1H, dd), 7.67 (1H, td), 7.61-7.5.2 (3H, m), 7.49-7.43 (2H, m), 3.64 (3H, s), 1.55 (9H, s).

d) 4′-Chloro-[1,1′-biphenyl]-2,3′-dicarboxylic acid, 2-methyl ester

Trifluoroacetic acid (3.3 mL) was added to a stirred solution of 4′-chloro-[1,1′-biphenyl]-2,3′-dicarboxylic acid, 3′-(1,1-dimethylethyl) 2-methyl ester (Example 49 (c)) (2.15 g) in dichloromethane (10 mL) and the mixture was stirred at room temperature under nitrogen for 90 minutes. The mixture was then evaporated to afford the sub-title compound as a solid (1.7 g).

¹H NMR (400 MHz, d₆-DMSO) δ 7.82 (1H, dd), 7.69-7.64 (2H, m), 7.59 (1H, d), 7.55 (1H, td), 7.49-7.44 (2H, m), 3.63 (3H, s).

e) 4′-Chloro-3′-[[(2-tricyclo[3.3.1.1^{3,7}]dec-1-ylethyl)amino]carbonyl]-[1,1′-biphenyl]-2-carboxylic acid, methyl ester N,N-Dimethylformamide (1 drop) and oxalyl chloride (0.16 mL) were added to a stirred solution of 4′-chloro-[1,1′-biphenyl]-2,3′-dicarboxylic acid, 2-methyl ester (Example 49 (d)) (170 mg) in dichloromethane (2 mL) at 0° C. The reaction was allowed to warm to room temperature, stirred under nitrogen for 2 hours, then evaporated to dryness. The residue was dissolved in dichloromethane (2 mL) and cooled to 0° C. [2-(1-adamantyl)ethyl]amine hydrochloride (153 mg) was added followed by triethylamine (0.24 mL). The reaction was allowed to warm to room temperature and stirred under nitrogen for 2 hours then poured into saturated aqueous sodium bicarbonate (20 mL). The aqueous was extracted with dichloromethane (3×20 mL). The combined organic fractions were dried (MgSO₄), filtered and evaporated. Purification by chromatography (SiO₂, 1:4 ethyl acetate:isohexane as eluant) afforded the sub-title compound as a solid (260 mg).

MS: APCI(+ve) 452/454 (M+H⁺).

¹H NMR (400 MHz, d₆-DMSO) δ 8.36 (1H, t), 7.79 (1H, dd), 7.66 (1H, td), 7.56-7.49 (2H, m), 7.46 (1H, dd), 7.32 (1H, dd), 7.29 (1H, d), 3.63 (3H, s), 3.27-3.21 (2H, m), 1.93 (3H, s), 1.73-1.56 (6H, m), 1.51 (6H, s), 1.35-1.28 (2H, m).

f) 4′-Chloro-3′-[[(2-tricyclo[3.3.1.1^{3,7}]dec-1-ylethyl)amino]carbonyl]-[1,1′-biphenyl]-2-carboxylic acid

Prepared according to the method of Example 48 (c), using 4′-chloro-3′-[[(2-tricyclo[3.3.1.1^{3,7}]dec-1-ylethyl)amino]carbonyl]-[1,1′-biphenyl]-2-carboxylic acid, methyl ester (Example 49 (e)) (260 mg), potassium hydroxide (100 mg), water (1 mL), methanol (1 mL) and tetrahydrofuran (1 mL), to afford the title compound as a solid (140 mg).

MS: APCI(−ve) 436 (M−H⁺).

m.p. 114-117° C.

¹H NMR (400 MHz, d₆-DMSO) δ 8.36 (1H, t), 7.77 (1H, dd), 7.59 (1H, td), 7.52-7.46 (2H, m), 7.42-7.34 (2H, m), 7.32 (1H, d), 3.23 (2H, dt), 1.93 (3H, s), 1.67 (3H, d), 1.61 (3H, d), 1.51 (6H, s), 1.34-1.28 (2H, m).

### EXAMPLE 50

### 3-[4-Chloro-3-[[(2-tricyclo[3.3.1.1^{3,7}]dec-1-ylethyl)amino]carbonyl]phenyl]-2-pyridinecarboxylic acid

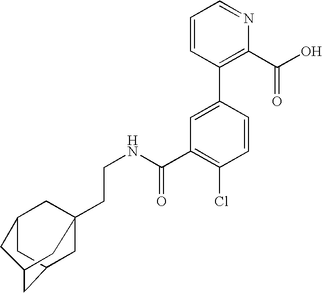

a) 3-Iodo-2-pyridinecarboxylic acid, methyl ester

Butyllithium (32 mL, 2.5 M in hexanes) was added dropwise over 10 minutes to a solution of 2,2,6,6-tetramethylpiperidine (10.2 mL) in tetrahydrofuran (100 mL) at −78° C. under nitrogen. The mixture was stirred at −78° C. for 15 minutes and then picolinic acid (2.4 g) was added portionwise over 10 minutes. After a further 10 minutes at −78° C. the mixture was allowed to warm to 0° C. and stirred under nitrogen for 30 minutes. The reaction mixture was then added dropwise over 15 minutes to a solution of iodine (15 g) in tetrahydrofuran (100 mL) at 0° C. This was then allowed to warm to room temperature and stirred for 1 hour before water (20 mL) was added. The mixture was evaporated to dryness to leave a black oil. Dichloromethane (50 mL) was added and the mixture was cooled to 0° C. N,N-Dimethylformamide (1 drop) and oxalyl chloride (4 mL) were added. The reaction was allowed to warm to room temperature and stirred under nitrogen for 2 hours, then evaporated to dryness. The residue was dissolved in dichloromethane (20 mL) and then methanol (20 mL) was added. The mixture was then stirred for 10 minutes before being evaporated to afford the sub-title compound as an oil (1.0 g) which was used in the next step without purification.

MS: APCI(+ve) 264 (M+H⁺).

b) 3-[4-Chloro-3-[(1,1-dimethylethoxy)carbonyl]phenyl]-2-pyridinecarboxylic acid, methyl ester

2-Chloro-5-(4,4,5,5-tetramethyl-1,3,2-dioxaborolan-2-yl)-benzoic acid, 1,1-dimethylethyl ester (Example 49 (b)) (500 mg), 3-iodo-2-pyridinecarboxylic acid, methyl ester (Example 50 (a)) (400 mg) and tetrahydrofuran (2 mL) were placed in a 10 mL microwave vial. A solution of potassium carbonate (400 mg) in water (1 mL) was added followed by bis(triphenylphosphine)palladium(II) chloride (50 mg), and the mixture was heated to 130° C. in a microwave for 3 hours then concentrated. The residue was partitioned between dichloromethane (20 mL) and water (20 mL). The layers were separated and the aqueous was extracted with dichoromethane (2×20 mL). The combined organics were dried, filtered and evaporated. Purification by chromatography (SiO₂, 1:4 ethyl acetate:isohexane as eluant) gave the sub-title compound as a solid (240 mg).

MS: APCI(+ve) 348/450 (M+H⁺).

¹H NMR (400 MHz, d₆-DMSO) δ 8.68 (1H, dd), 8.02 (1H, dd), 7.71-7.63 (3H, m), 7.54 (1H, dd), 3.71 (3H, s), 1.56 (9H, s).

c) 3-(3-Carboxy-4-chlorophenyl)-2-pyridinecarboxylic acid, 2-methyl ester

Prepared according to the method of Example 49 (d), using 3-[4-chloro-3-[(1,1-dimethylethoxy)carbonyl]phenyl]-2-pyridinecarboxylic acid, methyl ester (Example 50 (b)) (240 mg), trifluoroacetic acid (1 mL) and dichloromethane (3 mL) to afford the sub-title compound as an oil (200 mg).

MS: APCI(+ve) 292/294 (M+H⁺).

¹H NMR (300 MHz, CDCl₃) δ 8.97 (1H, dd), 8.14 (1H, dd), 8.00 (1H, d), 7.91 (1H, dd), 7.64 (1H, d), 7.48 (1H, dd), 3.88 (3H, d).

d) 3-[4-Chloro-3-[[(2-tricyclo[3.3.1.1^{3,7}]dec-1-ylethyl)amino]carbonyl]phenyl]-2-pyridinecarboxylic acid, methyl ester

Prepared according to the method of Example 49 (e), using 3-(3-carboxy-4-chlorophenyl)-2-pyridinecarboxylic acid, 2-methyl ester (Example 50 (c)) (170 mg), N,N-dimethylformamide (1 drop), oxalyl chloride (0.16 mL), [2-(1-adamantyl)ethyl]amine hydrochloride (150 mg) and dichloromethane (4 mL). Purification (SiO₂, 2% methanol in dichloromethane) afforded the sub-title compound as a solid (200 mg).

MS: APCI(+ve) 453/455 (M+H⁺).

¹H NMR (400 MHz, CDCl₃) δ 8.71 (1H, dd), 7.74 (1H, dd), 7.66 (1H, d), 7.51 (1H, dd), 7.45 (1H, d), 7.31 (1H, dd), 6.17 (1H, s), 3.85 (3H, s), 3.53-3.45 (2H, m), 1.97 (3H, s), 1.72 (3H, d), 1.64 (3H, d), 1.56 (6H, s), 1.45-1.38 (2H, m),

e) 3-[4-Chloro-3-[[(2-tricyclo[3.3.1.1^{3,7}]dec-1-ylethyl)amino]carbonyl]phenyl]-2-pyridinecarboxylic acid

Prepared according to the method of Example 48 (c), using 3-[4-chloro-3-[[(2-tricyclo[3.3.1.1^{3,7}]dec-1-ylethyl)amino]carbonyl]phenyl]-2-pyridinecarboxylic acid, methyl ester (Example 50 (d)) (200 mg), potassium hydroxide (100 mg), water (1 mL), methanol (1 mL) and tetrahydrofuran (1 mL). Purification (Varian NH₂ cartridge using dichloromethane (100 mL) and then 5% acetic acid in dichloromethane (100 mL) as eluant) afforded the title compound as a solid (85 mg).

MS: APCI(+ve) 439/441 (M+H⁺).

m.p. 108-111° C.

¹H NMR (300 MHz, d₆-DMSO) δ 8.62 (1H, dd), 8.39 (1H, t), 7.92 (1H, dd), 7.65-7.52 (2H, m), 7.50-7.40 (2H, m), 3.30-3.14 (2H, m), 1.92 (3H, s), 1.75-1.45 (12H, m), 1.36-1.27 (2H, m).

### EXAMPLE 51

### 4′-Chloro-4-fluoro-3′-[[(tricyclo[3.3.1.1^{3,7}]dec-1-ylmethyl)amino]carbonyl]-[1,1′-biphenyl]-2-carboxylic acid

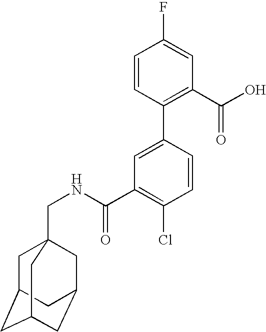

a) 2-Bromo-5-fluoro-benzoic acid, methyl ester

Prepared according to the method of Example 13 (a) using 2-bromo-5-fluoro-benzoic acid (160 mg), oxalyl chloride (0.2 mL), N,N-dimethylformamide (1 drop), dichloromethane (2 mL) and methanol (2 mL) to give the sub-title compound as a solid (170 mg).

¹H NMR (400 MHz, CDCl₃) δ 7.63 (1H, dd), 7.53 (1H, dd), 7.07 (1H, ddd), 3.94 (3H, s).

b) 4′-Chloro-4-fluoro-3′-[[(tricyclo[3.3.1.1^{3,7}]dec-1-ylmethyl)amino]carbonyl]-[1,1′-biphenyl]-2-carboxylic acid, methyl ester

[4-Chloro-3-[[(tricyclo[3.3.1.1^{3,7}]dec-1-ylmethyl)amino]carbonyl]phenyl]-boronic acid (Example 2 (a)) (200 mg), 2-bromo-5-fluoro-benzoic acid, methyl ester (Example 51 (a)) (170 mg), tetrahydrofuran (2 mL), a solution of potassium carbonate (166 mg) in water (1 mL) and bis(triphenylphosphine)palladium(II) chloride (20 mg) were placed in a 10 mL microwave vial. The mixture was heated to 70° C. for 1 hour in a microwave then evaporated. The residue was partitioned between dichloromethane (20 mL) and water (20 mL). The layers were separated and the aqueous was extracted with dichloromethane (2×20 mL). The combined organics were dried (MgSO₄,), filtered and evaporated. Purification by chromatography (SiO₂, 1:4 ethyl acetate:isohexane as eluant) gave the sub-title compound as an oil (79 mg).

MS: APCI(+ve) 456/458 (M+H⁺).

c) 4′-Chloro-4-fluoro-3′-[[(tricyclo[3.3.1.1^{3,7}]dec-1-ylmethyl)amino]carbonyl]-[1,1′-biphenyl]-2-carboxylic acid

Prepared according to the method of Example 48 (c), using 4′-chloro-4-fluoro-3′-[[(tricyclo[3.3.1.1^{3,7}′]dec-1-ylmethyl)amino]carbonyl]-[1,1′-biphenyl]-2-carboxylic acid, methyl ester (Example 51 (b)) (79 mg), potassium hydroxide (50 mg), water (0.5 mL), methanol (0.5 mL) and tetrahydrofuran (0.5 mL) to afford the title compound as a solid (65 mg).

MS: APCI(−ve) 440 (M−H⁺).

m.p. 126-128° C.

¹H NMR (400 MHz, d₆-DMSO) δ 8.37 (1H, t), 7.56 (1H, d), 7.50 (1H, d), 7.48-7.43 (2H, m), 7.39-7.32 (2H, m), 2.94 (2H, d), 1.94 (3H, s), 1.67 (3H, d), 1.59 (3H, d), 1.52 (6H, s).

### EXAMPLE 52

### 2-[5-Chloro-4-[[(tricyclo[3.3.1.1^{3,7}]dec-1-ylmethyl)amino]carbonyl]-2-pyridinyl]-benzoic acid

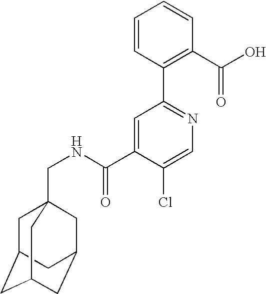

(a) 2-[5-Chloro-4-[[(tricyclo[3.3.1.1^{3,7}]dec-1-ylmethyl)amino]carbonyl]-2-pyridinyl]-benzoic acid, ethyl ester

To a mixture of 2,5-dichloro-N-(tricyclo[3.3.1.1^{3,7}]dec-1-ylmethyl)-4-pyridinecarboxamide (Prepared as described in WO 01/94338) (250 mg), 2-(4,4,5,5-tetramethyl-1,3,2-dioxaborolan-2-yl)-benzoic acid, ethyl ester (204 mg) and potassium carbonate (100 mg) in tetrahydrofuran (3 mL) and water (0.5 mL) was added tetrakis(triphenylphosphine)palladium(0) (20 mg). The mixture was heated at 80° C. under a nitrogen atmosphere for 17 hours and subsequently cooled to room temperature and water (5 mL) added. The resulting solid was collected by filtration and washed with water (10 mL). Drying of the solid yielded the sub-title compound as a solid (250 mg).

MS: APCI(−ve) 451/453 (M−H⁺).

(b) 2-[5-Chloro-4-[[(tricyclo[3.3.1.1^{3,7}]dec-1-ylmethyl)amino]carbonyl]-2-pyridinyl]-benzoic acid

To a solution of 2-[5-chloro-4-[[(tricyclo[3.3.1.1^{3,7}]dec-1-ylmethyl)amino]carbonyl]-2-pyridinyl]-benzoic acid, ethyl ester (Example 52 (a)) (250 mg) in methanol (4 mL) was added aqueous sodium hydroxide solution (40%, 2 mL). The mixture was stirred at room temperature for 18 hours, acidified with 2M aqueous hydrochloric acid and the resulting solid removed by filtration. Recrystallisation (acetonitrile) yielded the title compound as a colourless solid (180 mg).

MS: APCI(+ve) 425/427 (M+H⁺).

m.p. 134-138° C.

¹H NMR (400 MHz, d₆-DMSO) δ 12.82 (1H, s), 8.69 (1H, d), 8.65 (1H, t), 7.74 (1H, d), 7.66-7.61 (3H, m), 7.57 (1H, m), 2.97 (2H, d), 1.95 (3H, s), 1.72-1.56 (6H, m), 1.51-1.53 (6H, m).

### EXAMPLE 53

### 2-[4-Chloro-3-[[(tricyclo[3.3.1.1^{3,7}]dec-1-ylmethyl)amino]carbonyl]phenyl]-4-methyl-3-pyridinecarboxylic acid

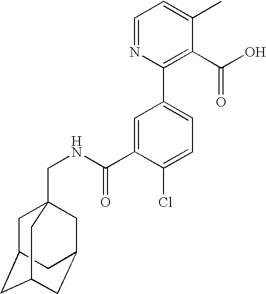

a) 4-Methyl-3-pyridinecarboxylic acid, butyl ester

4-Methyl-3-pyridinecarboxylic acid hydrochloride (1.5 g) and n-butanol (6 mL) were heated in a microwave at 180° C. for 90 minutes. Saturated aqueous sodium hydrogen carbonate was added and the mixture was extracted three times with dichloromethane. The combined organics were washed with water, brine, dried over magnesium sulphate, filtered and the solvent removed to afford the sub-title compound (1.0 g).

MS: APCI(+ve) 194 (M+H⁺).

b) 4-Methyl-3-pyridinecarboxylic acid 1-oxide, butyl ester

To a stirred solution of 4-methyl-3-pyridinecarboxylic acid, butyl ester (Example 53 (a)) (1.0 g) in dichloromethane (5 mL) was added 36-40% peracetic acid (1 mL). After stirring for 12 hours, saturated aqueous sodium hydrogen carbonate was added and the mixture was extracted three times with dichloromethane. The combined organics were washed with water, brine, dried over magnesium sulphate, filtered and the solvent removed to afford the sub-title compound as an oil (1.0 g).

MS: APCI(+ve) 210 (M+H⁺).

c) 2-Chloro-4-methyl-3-pyridinecarboxylic acid, butyl ester

To 4-methyl-3-pyridinecarboxylic acid 1-oxide, butyl ester (Example 53 (b)) (1.0 g) was added phosphorus oxychloride (2 mL) which was then heated at 80° C. for 5 hours. The volatile components were removed under reduced pressure, ice was then added and the mixture was stirred for 2 hours. The mixture was extracted three times with dichloromethane and the combined organics were washed with saturated aqueous sodium hydrogen carbonate, brine, dried over magnesium sulphate, filtered and the solvent removed to afford the sub-title compound as a brown oil (500 mg).

MS: APCI(+ve) 228 (M+H⁺).

d) 2-[4-Chloro-3-[[(tricyclo[3.3.1.1^{3,7}]dec-1-ylmethyl)amino]carbonyl]phenyl]-4-methyl-3-pyridinecarboxylic acid

2-Chloro-4-methyl-3-pyridinecarboxylic acid, butyl ester (Example 53 (c)) (250 mg), [4-chloro-3-[[(tricyclo[3.3.1.1^{3,7}]dec-1-ylmethyl)amino]carbonyl]phenyl]-boronic acid (Example 2 (a)) (347 mg), sodium carbonate (318 mg), tetrakis(triphenylphosphine)palladium(0) (20 mg), tetrahydrofuran (2 mL), and water (1 mL) were heated in a microwave for 90 minutes at 120° C. 48% sodium hydroxide solution (200 μl) was added and the mixture was heated in a microwave at 90° C. for 60 minutes. The products were acidified with 2M aqueous hydrochloric acid, extracted three times with ethyl acetate, the combined organics were washed with brine, dried over magnesium sulphate, filtered and the solvent removed in vacuo. Purification by chromatography (SiO₂, dichloromethane:methanol:acetic acid 96.5:3:0.5 as eluant) and then by trituration with diethyl ether afforded the title compound as a solid (19 mg).

MS: APCI(+ve) 439 (M+H⁺).

m.p. 177-180° C.

¹H NMR (400 MHz, d₆-DMSO) δ 13.45 (1H, s), 8.62 (1H, d), 8.27 (1H, t), 7.41-7.38 (1H, m), 7.33 (1H, d), 7.26-7.22 (2H, m), 4.39 (3H, s), 2.90 (2H, d), 1.93 (3H, s), 1.70-1.47 (12H, m).

### EXAMPLE 54

### 6-[4-Chloro-3-[[(tricyclo[3.3.1.1^{3,7}]dec-1-ylmethyl)amino]carbonyl]phenyl]-2-[(2-hydroxyethyl)methylamino]-3-pyridinecarboxylic acid

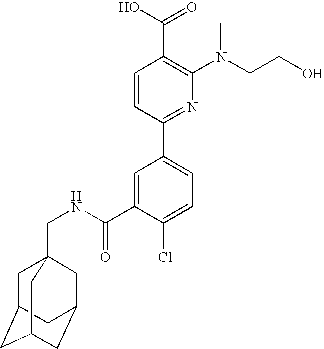

a) 6-Chloro-2-[(2-hydroxyethyl)methylamino]-3-pyridinecarboxylic acid

2,6-Dichloro-3-pyridinecarboxylic acid (500 mg) and 2-(methylamino)-ethanol (586 mg) were stirred in acetonitrile (2 mL) for 12 hours and then heated in a microwave at 70° C. for 60 minutes. The solvent was removed under vacuum to afford the sub-title compound (600 mg).

MS: APCI(−ve) 229 (M−H⁺).

b) 6-[4-Chloro-3-[[(tricyclo[3.3.1.1^{3,7}]dec-1-ylmethyl)amino]carbonyl]phenyl]-2-[(2-hydroxyethyl)methylamino]-3-pyridinecarboxylic acid

6-Chloro-2-[(2-hydroxyethyl)methylamino]-3-pyridinecarboxylic acid (Example 54 (a)) (240 mg), [4-chloro-3-[[(tricyclo[3.3.1.1^{3,7}]dec-1-ylmethyl)amino]carbonyl]phenyl]-boronic acid (Example 2(a)) (200 mg), sodium carbonate (122 mg), tetrakis(triphenylphosphine)palladium (0) (20 mg), tetrahydrofuran (1 mL), and water (1 mL) were heated in a microwave for 30 minutes at 120° C. The reaction was acidified with 2M aqueous hydrochloric acid and extracted three times with ethyl acetate. The combined organics were washed with brine, dried (MgSO₄), filtered and concentrated in vacuo. Purification by RP-HPLC (acetonitrile:aqueous trifluroacetic acid, Symmetry) and then by trituration with diethyl ether afforded the title compound as a solid (72 mg).

MS: APCI(+ve) 498 (M+H⁺).

m.p. 121-124° C.

¹H NMR (300 MHz, d₆-DMSO) δ 8.44 (1H, t), 8.14-8.06 (2H, m), 7.96 (1H, d), 7.59 (1H, d), 7.40-7.32 (1H, m), 3.70-3.58 (4H, m), 3.02 (3H, s), 2.97 (2H, d), 1.95 (3H, s), 1.73-1.52 (12H, m).

### EXAMPLE 55

### 3-[4-Methyl-3-[[(tricyclo[3.3.1.1^{3,7}]dec-1-ylmethyl)amino]carbonyl]phenyl]-2-pyridinecarboxylic acid

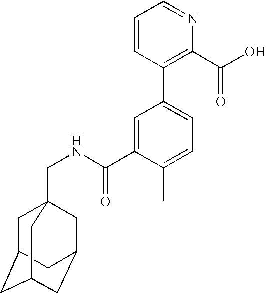

a) 5-Iodo-2-methyl-N-(tricyclo[3.3.1.1^{3,7}]dec-1-ylmethyl)-benzamide

5-Iodo-2-methyl-benzoic acid (3.0 g) was stirred in dichloromethane (40 mL) under nitrogen. Oxalyl chloride (5 mL) was added followed by N,N-dimethylformamide (1 drop). After 2 hours the volatiles were removed under vacuum and the residue was redissolved in dichloromethane (40 mL). Tricyclo[3.3.1.1^{3,7}]decane-1-methanamine (2.23 mL) and triethylamine (3.18 mL) were added and the mixture was stirred under nitrogen for 2 hours. 2M aqueous hydrochloric acid was added, the layers were separated and the aqueous fraction was extracted twice with dichloromethane. The combined organics were washed with water, brine, dried (MgSO₄), filtered and concentrated in vacuo. Purification by trituration with diethyl ether afforded the sub-title compound (4.7 g).

MS: APCI(+ve) 410 (M+H⁺).

b) 2-Methyl-5-(4,4,5,5-tetramethyl-1,3,2-dioxaborolan-2-yl)-N-(tricyclo[3.3.1.1^{3,7}]dec-1-ylmethyl)-benzamide

5-Iodo-2-methyl-N-(tricyclo[3.3.1.1^{3,7}]dec-1-ylmethyl)-benzamide (Example 55 (a)) (500 mg), 4,4,4′,4′,5,5,5′,5′-octamethyl-2,2′-bi-1,3,2-dioxaborolane (496 mg), potassium acetate (539 mg), tetrakis(triphenylphosphine)palladium(0) (20 mg), and N,N-dimethylformamide (2 mL) were heated at 90° C. for 60 minutes in a microwave. Ethyl acetate and water were added, the layers were separated and the aqueous phase was extracted twice with ethyl acetate. The combined organics were washed with water, brine, dried (MgSO₄), filtered and concentrated in vacuo. Purification by chromatography (SiO₂, dichloromethane as eluant) afforded the sub-title compound (292 mg).

MS: APCI(+ve) 410 (M+H⁺).

c) 3-[4-Methyl-3-[[(tricyclo[3.3.1.1^{3,7}]dec-1-ylmethyl)amino]carbonyl]phenyl]-2-pyridinecarboxylic acid

2-Methyl-5-(4,4,5,5-tetramethyl-1,3,2-dioxaborolan-2-yl)-N-(tricyclo[3.3.1.1^{3,7}]dec-1-ylmethyl)-benzamide (Example 55 (b)) (146 mg), 3-iodo-2-pyridinecarboxylic acid, methyl ester (Example 50 (a)) (113 mg), sodium carbonate (113 mg), tetrakis(triphenylphosphine)palladium(0) (10 mg), tetrahydrofuran (1 mL) and water (1 mL) were heated in a microwave at 120° C. for 60 minutes. 48% sodium hydroxide solution (300 μL) was added and the mixture was heated for 30 minutes at 100° C. in a microwave. The reaction mixture was acidified with 2M aqueous hydrochloric acid, the layers were separated and the aqueous fraction was extracted three times with ethyl acetate. The combined organic layers were washed with water and the solvent was removed under vacuum. Purification by chromatography (SiO₂, dichloromethane:methanol:acetic acid 95:4:1 as eluant) and then by trituration with diethyl ether afforded the title compound (63 mg).

MS: APCI(+ve) 405 (M+H⁺).

m.p. 181-183° C.

¹H NMR (400 MHz, d₆-DMSO) δ 13.35 (1H, s), 8.59 (1H, dd), 8.19 (1H, t), 7.93 (1H, dd), 7.61 (1H, dd), 7.40-7.37 (2H, m), 7.33 (1H, d), 2.95 (2H, d), 2.39 (3H, s), 1.94 (3H, s), 1.72-1.55 (6H, m), 1.52-1.49 (6H, m).

### EXAMPLE 56

### 4-[4-Chloro-3-[[(tricyclo[3.3.1.1^{3,7}]dec-1-ylmethyl)amino]carbonyl]phenyl]-1,3-dimethyl-1H-pyrazole-5-carboxylic acid

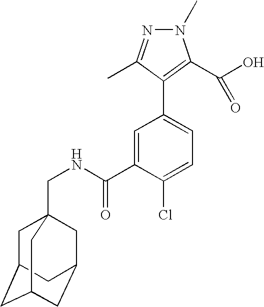

Prepared according to the method of Example 55 (c) using [4-chloro-3-[[(tricyclo[3.3.1.1^{3,7}]dec-1-ylmethyl)amino]carbonyl]phenyl]-boronic acid (Example 2(a)) (139 mg) and 4-bromo-1,3-dimethyl-1H-pyrazole-5-carboxylic acid, methyl ester (140 mg). Purification by chromatography (SiO₂, dichloromethane:methanol:acetic acid 98.5:1:0.5 as eluant) gave the title compound (25 mg).

MS: APCI(+ve) 442 (M+H⁺).

m.p. 207° C.

¹H NMR (300 MHz, d₆-DMSO) δ 13.30 (1H, s), 8.35 (1H, t), 7.48 (1H, d), 7.33 (1H, dd), 7.26 (1H, d), 4.02 (3H, s), 2.93 (2H, d), 2.09 (3H, s), 1.97-1.90 (3H, m), 1.71-1.49 (12H, m).

### EXAMPLE 57

### 2-[4-Methyl-3-[[(tricyclo[3.3.1.1^{3,7}]dec-1-ylmethyl)amino]carbonyl]phenyl]-3-pyridinecarboxylic acid

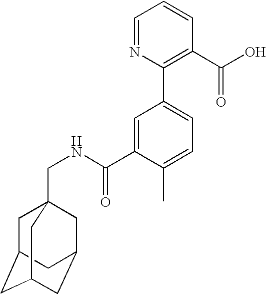

2-Methyl-5-(4,4,5,5-tetramethyl-1,3,2-dioxaborolan-2-yl)-N-(tricyclo[3.3.1.1^{3,7}]dec-1-ylmethyl)-benzamide (Example 55 (b)) (90 mg), 2-chloro-3-pyridinecarboxylic acid, ethyl ester (53 mg), sodium carbonate (70 mg), tetrakis(triphenylphosphine)palladium(0) (15 mg), tetrahydrofuran (1 mL) and water (1 mL) were heated in a microwave at 120° C. for 100 minutes. 48% sodium hydroxide solution (300 μL) was added and the mixture was heated for 60 minutes at 110° C. in a microwave. The reaction was acidified with 2M aqueous hydrochloric acid, the layers were separated and the aqueous phase was extracted three times with ethyl acetate. The combined organic layers were washed with water and the solvent was removed under vacuum. Purification by RP-HPLC, (acetonitrile:aqueous trifluroacetic acid, Symmetry) gave the title compound (60 mg).

MS: APCI(+ve) 405 (M+H⁺).

¹H NMR (300 MHz, d₆-DMSO) δ 8.76 (1H, dd), 8.20 (1H, t), 8.11 (1H, dd), 7.64-7.45 (3H, m), 7.31 (1H, d), 2.95 (2H, d), 2.40 (3H, s), 1.94 (3H, s), 1.63 (6H, q), 1.53-1.48 (6H, m).

### EXAMPLE 58

### 3-[4-Chloro-3-[[(tricyclo[3.3.1.1^{3,7}]dec-1-ylmethyl)amino]carbonyl]phenyl]-2-pyridineacetic acid, monosodium salt

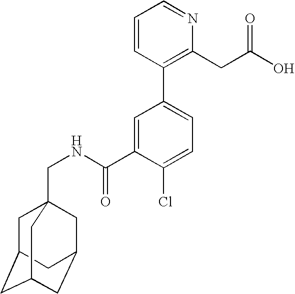

[4-Chloro-3-[[(tricyclo[3.3.1.1^{3,7}]dec-1-ylmethyl)amino]carbonyl]phenyl]-boronic acid (Example 2(a)) (142 mg), 3-bromo-2-pyridineacetic acid ethyl ester (Prepared according to the method of Synthesis, 1997,949-952) (100 mg), sodium carbonate (130 mg), tetrakis(triphenylphosphine)palladium(0) (10 mg), tetrahydrofuran (2 mL) and water (1 mL) were heated in a microwave at 120° C. for 30 minutes. 48% sodium hydroxide solution (200 μL) was added to the reaction which was stirred for 12 hours before being filtered. The solid was washed with water (3 mL) and then acetonitrile (3 mL) before being dried in a vacuum oven to afford the title compound as a solid (55 mg).

MS: APCI(−ve) 437 (M−H⁺).

m.p. 186-187° C. dec.

¹H NMR (300 MHz, d₆-DMSO) δ 8.61 (1H, t), 8.44 (1H, d), 7.72 (1H, dd), 7.61-7.55 (2H, m), 7.49 (1H, d), 7.26-7.19 (1H, m), 3.29 (2H, s), 2.94 (2H, d), 1.93 (3H, s), 1.70-1.50 (12H, m).

### EXAMPLE 59

### 1-[3-[4-Chloro-3-[[(tricyclo[3.3.1.1^{3,7}]dec-1-ylmethyl)amino]carbonyl]phenyl]-2-pyridinyl]-4-piperidinecarboxylic acid

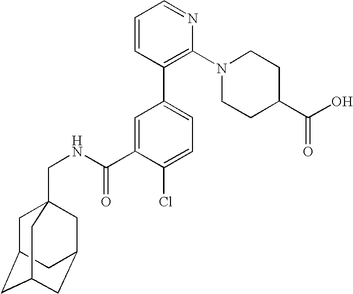

a) 1-(3-Bromo-2-pyridinyl)-4-piperidinecarboxylic acid, ethyl ester

A mixture of 2,3-dibromopyridine (250 mg), ethyl isonipecotate (250 mg) and triethylamine (0.25 mL) in acetonitrile (0.5 mL) was heated at 130° C. in a microwave for 3 hours. The mixture was then concentrated to dryness in vacuo and partitioned between dichloromethane and water. The organics were collected, dried over magnesium sulphate, filtered and concentrated to dryness to give the sub-title compound as a brown oil (325 mg).

MS: APCI(+ve) 313/315 (M+H⁺).

b) 1-[3-[4-Chloro-3-[[(tricyclo[3.3.1.1^{3,7}]dec-1-ylmethyl)amino]carbonyl]phenyl]-2-pyridinyl]-4-piperidinecarboxylic acid, ethyl ester

A mixture of [4-chloro-3-[[(tricyclo[3.3.1.1^{3,7}]dec-1-ylmethyl)amino]carbonyl]phenyl]-boronic acid (Example 2 (a)) (300 mg), 1-(3-bromo-2-pyridinyl)-4-piperidinecarboxylic acid, ethyl ester (Example 59 (a)) (320 mg), potassium carbonate (240 mg) and bis(triphenylphosphine)palladium(II) chloride (60 mg) in toluene (10 mL)/ethanol (1 mL)/water (1 mL) was heated at 50° C. under a nitrogen atmosphere for 3 hours. The mixture was concentrated in vacuo, passed through a plug of silica, eluting with ethyl acetate, and purified further by chromatography (SiO₂, isohexane:ethyl acetate 7:3 as eluant) to give the sub-title compound as a foam (160 mg).

MS: APCI(+ve) 536/538 (M+H⁺).

c) 1-[3-[4-Chloro-3-[[(tricyclo[3.3.1.1^{3,7}]dec-1-ylmethyl)amino]carbonyl]phenyl]-2-pyridinyl]-4-piperidinecarboxylic acid

To a solution of 1-[3-[4-chloro-3-[[(tricyclo[3.3.1.1^{3,7}]dec-1-ylmethyl)amino]carbonyl]phenyl]-2-pyridinyl]-4-piperidinecarboxylic acid, ethyl ester (Example 59 (b)) (160 mg) in tetrahydrofuran (2 mL) and ethanol (1 mL) was added a solution of potassium hydroxide (220 mg) in water (1 mL). The resulting solution was stirred at room temperature for 16 hours. The reaction mixture was then acidified by the addition of acetic acid and purified (Varian NH₂ cartridge using acetonitrile and then 50% acetic acid in acetonitrile as eluant) to give the title compound (90 mg).

MS: APCI(+ve) 508/510 (M+H⁺).

m.p. 110-115° C.

¹H NMR (400 MHz, d₆-DMSO) δ 8.37 (1H, t), 8.19 (1H, dd), 7.72 (1H, dd), 7.60 (1H, d), 7.57 (1H, dd), 7.54 (1H, d), 6.99 (1H, dd), 3.37 (2H, d), 2.95 (2H, d), 2.64 (2H, t), 1.80-1.38 (20H, m).

### EXAMPLE 60

### 1-[3-[4-Chloro-3-[[(tricyclo[3.3.1.1^{3,7}]dec-1-ylmethyl)amino]carbonyl]phenyl]-2-pyridinyl]-L-proline

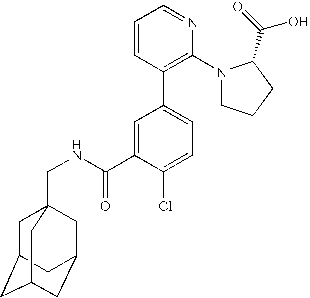

a) 1-(3-Bromo-2-pyridinyl)-L-proline, 1,1-dimethylethyl ester

Prepared according to the method of Example 59 (a) using 2,3-dibromopyridine (250 mg) and L-proline 1,1-dimethylethyl ester (250 mg) to give the sub-title compound as an oil (325 mg).

¹H NMR (400 MHz, CDCl₃) δ 8.03 (1H, dd), 7.66 (1H, dd), 6.51 (1H, dd), 4.65 (1H, dd), 3.87-4.01 (2H, m), 2.18-2.33 (1H, m), 1.90-2.13 (3H, m), 1.40 (9H, s).

b) 1-[3-[4-chloro-3-[[(tricyclo[3.3.1.1^{3,7}]dec-1-ylmethyl)amino]carbonyl]phenyl]-2-pyridinyl]-L-proline, 1,1-dimethylethyl ester

Prepared according to the method of Example 59 (b) using [4-chloro-3-[[(tricyclo[3.3.1.1^{3,7}]dec-1-ylmethyl)amino]carbonyl]phenyl]-boronic acid (Example 2 (a)) (300 mg) and 1-(3-bromo-2-pyridinyl)-L-proline, 1,1-dimethylethyl ester (Example 60 (a)) (320 mg) to give the sub-title compound as a foam (100 mg)

MS: APCI(+ve) 550/552 (M+H⁺)

c) 1-[3-[4-Chloro-3-[[(tricyclo[3.3.1.1^{3,7}]dec-1-ylmethyl)amino]carbonyl]phenyl]-2-pyridinyl]-L-proline

To a solution of 1-[3-[4-chloro-3-[[(tricyclo[3.3.1.1^{3,7}]dec-1-ylmethyl)amino]carbonyl]phenyl]-2-pyridinyl]-L-proline, 1,1-dimethylethyl ester (Example 60 (b)) (200 mg) in dichloromethane (5 mL) was treated with trifluoroacetic acid (1 mL) and heated at reflux for 1 hour. The mixture was neutralised with 7N methanolic ammonia, concentrated to dryness in vacuo and the residue purified (Varian NH₂ cartridge using acetonitrile and then 20% acetic acid in acetonitrile as eluant) to give the title compound as a pale yellow solid (110 mg).

MS: APCI(+ve) 494 (M+H⁺).

m.p. 121-125° C.

¹H NMR (400 MHz, d₆-DMSO) δ 8.52 (1H, s), 8.06 (1H, dd), 7.51 (1H, d), 7.40-7.47 (3H, m), 6.76 (1H, dd), 4.34-4.43 (1H, m), 2.88-3.03 (3H, m), 2.71-2.78 (1H, m), 2.10-2.19 (1H, m), 1.93 (3H, s), 1.67-1.81 (3H, m), 1.66 (3H, d), 1.59 (3H, d), 1.53 (6H, s).

### EXAMPLE 61

### 1-[3-[4-Chloro-3-[[(tricyclo[3.3.1.1^{3,7}]dec-1-ylmethyl)amino]carbonyl]phenyl]-2-pyridinyl]-3-piperidinecarboxylic acid

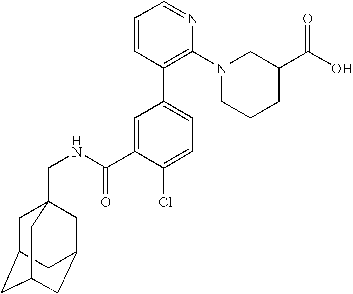

a) 1-(3-Bromo-2-pyridinyl)-3-piperidinecarboxylic acid, ethyl ester

Prepared according to the method of Example 59 (a) using 2,3-dibromopyridine (250 mg) and ethyl nipecotate (250 mg) to give the sub-title compound as an oil (325 mg).

MS: APCI(+ve) 313/315 (M+H⁺).

b) 1-[3-[4-Chloro-3-[[(tricyclo[3.3.1.1^{3,7}]dec-1-ylmethyl)amino]carbonyl]phenyl]-2-pyridinyl]-3-piperidinecarboxylic acid, ethyl ester

Prepared according to the method of Example 59 (b) using [4-chloro-3-[[(tricyclo[3.3.1.1^{3,7}]dec-1-ylmethyl)amino]carbonyl]phenyl]-boronic acid (Example 2 (a)) (300 mg) and 1-(3-bromo-2-pyridinyl)-3-piperidinecarboxylic acid, ethyl ester (Example 61 (a)) (320 mg) to give the sub-title compound as a foam (70 mg).

MS: APCI(+ve) 536/538 (M+H⁺).

c) 1-[3-[4-Chloro-3-[[(tricyclo[3.3.1.1^{3,7}]dec-1-ylmethyl)amino]carbonyl]phenyl]-2-pyridinyl]-3-piperidinecarboxylic acid

Prepared according to the method of Example 59 (c) using 1-[3-[4-chloro-3-[[(tricyclo[3.3.1.1^{3,7}]dec-1-ylmethyl)amino]carbonyl]phenyl]-2-pyridinyl]-4-piperidinecarboxylic acid, ethyl ester (Example 61 (b)) (70 mg) to give the title compound as a solid (35 mg).

MS: APCI(+ve) 508/510 (M+H⁺).

m.p. 125-130° C.

¹H NMR (400 MHz, d₆-DMSO) δ 8.37 (1H, t), 8.19 (1H, dd), 7.72 (1H, dd), 7.60 (1H, d), 7.57 (1H, dd), 7.54 (1H, d), 6.99 (1H, dd), 3.37 (2H, d), 2.95 (2H, d), 2.64 (2H, t), 1.94 (3H, s), 1.67 (3H, d), 1.60 (3H, d), 1.53 (6H, s), 1.38-1.80 (m, 5H).

### EXAMPLE 62

### 1-[3-[4-Chloro-3-[[(tricyclo[3.3.1.1^{3,7}]dec-1-ylmethyl)amino]carbonyl]phenyl]-2-pyridinyl]-3-azetidinecarboxylic acid

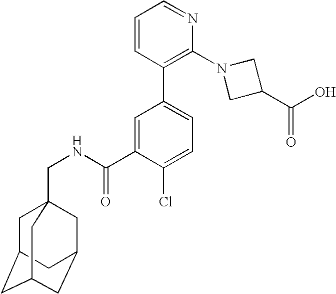

a) 1-(3-Bromo-2-pyridinyl)-3-azetidinol

To a solution of 2,3-dibromopyridine (700 mg) in pyridine (1 mL) at reflux was added, portionwise over 8 hours, azetidinol hydrochloride (1.4 g) and the mixture heated at reflux for a further 16 hours. The mixture was poured into water and the solid removed by filtration. The aqueous was then concentrated to dryness in vacuo and the residue purified (Varian C-18 cartridge, water:methanol gradient as eluant) to give the sub-title compound as a solid (145 mg).

MS: APCI(+ve) 229/231 (M+H⁺).

b) 1-(3-Bromo-2-pyridinyl)-3-azetidinol, methanesulfonate ester

To a solution of 1-(3-bromo-2-pyridinyl)-3-azetidinol (Example 62 (a)) (145 mg) and triethylamine (0.1 mL) in dichloromethane (10 mL) was added dropwise a solution of methanesulfonyl chloride (0.07 mL) and the reaction mixture stirred at room temperature for 2 hours. The mixture was washed with dilute acetic acid and aqueous sodium bicarbonate solution. The organics were dried over magnesium sulphate, filtered, concentrated to dryness in vacuo and purified (SiO₂, dichloromethane as eluant) to give the sub-title compound as a colourless oil (194 mg).

MS: APCI(+ve) 307/309 (M+H⁺).

c) 1-(3-Bromo-2-pyridinyl)-3-azetidinecarbonitrile

A mixture of 1-(3-bromo-2-pyridinyl)-3-azetidinol, methanesulfonate ester (Example 62 (b)) (194 mg) and sodium cyanide (388 mg) in N,N-dimethylformamide (2 mL) was heated at 110° C. for 6 days. The reaction mixture was partitioned between water and dichloromethane. The organics were collected and purified by chromatography (SiO₂, dichloromethane as eluant) to give the sub-title compound as a colourless oil (115 mg).

MS: APCI(+ve) 238/240 (M+H⁺).

d) 1-(3-Bromo-2-pyridinyl)-3-azetidinecarboxylic acid

A mixture of 1-(3-bromo-2-pyridinyl)-3-azetidinecarbonitrile (Example 62 (c)) (150 mg) and potassium hydroxide (150 mg) in ethanol (2 mL) and water (2 mL) was heated to 100° C. by microwave irradiation. After 30 minutes, the reaction was cooled and concentrated to dryness in vacuo. The residue was dissolved in water (5 mL) and extracted with dichloromethane (5×5 mL), the combined organic fractions were dried over magnesium sulphate, filtered and concentrated to dryness in vacuo to give the sub-title compound as an oil (150 mg).

MS: APCI(+ve) 257/259 (M+H⁺).

e) 1-(3-Bromo-2-pyridinyl)-3-azetidinecarboxylic acid, methyl ester

To a solution of 1-(3-bromo-2-pyridinyl)-3-azetidinecarboxylic acid (Example 62 (d)) (150 mg) in dichloromethane (2 mL) containing N,N-dimethylformamide (1 drop), was added oxalyl chloride (0.5 mL) and the mixture was stirred at room temperature for 30 minutes. The reaction mixture was concentrated to dryness and the residue dissolved in methanol (5 mL). The solution was stirred at room temperature for 30 minutes and then concentrated to dryness and the residue purified (Varian C-18 cartridge, water:methanol gradient as eluant) to give the sub-title compound as an oil (76 mg).

MS: APCI(+ve) 271/273 (M+H⁺).

f) 1-[3-[4-Chloro-3-[[(tricyclo[3.3.1.1^{3,7}]dec-1-ylmethyl)amino]carbonyl]phenyl]-2-pyridinyl]-3-azetidinecarboxylic acid, methyl ester

Prepared according to the method of Example 59 (b) using [4-chloro-3-[[(tricyclo[3.3.1.1^{3,7}]dec-1-ylmethyl)amino]carbonyl]phenyl]-boronic acid (Example 2 (a)) (100 mg) and 1-(3-bromo-2-pyridinyl)-3-azetidinecarboxylic acid, methyl ester (Example 62 (e)) (74 mg) in tetrahydrofuran (2 mL) and water (2 mL) to give the sub-title compound as a foam (107 mg).

MS: APCI(+ve) 494/496 (M+H⁺).

g) 1-[3-[4-Chloro-3-[[(tricyclo[3.3.1.1^{3,7}]dec-1-ylmethyl)amino]carbonyl]phenyl]-2-pyridinyl]-3-azetidinecarboxylic acid

Prepared according to the method of Example 59 (c) using 1-[3-[4-chloro-3-[[(tricyclo[3.3.1.1^{3,7}]dec-1-ylmethyl)amino]carbonyl]phenyl]-2-pyridinyl]-3-azetidinecarboxylic acid, methyl ester (Example 62 (f)) (105 mg) to give the title compound as a solid (76 mg).

MS: APCI(+ve) 480 (M+H⁺).

m.p. 145-150° C.

¹H NMR (400 MHz, d₆-DMSO) δ 8.36 (1H, t), 8.17 (1H, s), 7.33-7.56 (4H, m), 6.87 (1H, t), 3.78 (2H, t), 3.68 (2H, t), 3.26-3.39 (1H, m), 2.96 (2H, d), 1.94 (3H, s), 1.67 (3H, d), 1.60 (3H, d), 1.53 (6H, s).

### EXAMPLE 63

### 3-[4-Chloro-3-[[(tricyclo[3.3.1.1^{3,7}]dec-1-ylmethyl)amino]carbonyl]phenyl]-6-methyl-2-pyridinecarboxylic acid

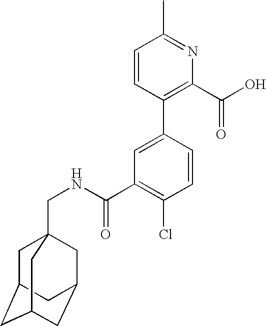

a) 2-Chloro-5-(2-chloro-6-methyl-3-pyridinyl)-N-(tricyclo[3.3.1.1^{3,7}]dec-1-ylmethyl)-benzamide

A mixture of [4-chloro-3-[[(tricyclo[3.3.1.1^{3,7}]dec-1-ylmethyl)amino]carbonyl]phenyl]-boronic acid (Example 2 (a)) (174 mg), 3-bromo-2-chloro-6-methyl-pyridine (104 mg), potassium carbonate (138 mg) and bis(triphenylphosphine)palladium(II) chloride (27 mg) in tetrahydrofuran (2 mL) and water (2 mL) was stirred at room temperature under a nitrogen atmosphere over 72 hours. The mixture was filtered through diatomaceous earth, washing with methanol, and the filtrate was concentrated in vacuo. Purification by chromatography (SiO₂, isohexane:ethyl acetate 1:1 as eluant) gave the sub-title compound as a solid (135 mg).

MS: APCI(+ve) 429/431 (M+H⁺)

b) 2-Chloro-5-(2-cyano-6-methyl-3-pyridinyl)-N-(tricyclo[3.3.1.1^{3,7}]dec-1-ylmethyl)-benzamide

A mixture of 2-chloro-5-(2-chloro-6-methyl-3-pyridinyl)-N-(tricyclo[3.3.1.1^{3,7}]dec-1-ylmethyl)-benzamide (Example 63 (a)) (0.13 g), to(dibenzylideneacetone)palladium (0.05 g), 1,1′-bis(diphenylphosphino)ferrocene (0.11 g) and copper (I) cyanide (0.13 g) in 1,4-dioxane (3 mL) was heated at reflux under nitrogen for 3 hours. The reaction was passed through a plug of silica, eluting with acetonitrile and the liquors concentrated to dryness in vacuo. The residue was then purified by chromatography (SiO₂, 1:2 ethyl acetate:isohexane as eluant) to give the sub-title compound as a solid (0.10 g)

MS: APCI(+ve) 420/422 (M+H⁺).

c) 3-[4-Chloro-3-[[(tricyclo[3.3.1.1^{3,7}]dec-1-ylmethyl)amino]carbonyl]phenyl]-6-methyl-2-pyridinecarboxylic acid

A solution of 2-chloro-5-(2-cyano-6-methyl-3-pyridinyl)-N-(tricyclo[3.3.1.1^{3,7}]dec-1-ylmethyl)-benzamide (Example 63 (b)) (70 mg) in acetonitrile (2 mL) was treated with a solution of potassium hydroxide (0.25 g) in water (0.6 mL) and the mixture was heated in a microwave at 120° C. for 60 minutes. The reaction mixture was then concentrated to dryness in vacuo, dissolved in water (5 ml) and concentrated hydrochloric acid (5 mL) then heated at reflux for 24 hours. The reaction mixture was concentrated to dryness and purified (Varian C-18 cartridge, water methanol gradient as eluant, then by Varian NH₂ cartridge using methanol (100 mL) and then 5% acetic acid in methanol (100 mL) as eluant) to give the title compound as a solid (25 mg).

MS: APCI(+ve) 439 (M+H⁺).

m.p. 125-130° C.

¹H NMR (400 MHz, d₆-DMSO) δ 8.38 (1H, t), 7.75 (1H, d), 7.73-7.66 (1H, m), 7.50-7.47 (1H, m), 7.44 (1H, s), 7.39 (1H, d), 2.94 (2H, d), 2.07 (3H, s), 1.93 (3H, s), 1.70-1.56 (6H, m), 1.52 (6H, s).

### EXAMPLE 64

### 3-[4-Chloro-3-[[(tricyclo[3.3.1.1^{3,7}]dec-1-ylmethyl)amino]carbonyl]phenyl]-5-methyl-2-pyridinecarboxylic acid

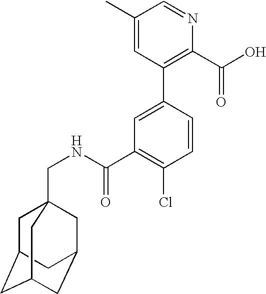

a) 3-Bromo-5-methyl-2-pyridinamine

To a solution of 2-amino-3-picoline (10.8 g) in glacial acetic acid (30 mL) at 80° C. was added dropwise a solution of bromine (5.5 mL) in glacial acetic acid (5.5 mL) and the temperature maintained for 1 hour. The reaction was concentrated to dryness and the residue dissolved in water (100 mL) which was then basified with 0.880 ammonia solution and extracted into dichloromethane. The organics were dried (MgSO₄), filtered and concentrated to dryness. The resulting residue was recrystallised from isohexane/ethyl acetate to give the sub-title compound as a solid (18.3 g).

MS: APCI(+ve) 187/189 (M+H⁺).

b) 3-Bromo-2-chloro-5-methyl-pyridine

To a solution of 3-bromo-5-methyl-2-pyridinamine (Example 64 (a)) (1.0 g) in a mixture of concentrated hydrochloric acid (5 mL) and water (3 mL) at 0° C. was added a solution of sodium nitrite (0.36 g) in water (3 mL). After the addition was complete, the reaction mixture was neutralised by the addition of 0.880 ammonia and the resulting precipitate collected by filtration and purified by chromatography (SiO₂, dichloromethane as eluant) to give the sub-title compound as a solid (0.47 g)

MS: APCI(+ve) 206 (M+H⁺).

c) 2-Chloro-5-(2-chloro-5-methyl-3-pyridinyl)-N-(tricyclo[3.3.1.1^{3,7}]dec-1-ylmethyl)-benzamide

Prepared according to the method of Example 63 (a) using [4-chloro-3-[[(tricyclo[3.3.1.1^{3,7}]dec-1-ylmethyl)amino]carbonyl]phenyl]-boronic acid (Example 2 (a)) (113 mg), 3-bromo-2-chloro-5-methyl-pyridine (Example 64 (b)) (209 mg) and tetrakis(triphenylphosphine)palladium(0) (35 mg) at 50° C. to give the sub-title compound as a solid (220 mg).

MS: APCI(+ve) 429/431 (M+H⁺).

d) 2-Chloro-5-(2-cyano-5-methyl-3-pyridinyl)-N-(tricyclo[3.3.1.1^{3,7}]dec-1-ylmethyl)-benzamide

Prepared according to the method of Example 63 (b) using 2-chloro-5-(2-chloro-5-methyl-3-pyridinyl)-N-(tricyclo[3.3.1.1^{3,7}]dec-1-ylmethyl)-benzamide (Example 64 (c)) (0.15 g) to give the sub-title compound as a solid (0.13 g).

MS: APCI(+ve) 420/422 (M+H⁺).

e) 3-[4-Chloro-3-[[(tricyclo[3.3.1.1^{3,7}]dec-1-ylmethyl)amino]carbonyl]phenyl]-5-methyl-2-pyridinecarboxylic acid

Prepared according to the method of Example 21(c) using 2-chloro-5-(2-cyano-5-methyl-3-pyridinyl)-N-(tricyclo[3.3.1.1^{3,7}]dec-1-ylmethyl)-benzamide (Example 64 (d)) (0.12 g) to give the title compound as a solid (80 mg).

MS: APCI(+ve) 439 (M+H⁺).

m.p. 125-130° C.

¹H NMR (300 MHz, CDCl₃) δ 8.44 (1H, s), 7.63 (1H, s), 7.59 (1H, s), 7.44 (1H, d), 7.35 (1H, d), 6.38 (1H, s), 3.16 (2H, d), 2.47 (3H, s), 2.00 (3H, s), 1.73 (3H, d), 1.64 (3H, d), 1.59 (6H, s).

### EXAMPLE 65

### 1-[3-[4-Chloro-3-[[(tricyclo[3.3.1.1^{3,7}]dec-1-ylmethyl)amino]carbonyl]phenyl]-2-pyridinyl]-4-hydroxy-4-piperidinecarboxylic acid

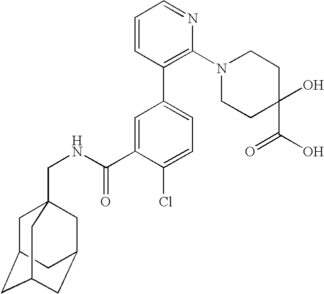

a) 1-(3-Bromo-2-pyridinyl)-4-hydroxy-4-piperidinecarboxylic acid, methyl ester

Prepared according to the method of Example 59 (a) using 2,3-dibromopyridine (145 mg) and 4-hydroxy-4-piperidinecarboxylic acid methyl ester (97 mg) to give the sub-title compound as an oil (44 mg).

MS: APCI(+ve) 315/317 (M+H⁺).

b) 1-[3-[4-Chloro-3-[[(tricyclo[3.3.1.1^{3,7}]dec-1-ylmethyl)amino]carbonyl]phenyl]-2-pyridinyl]-4-hydroxy-4-piperidinecarboxylic acid

Prepared according to the method of Example 59 (b) using [4-chloro-3-[[(tricyclo[3.3.1.1^{3,7}]dec-1-ylmethyl)amino]carbonyl]phenyl]-boronic acid (Example 2 (a)) (55 mg) and 1-(3-bromo-2-pyridinyl)-4-hydroxy-4-piperidinecarboxylic acid, methyl ester (Example 65 (a)) (44 mg) in tetrahydrofuran (4 mL) and water (2 mL). Upon completion of the reaction potassium hydroxide (100 mg) in water (1 ml) was added and the mixture stirred at room temperature overnight. The reaction mixture was concentrated to dryness and the residue purified (Varian C-18 cartridge, water:methanol gradient as eluant) to give the title compound as a white solid (50 mg).

MS: APCI(+ve) 522 (M+H⁺).

m.p. 145-150° C.

1H NMR (300 MHz, CDCL3) δ 8.27 (1H, d), 8.21 (1H, d), 7.60 (1H, dd), 7.51 (1H, dd), 7.49 (1H, d), 6.96 (1H, dd), 6.49 (1H, t), 3.37 (2H, d), 3.22-3.11 (2H, m), 3.20 (2H, d), 2.14-1.96 (4H, m), 2.01 (3H, s), 1.74 (3H, d), 1.64 (3H, d), 1.59 (6H, s).

### EXAMPLE 66

### 1-[3-[4-Chloro-3-[[(tricyclo[3.3.1.1^{3,7}]dec-1-ylmethyl)amino]carbonyl]phenyl]-5-fluoro-2-pyridinyl]-4-piperidinecarboxylic acid

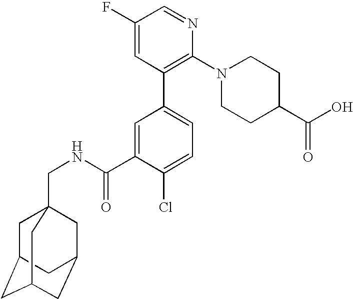

a) 3-Bromo-5-fluoro-2-pyridinamine

Prepared according to the method of Example 64 (a) using 5-fluoro-2-pyridinamine (2.0 g) to give the sub-title compound as a solid (2.2 g).

MS: APCI(+ve) 191/193 (M+H⁺).

b) 2,3-Dibromo-5-fluoro-pyridine

To a solution of 3-bromo-5-fluoro-2-pyridinamine (Example 66 (a)) (1.0 g) in hydrobromic acid (2.5 mL) at 0° C. was added bromine (0.85 mL) dropwise, maintaining the temperature below 5° C. Then a solution of sodium nitrite (0.92 g) in water (2 mL) was added dropwise again at below 5° C. The mixture was then stirred at 0° C. for 30 minutes before being treated dropwise with a solution of sodium hydroxide (2.0 g) in water (2 mL). The reaction mixture was allowed to warm to room temperature and was partitioned between water and ethyl acetate, the organics were washed with water, dried (MgSO₄), filtered and concentrated to dryness to give an orange oil which was purified by chromatography (SiO₂ cartridge eluting with dichloromethane) to give the sub-title compound as a solid (1.1 g).

¹H NMR (300 MHz, CDCl₃) δ 7.71 (1H, dd), 8.26 (1H, d).

c) 1-(3-Bromo-5-fluoro-2-pyridinyl)-4-piperidinecarboxylic acid, methyl ester

Prepared according to the method of Example 59 (a) using 2,3-dibromo-5-fluoro-pyridine (Example 66 (b)) (250 mg) and methyl isonipecotate (250 mg) to give the sub-title compound as an oil (125 mg).

MS: APCI(+ve) 317/319 (M+H⁺).

d) 1-[3-[4-Chloro-3-[[(tricyclo[3.3.1.1^{3,7}]dec-1-ylmethyl)amino]carbonyl]phenyl]-5-fluoro-2-pyridinyl]-4-piperidinecarboxylic acid, methyl ester

Prepared according to the method of Example 26(a) using [4-chloro-3-[[(tricyclo[3.3.1.1^{3,7}]dec-1-ylmethyl)amino]carbonyl]phenyl]-boronic acid (Example 2 (a)) (146 mg) and 1-(3-bromo-5-fluoro-2-pyridinyl)-4-piperidinecarboxylic acid, methyl ester (Example 66 (c)) (120 mg) to give the sub-title compound as a solid (150 mg).

MS: APCI(+ve) 540/542 (M+H⁺).

e) 1-[3-[4-Chloro-3-[[(tricyclo[3.3.1.1^{3,7}]dec-1-ylmethyl)amino]carbonyl]phenyl]-5-fluoro-2-pyridinyl]-4-piperidinecarboxylic acid

To a suspension of 1-[3-[4-chloro-3-[[(tricyclo[3.3.1.1^{3,7}]dec-1-ylmethyl)amino]carbonyl]phenyl]-5-fluoro-2-pyridinyl]-4-piperidinecarboxylic acid, methyl ester (Example 66 (d)) (150 mg) in methanol (8 mL) was added a solution of sodium hydroxide (200 mg) in water (1.5 mL). The resulting mixture was then stirred at room temperature over 72 hours. The reaction was acidified with acetic acid, concentrated to dryness and purified (Varian NH₂ cartridge using acetonitrile and then 50% acetic acid in acetonitrile as eluant) to give the title compound as a solid (114 mg).

MS: APCI(+ve) 526 (M+H⁺).

m.p. 130-135° C.

1H NMR (300 MHz, d₆-DMSO) δ 8.34 (1H, t), 8.22 (1H, d), 7.77 (1H, d), 7.73 (1H, s), 7.64 (1H, dd), 7.58 (1H, d), 3.28 (2H, d), 2.95 (2H, d), 2.65 (2H, t), 2.27 (1H, t), 1.95 (3H, s), 1.68 (3H, d), 1.59 (3H, d), 1.53 (6H, s), 1.47-1.77 (4H, m).

### EXAMPLE 67

### 4′-Methyl-3′-[[(tricyclo[3.3.1.1^{3,7}]dec-1-ylmethyl)amino]carbonyl]-[1,1′-biphenyl]-2-carboxylic acid

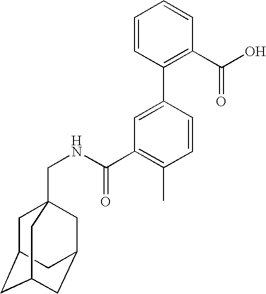

2-Methyl-5-(4,4,5,5-tetramethyl-1,3,2-dioxaborolan-2-yl)-N-(tricyclo[3.3.1.1^{3,7}]dec-1-ylmethyl)-benzamide (Example 55 (b)) (150 mg), 2-bromo-benzoic acid methyl ester (79 mg), sodium carbonate (116 mg), tetrakis(triphenylphosphine)palladium(0) (15 mg), tetrahydrofuran (2 mL) and water (1 mL) were heated in a microwave at 120° C. for 30 minutes. 48% Sodium hydroxide solution (300 μL) was added and the mixture was heated for 30 minutes at 120° C. in a microwave. The reaction was neutralised with acetic acid and purification by RP-HPLC (acetonitrile:aqueous trifluoroacetic acid, Symmetry) gave the title compound (58 mg).

MS: APCI(+ve) 404 (M+H⁺).

m.p. 85-92° C.

¹H NMR (300 MHz, d₆-DMSO) δ 8.16 (1H, t), 7.74-7.70 (1H, m), 7.62-7.55 (1H, m), 7.49-7.41 (2H, m), 7.33-7.23 (3H, m), 2.95 (2H, d), 2.38 (3H, s), 1.94 (3H, s), 1.71-1.55 (6H, m), 1.51 (6H, s).

### EXAMPLE 68

### 1-[3-[4-Methyl-3-[[(tricyclo[3.3.1.1^{3,7}]dec-1-ylmethyl)amino]carbonyl]phenyl]-2-pyridinyl]-4-piperidinecarboxylic acid

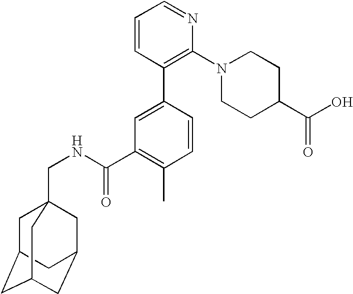

a) 1-(3-Bromo-2-pyridinyl)-4-piperidinecarboxylic acid methyl ester

2,3-Dibromopyridine (3.0 g) and methyl isonipecotate (5.4 g) were heated in a microwave at 130° C. for 30 minutes. Purification by chromatography (SiO₂, dichloromethane as eluant) gave the sub-title compound as a colourless oil (2.4 g).

MS: APCI(+ve) 299/301 (M+H⁺).

¹H NMR (400 MHz, d₆-DMSO) δ 8.24 (1H, dd), 7.95 (1H, dd), 6.92 (1H, dd), 3.63 (3H, s), 3.62-3.59 (2H, m), 2.84 (2H, dd), 2.60-2.52 (1H, m), 1.97-1.89 (2H, m), 1.77-1.66 (2H, m).

b) 1-[3-[4-Methyl-3-[[(tricyclo[3.3.1.1^{3,7}]dec-1-ylmethyl)amino]carbonyl]phenyl]-2-pyridinyl]-4-piperidinecarboxylic acid

2-Methyl-5-(4,4,5,5-tetramethyl-1,3,2-dioxaborolan-2-yl)-N-(tricyclo[3.3.1.1^{3,7}]dec-1-ylmethyl)-benzamide (Example 55 (b)) (200 mg), l-(3-bromo-2-pyridinyl)-4-piperidinecarboxylic acid methyl ester (Example 68 (a)) (146 mg), sodium carbonate (155 mg), tetrakis(triphenylphosphine)palladium(0) (15 mg), tetrahydrofuran (2 mL) and water (1 mL) were heated in a microwave at 120° C. for 60 minutes. 48% Sodium hydroxide solution (300 μL) and methanol (2 mL) were added and the mixture stirred for 12 hours. The reaction was neutralised with acetic acid and purified by RP-HPLC, (acetonitrile:aqueous trifluoroacetic acid, Symmetry) to give the title compound (77 mg).

MS: APCI(+ve) 488 (M+H⁺).

m.p. 60° C.

¹H NMR (300 MHz, d₆-DMSO) δ 8.21-8.12 (2H, m), 7.73 (1H, d), 7.57 (2H, d), 7.33 (1H, d), 7.10 (1H, dd), 3.43 (2H, d), 2.96 (2H, d), 2.75 (2H, t), 2.38 (3H, s), 2.36-2.26 (1H, m), 1.95 (3H, s), 1.79-1.42 (16H, m).

### EXAMPLE 69

### 6-[4-Methyl-3-[[(tricyclo[3.3.1.1^{3,7}]dec-1-ylmethyl)amino]carbonyl]phenyl]-2-pyridinecarboxylic acid

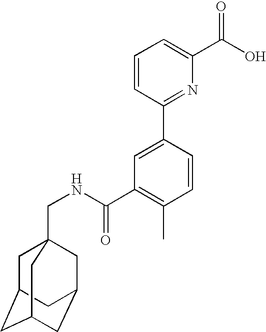

2-Methyl-5-(4,4,5,5-tetramethyl-1,3,2-dioxaborolan-2-yl)-N-(tricyclo[3.3.1.1^{3,7}]dec-1-ylmethyl)-benzamide (Example 55 (b)) (200 mg), 6-chloro-2-pyridinecarboxylic acid, methyl ester (108 mg), sodium carbonate (207 mg), tetrakis(triphenylphosphine)palladium (0) (15 mg), tetrahydrofuran (2 mL) and water (1 mL) were heated in a microwave at 120° C. for 60 minutes. 48% Sodium hydroxide solution (300 μL) and methanol (2 mL) were added and the mixture was heated for 30 minutes at 90° C. in a microwave. The reaction was neutralised with acetic acid and purified by RP-HPLC, (acetonitrile:aqueous trifluoroacetic acid, Symmetry) to give the title compound (44 mg).

MS: APCI(+ve) 405 (M+H⁺).

m.p. 227° C.

¹H NMR (400 MHz, d₆-DMSO) δ 13.20 (1H, s), 8.26 (1H, t), 8.21 (1H, dd), 8.14-8.10 (2H, m), 8.07 (1H, t), 7.98 (1H, dd), 7.39 (1H, d), 2.99 (2H, d), 2.41 (3H, s), 1.95 (3H, s), 1.71-1.59 (6H, m), 1.56-1.52 (6H, m).

### EXAMPLE 70

### 4-[4-Chloro-3-[[(tricyclo[3.3.1.1^{3,7}]dec-1-ylmethyl)amino]carbonyl]phenyl]-3-pyridinecarboxylic acid

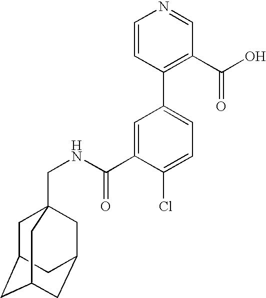

A mixture of [4-chloro-3-[[(tricyclo[3.3.1.1^{3,7}]dec-1-ylmethyl)amino]carbonyl]phenyl]-boronic acid (Example 2 (a)) (200 mg), 4-chloronicotinic acid (91 mg), potassium carbonate (159 mg) and tetrakis(triphenylphosphine)palladium(0) (67 mg) in tetrahydrofuran (0.5 mL) and water (0.5 mL) were heated in a microwave at 120° C. for 2 hours. The products were filtered through diatomaceous earth, washing with methanol (2×10 mL), the filtrate was concentrated and the residue purified by RP-HPLC (acetonitrile:aqueous trifluoroacetic acid, Symmetry) to give the title compound as a solid (7 mg).

MS: APCI(+ve) 425 (M+H⁺).

m.p. 104-107° C.

¹H NMR (300 MHz, d₆-DMSO) δ 8.95 (1H, s), 8.77 (1H, d), 8.41 (1H, t), 7.58 (1H, d), 7.51-7.42 (3H, m), 2.95 (2H, d), 1.94 (3H, s), 1.72-1.56 (6H, m), 1.53 (6H, s).

### EXAMPLE 71

### 6-[4-Chloro-3-[[(tricyclo[3.3.1.1^{3,7}]dec-1-ylmethyl)amino]carbonyl]phenyl]-3-methyl-2-pyridinecarboxylic acid

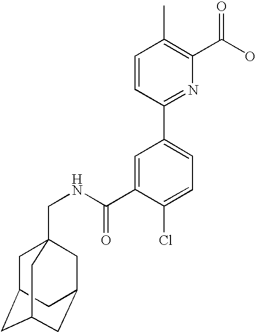

a) 3-Methyl-2-pyridinecarboxylic acid 1-oxide, methyl ester

A solution of 3-methyl 2-pyridinecarboxylic acid, methyl ester (340 mg) in acetic acid (5 mL) and 35% aqueous hydrogen peroxide (5 mL) was heated at 60° C. for 5 hours before being stirred at room temperature overnight. The reaction mixture was quenched by pouring into a sodium sulfite/ice water mixture (50 mL) and then extracted into dichloromethane (3×25 mL). The organics were dried over magnesium sulphate, filtered and concentrated to dryness to give the sub-title compound as a colourless oil (300 mg).

MS: APCI(+ve) 168 (M+H⁺).

b) 6-Chloro-3-methyl-2-pyridinecarboxylic acid, methyl ester

A solution of 3-methyl-2-pyridinecarboxylic acid 1-oxide, methyl ester (Example 71 (a)) (300 mg) in phosphorous oxychloride (1 mL) was heated at 60° C. for 16 hours. The reaction mixture was then poured into water (10 mL) and extracted with dichloromethane (2×10 mL). The organics were dried over magnesium sulphate, filtered and concentrated to dryness to give the sub-title compound as a colourless oil (300 mg).

MS: APCI(+ve) 185/187 (M+H⁺).

c) 6-[4-Chloro-3-[[(tricyclo[3.3.1.1^{3,7}]dec-1-ylmethyl)amino]carbonyl]phenyl]-3-methyl-2-pyridinecarboxylic acid

A mixture of [4-chloro-3-[[(tricyclo[3.3.1.1^{3,7}]dec-1-ylmethyl)amino]carbonyl]phenyl]-boronic acid (Example 2 (a)) (190 mg), 6-chloro-3-methyl-2-pyridinecarboxylic acid, methyl ester (Example 71(b)) (200 mg), potassium carbonate (150 mg) and dichlorobis(triphenylphosphine) palladium (II) (30 mg) in tetrahydrofuran (7 mL) and water (7 mL) were heated at reflux for 5 hours, sodium hydroxide (100 mg) was added and the mixture was stirred for 16 hours at room temperature. The products were concentrated and purified (Varian C-18 cartridge, water:acetonitrile gradient as eluant, followed by Varian NH₂ resin, acetonitrile:acetic acid gradient as eluant) to give the title compound as a solid (60 mg).

MS: APCI(+ve) 439 (M+H⁺).

m.p. 180-185° C.

¹H NMR (400 MHz, d₆-DMSO) δ 8.43 (1H, t), 8.19-8.15 (2H, m), 8.08 (1H, d), 7.86 (1H, d), 7.60 (1H, d), 2.98 (2H, d), 2.47 (3H, s), 1.95 (3H, s), 1.68 (3H, d), 1.61 (3H, d), 1.55 (6H, s).

### EXAMPLE 72

### 6-[4-Chloro-3-[[(tricyclo[3.3.1.1^{3,7}]dec-1-ylmethyl)amino]carbonyl]phenyl]-4-(trifluoromethyl)-2-pyridinecarboxylic acid

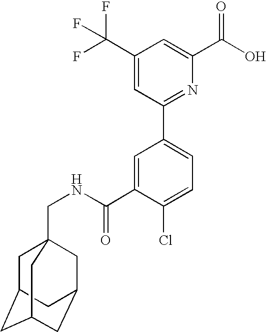

a) 6-Chloro-4-(trifluoromethyl)-2-pyridinecarboxylic acid, methyl ester

To a solution of 6-chloro-4-trifluoromethoxy-pyridine-2-carboxylic acid (144 mg) in dichloromethane (5 mL) containing 1 drop of N,N-dimethylformamide was added dropwise oxalyl chloride (0.2 mL). The mixture was stirred at room temperature for 1 hour and then concentrated to dryness in vacuo, azeotroping with dichloromethane (×3). The residue was dissolved in methanol and the solution stirred at room temperature for 1 hour then concentrated to dryness in vacuo to give the sub-title compound as a white solid (152 mg).

¹H NMR (300 MHz, CDCL₃) δ 8.27 (1H, s), 7.76 (1H, s), 4.05 (3H, s).

b) 6-[4-Chloro-3-[[(tricyclo[3.3.1.1^{3,7}]dec-1-ylmethyl)amino]carbonyl]phenyl]-4-(trifluoromethyl)-2-pyridinecarboxylic acid

Prepared according to the method of Example 71 (c) using [4-chloro-3-[[(tricyclo[3.3.1.1^{3,7}]dec-1-ylmethyl)amino]carbonyl]phenyl]-boronic acid (Example 2 (a)) (230 mg), 6-chloro-4-(trifluoromethyl)-2-pyridinecarboxylic acid, methyl ester (Example 72 (a)) (152 mg), potassium carbonate (170 mg) and dichlorobis(triphenylphosphine) palladium (II) (20 mg) at 50° C. to give the title compound as a solid (50 mg).

MS: APCI(+ve) 493 (M+H⁺).

m.p. 220-225° C.

¹H NMR (300 MHz, d₆-DMSO) δ 8.69 (1H, s), 8.46 (1H, t), 8.39-8.36 (2H, m), 8.21 (1H, s), 7.68 (1H, t), 2.99 (2H, d), 1.96 (3H, s), 1.69 (3H, d), 1.61 (3H, d), 1.57 (6H, s).

### EXAMPLE 73

### 5-[6-(Acetylamino)-2-methyl-3-pyridinyl]-2-chloro-N-(tricyclo[3.3.1.1^{3,7}]dec-1-ylmethyl)-benzamide

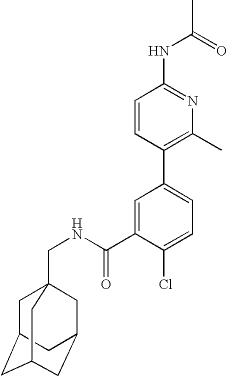

a) N-(5-Bromo-6-methyl-2-pyridinyl)-acetamide

To a solution of 5-bromo-6-methyl-2-pyridinamine (500 mg) in dichloromethane (10 mL) was added triethylamine (0.42 mL) followed by acetyl chloride (0.25 mL). The reaction was heated at reflux for 1 hour then cooled and washed with water. The organics were dried over magnesium sulphate, filtered and concentrated to dryness in vacuo and the residue was triturated with isohexane to give the sub-title compound as a solid (520 mg).

MS: APCI(+ve) 229/231 (M+H⁺).

b) 5-[6-(Acetylamino)-2-methyl-3-pyridinyl]-2-chloro-N-(tricyclo[3.3.1.1^{3,7}]dec-1-ylmethyl)-benzamide

A mixture of [4-chloro-3-[[(tricyclo[3.3.1.1^{3,7}]dec-1-ylmethyl)amino]carbonyl]phenyl]-boronic acid (Example 2 (a)) (350 mg), N-(5-bromo-6-methyl-2-pyridinyl)-acetamide (Example 73 (a)) (229 mg), potassium carbonate (280 mg) and

dichlorobis(triphenylphosphine)palladium (II) (50 mg) in toluene (10 mL)/water (1 mL) and ethanol (1 mL) was heated at 80° C. under a nitrogen atmosphere for 3 hours. The products were filtered through diatomaceous earth, washing with dichloromethane. The solvent was removed in vacuo and the residue was purified by chromatography (SiO₂, 50% to 60% ethyl acetate in isohexane as eluant) to give the title compound as a solid (100 mg).

MS: APCI(+ve) 452 (M+H⁺).

m.p. 172-177° C.

¹H NMR (400 MHz, d₆-DMSO) δ 8.07 (1H, d), 7.98 (1H, s), 7.66 (1H, d), 7.53 (1H, d), 7.47 (1H, d), 7.30 (1H, dd), 6.31 (1H, t), 3.20 (2H, d), 2.40 (3H, s), 2.22 (3H, s), 2.02 (3H, s), 1.74 (3H, d), 1.65 (3H, d), 1.57-1.61 (6H, m).

Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37 (3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing concentrations of test compound typically from 30 μM-0.001 μM. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure>5.5. For example, the following table shows the pIC₅₀ figures for a representative selection of compounds:

| Compound of | |
|---|---|
| Example No. | pIC₅₀ |
| 1 | 6.5 |
| 11 | 6.8 |

1. A compound of formula (I), or a pharmaceutically acceptable salt or solvate thereof, 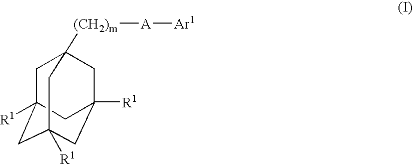
wherein m represents 1, 2 or 3;
each R¹ independently represents a hydrogen atom or a halogen;
A represents C(O)NH or NHC(O);
Ar¹ represents a group 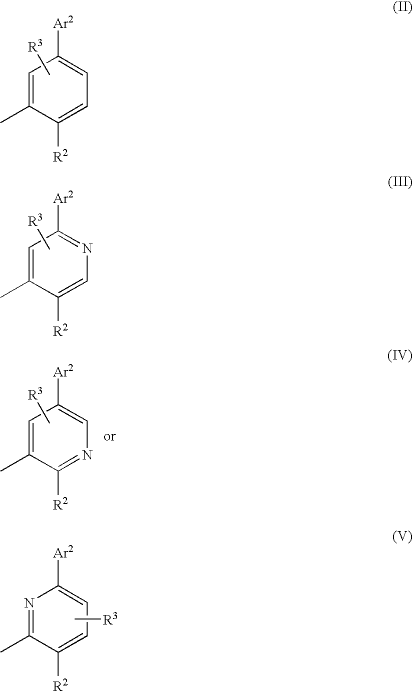
one of R² and R³ represents halogen, nitro, NR⁴R⁵, hydroxyl, or a group selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;
R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁-C₆ alkylCO₂R⁷, C₁₋₆ alkylsulphonylaminocarbonyl, NHR⁸, R⁹, XR¹⁰, C(O)NHOH and NR²⁹R²⁹; and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;
R⁶ and R⁷ each independently represent a hydrogen atom of a C₁-C₆ alkyl group;
R⁸ represents CN, C₁₋C₆ alkylsulphonyl, C₁₋C₆ alkylcarbonyl, C₁₋C₆ alkoxycarbonyl, C₁₋C₆ alkylaminosulphonyl, or (di)-C₁₋C₆ alkylaminosulphonyl;
R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;
M represents a bond, oxygen, S(O)_{q} or NR²³;
X represents oxygen, S(O)ₛ, NR²⁴, C₁₋C₆ alkylene, O(CH₂)₁₋₆, NR²⁵(CH₂)₁₋₆, or S(O)ₜ(CH₂)₁₋₆;
p, q, s and t each independently represent 0, 1 or 2;
R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁₋C₆alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and
R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
provided that:
when m is 1 and Ar¹ is a group (II) and Ar² is phenyl substituted by XR¹⁰ in a position para to Ar¹ and X is CH₂, then R¹⁰ is not a 2,4-dioxothiazolyl group, and
when m is 1 and Ar¹ is a group (II) and Ar² is phenyl substituted by MC₁₋C₆ alkylCO₂R⁷ in a position para to Ar¹, then M does not represent a bond.

2. A compound according to claim 1, wherein A represents NHC(O).

3. A compound according to claim 1, wherein Ar² represents phenyl, thienyl or a 5- or 6-membered heteroaromatic ring comprising from 1 to 2 nitrogen atoms.

4. A compound according to claim 1, wherein Ar² is substituted by at least one substituent selected from carboxyl, -C₁₋C₆alkylCO₂H, —OC₁₋C₆alkylCO₂H, —N(C₁₋₄alkyl)C₁₋C₆alkylCO₂H, —NHCN, —NHSO₂C₁₋C₆alkyl, tetrazolyl and —OC₁₋C₆ alkyltetrazolyl, and wherein Ar² can further be optionally substituted by at least one substituent selected from halogen and C₁₋C₆ alkyl.

5. A compound according to claim 1, wherein Ar¹ represents a group 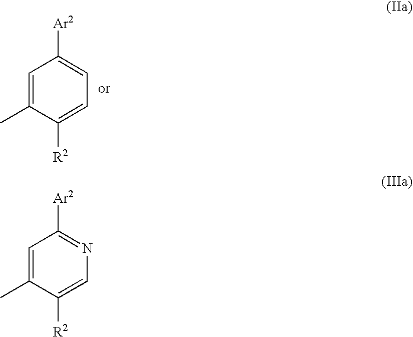
wherein R² represents halogen, nitro, NH₂, hydroxyl, or a C₁-C₆ alkyl optionally substituted by at least one halogen.

6. A compound according to claim 1 which is selected from:
4′-Chloro-3′-[[(tricyclo[3.3.1.1^{3,7}]dec-1-ylmethyl)amino]carbonyl]-[1,1′-biphenyl]-4-carboxylic acid,
4′-Chloro-3′-[[(tricyclo[3.3.1.1^{3,7}]dec-1-ylmethyl)amino]carbonyl]-[1,1′-biphenyl]-3-carboxylic acid,
4′-Chloro-3′-[[(tricyclo[3.3.1.1^{3,7}]dec-1-ylmethyl)amino]carbonyl]-[1,1′-biphenyl]-2-carboxylic acid,
2-Chloro-5-[6-(cyanoamino)pyrazinyl]-N-(tricyclo[3.3.1.1^{3,7}]dec-1-ylmethyl)-benzamide,
2-Chloro-5-[3-(cyanamino)pyrazinyl]-N-(tricyclo[3.3.1.1^{3,7}]dec-1-ylmethyl)-benzamide,
3-[4-Chloro-3-[[(tricyclo[3.3.1.1^{3,7}]dec-1-ylmethyl)amino]carbonyl]phenyl]-2-pyrazinecarboxylic acid,
3-[5-Chloro-4-[[(tricyclo [3.3.1.1^{3,7}]dec-1-ylmethyl)amino]carbonyl]-2-pyridinyl]-benzoic acid,
2-Chloro-5-[3-[(methylsulfonyl)amino)pyrazinyl]-N-(tricyclo[3.3.1.1^{3,7}]dec-1-ylmethyl)-benzamide,
2-Chloro-5-[3-(1-H-tetrazol-5-yl)pyrazinyl]-N-(tricyclo[3.3.1.1^{3,7}]dec-1-ylmethyl)-benzamide,
2-[4-Chloro-3-[[(tricyclo[3.3.1.1^{3,7}]dec-1-ylmethyl)amino]carbonyl]phenyl]-3-pyridinecarboxylic acid,
5-[4-Chloro-3-[[(tricyclo[3.3.1.1^{3,7}]dec-1-ylmethyl)amino]carbonyl]phenyl]-3-pyridinecarboxylic acid,
2-[4-Chloro-3-[[(tricyclo[3.3.1.1^{3,7}]dec-1-ylmethyl)amino]carbonyl]phenyl]-4-pyridinecarboxylic acid,
2-[4-Chloro-3-[[(tricyclo[3.3.1.1^{3,7}]dec-1-ylmethyl)amino]carbonyl]phenyl]-6-methyl-3-pyridinecarboxylic acid,
(2S)-2-[[4′-chloro-3′-[[(tricyclo[3.3.1.1^{3,7}]dec-1-ylmethyl)amino]carbonyl][1,1′-biphenyl]-2-yl]oxy]-propanoic acid,
[[4′-Chloro-3′-[[(tricyclo[3.3.1.1^{3,7}]dec-1-ylmethyl)amino]carbonyl][1,1′-biphenyl]-2-yl]oxy]-acetic acid,
3-[[4′-Chloro-3′-[[(tricyclo[3.3.1.1^{3,7}]dec-1-ylmethyl)amino]carbonyl][1,1′-biphenyl]-2-yl]oxy]-propanoic acid,
5-Chloro-2-[4-chloro-3-[[(tricyclo[3.3.1.1^{3,7}]dec-1-ylmethyl)amino]carbonyl]phenyl]-3-pyridinecarboxylic acid,
4′-Chloro-6-methyl-3′-[[(tricyclo[3.3.1.1^{3,7}]dec-1-ylmethyl)amino]carbonyl]-[1,1′-biphenyl]-2-carboxylic acid,
3-[4-Chloro-3-[[(tricyclo[3.3.1.1^{3,7}]dec-1-ylmethyl)amino]carbonyl]phenyl]-2-thiophenecarboxylic acid,
6-[4-Chloro-3-[[(tricyclo[3.3.1.1^{3,7}]dec-1-ylmethyl)amino]carbonyl]phenyl]-2-pyridinecarboxylic acid,
3-[4-Chloro-3-[[(tricyclo[3.3.1.1^{3,7}]dec-1-ylmethyl)amino]carbonyl]phenyl]-2-pyridinecarboxylic acid,
2-Chloro-5-[2-(1H-tetrazol-5-yl)-3-pyridinyl]-N-(tricyclo[3.3.1.1^{3,7}]dec-1-ylmethyl)-benzamide,
2-[4-Chloro-3-[[(tricyclo[3.3.1.1^{3,7}]dec-1-ylmethyl)amino]carbonyl]phenyl]-4-oxazolecarboxylic acid,
4′-Chloro-4-methyl-3′-[[(tricyclo[3.3.1.1^{3,7}]dec-1-ylmethyl)amino]carbonyl]-[1,1′-biphenyl]-2-carboxylic acid,
6-[4-Chloro-3-[[(tricyclo[3.3.1.1^{3,7}]dec-1-ylmethyl)amino]carbonyl]phenyl]-N-(methylsulfonyl)-2-pyridinecarboxamide,
N-[3-[4-Chloro-3-[[(tricyclo[3.3.1.1^{3,7}]dec-1-ylmethyl)amino]carbonyl]phenyl]-2-pyridinyl]-glycine,
2-Chloro-5-[6-[(methylsulfonyl)amino]-2-pyridinyl]-N-(tricyclo[3.3.1.1^{3,7}]dec-1-ylmethyl)-benzamide,
[[3-[4-Chloro-3-[[(tricyclo[3.3.1.1^{3,7}]dec-1-ylmethyl)amino]carbonyl]phenyl]-2-pyridinyl]oxy]-acetic acid,
2-Chloro-5-[3-(1H-tetrazol-5-ylmethoxy)-2-pyridinyl]-N-(tricyclo[3.3.1.1^{3,7}]dec-1-ylmethyl)-benzamide,
4′-Chloro-4-methoxy-3′-[[(tricyclo[3.3.1.1^{3,7}]dec-1-ylmethyl)amino]carbonyl]-[1,1′-biphenyl]-2-carboxylic acid,
4-[4-Chloro-3-[[(tricyclo [3.3.1.1^{3,7}]dec-1-ylmethyl)amino]carbonyl]phenyl]-1-methyl-1H-pyrazole-3-carboxylic acid,
4-[4-Chloro-3-[[(tricyclo[3.3.1.1^{3,7}]dec-1-ylmethyl)amino]carbonyl]phenyl]-1-methyl-1H-pyrazole-5-carboxylic acid,
N-[3-[4-Chloro-3-[[(tricyclo[3.3.1.1^{3,7}]dec-1-ylmethyl)amino]carbonyl]phenyl]pyrazinyl]-N-methyl-glycine,
1-[3-[4-Chloro-3-[[(tricyclo[3.3.1.1^{3,7}]dec-1-ylmethyl)amino]carbonyl]phenyl]pyrazinyl]-4-piperidinecarboxylic acid,
4′-Chloro-6-fluoro-3′-[[(tricyclo[3.3.1.1^{3,7}]dec-1-ylmethyl)amino]carbonyl]-[1,1′-biphenyl]-2-carboxylic acid,
4′-Chloro-5-fluoro-3′-[[(tricyclo[3.3.1.1^{3,7}]dec-1-ylmethyl)amino]carbonyl]-[1,1′-biphenyl]-2-carboxylic acid,
4′-Chloro-3′-[[(tricyclo[3.3.1.1^{3,7}]dec-1-ylmethyl)amino]carbonyl]-[1,1′-biphenyl]-2-acetic acid,
[[4′-Chloro-3′-[[(tricyclo[3.3.1.1^{3,7}]dec-1-ylmethyl)amino]carbonyl][1,1′-biphenyl]-3-yl]oxy]-acetic acid,
(2R)-2-[[4′-Chloro-3′-[[(tricyclo[3.3.1.1^{3,7}]dec-1-ylmethyl)amino]carbonyl][1,1′-biphenyl]-2-yl]oxy]-propanoic acid,
[[4′-Chloro-3′-[[(tricyclo[3.3.1.1^{3,7}]dec-1-ylmethyl)amino]carbonyl][1,1′-biphenyl]-4-yl]oxy]-acetic acid,
(2S)-2-[[4′-Chloro-3′-[[(tricyclo[3.3.1.1^{3,7}]dec-1-ylmethyl)amino]carbonyl][1,1-biphenyl]-3-yl]oxy ]-propanoic acid,
4,4′-Dichloro-3′-[[(tricyclo[3.3.1.1^{3,7}]dec-1-ylmethyl)amino]carbonyl]-[1,1′-biphenyl]-2-carboxylic acid,
(2S)-2-[[4′-Chloro-3′-[[(tricyclo[3.3.1.1^{3,7}]dec-1-ylmethyl)amino]carbonyl][1,1′-biphenyl]-4-yl]oxy]-propanoic acid,
3-Chloro-6-[4-chloro-3-[[(tricyclo[3.3.1.1^{3,7}]dec-1-ylmethyl)amino]carbonyl]phenyl]-2-pyridinecarboxylic acid,
3-[4-Chloro-3-[[(tricyclo[3.3.1.1^{3,7}]dec-1-ylmethyl)amino]carbonyl]phenyl]-4-pyridinecarboxylic acid,
[[2-[4-Chloro-3-[[(tricyclo[3.3.1.1^{3,7}]dec-1-ylmethyl)amino]carbonyl]phenyl]-3-pyridinyl]oxy]-acetic acid,
N-[2-[4-Chloro-3-[[(tricyclo[3.3.1.1^{3,7}]dec-1-ylmethyl)amino]carbonyl]phenyl]-3-pyridinyl]-glycine,
4′-Chloro-4,5-difluoro-3′-[[(tricyclo[3.3.1.1^{3,7}]dec-1-ylmethyl)amino]carbonyl]-[1,1′-biphenyl]-2-carboxylic acid,
4′-Chloro-3′-[[(2-tricyclo[3.3.1.1^{3,7}]dec-1-ylethyl)amino]carbonyl]-[1,1′-biphenyl]-2-carboxylic acid,
3-[4-Chloro-3-[[(2-tricyclo[3.3.1.1^{3,7}]dec-1-ylethyl)amino]carbonyl]phenyl]-2-pyridinecarboxylic acid,
4′-Chloro-4-fluoro-3′-[[(tricyclo[3.3.1.1^{3,7}]dec-1-ylmethyl)amino]carbonyl]-[1,1′-biphenyl]-2-carboxylic acid,
2-[5-Chloro-4-[[(tricyclo[3.3.1.1^{3,7}]dec-1-ylmethyl)amino]carbonyl]-2-pyridinyl]-benzoic acid,
2-[4-Chloro-3-[[(tricyclo[3.3.1.1^{3,7}]dec-1-ylmethyl)amino]carbonyl]phenyl]-4-methyl-3-pyridinecarboxylic acid,
6-[4-Chloro-3-[[(tricyclo[3.3.1.1^{3,7}]dec-1-ylmethyl)amino]carbonyl]phenyl]-2-[(2-hydroxyethyl)methylamino]-3-pyridinecarboxylic acid,
3-[4-Methyl-3-[[(tricyclo[3.3.1.1^{3,7}]dec-1-ylmethyl)amino]carbonyl]phenyl]-2-pyridinecarboxylic acid,
4-[4-Chloro-3-[[(tricyclo[3.3.1.1^{3,7}]dec-1-ylmethyl)amino]carbonyl]phenyl]-1,3-dimethyl-1H-pyrazole-5-carboxylic acid,
2-[4-Methyl-3-[[(tricyclo[3.3.1.1^{3,7}]dec-1-ylmethyl)amino]carbonyl]phenyl]-3-pyridinecarboxylic acid,
3-[4-Chloro-3-[[(tricyclo[3.3.1.1^{3,7}]dec-1-ylmethyl)amino]carbonyl]phenyl]-2-pyridineacetic acid,
1-[3-[4-Chloro-3-[[(tricyclo[3.3.1.1^{3,7}]dec-1-ylmethyl)amino]carbonyl]phenyl]-2-pyridinyl]-4-piperidinecarboxylic acid,
1-[3-[4-Chloro-3-[[(tricyclo[3.3.1.1^{3,7}]dec-1-ylmethyl)amino]carbonyl]phenyl]-2-pyridinyl]-L-proline,
1-[3-[4-Chloro-3-[[(tricyclo[3.3.1.1^{3,7}]dec-1-ylmethyl)amino]carbonyl]phenyl]-2-pyridinyl]-3-piperidinecarboxylic acid,
1-[3-[4-Chloro-3-[[(tricyclo[3.3.1.1^{3,7}]dec-1-ylmethyl)amino]carbonyl]phenyl]-2-pyridinyl]-3-azetidinecarboxylic acid,
3-[4-Chloro-3-[[(tricyclo [3.3.1.1^{3,7}]dec-1-ylmethyl)amino]carbonyl]phenyl]-6-methyl-2-pyridinecarboxylic acid,
3-[4-Chloro-3-[[(tricyclo[3.3.1.1^{3,7}]dec-1-ylmethyl)amino]carbonyl]phenyl]-5-methyl-2-pyridinecarboxylic acid,
1-[3-[4-Chloro-3-[[(tricyclo[3.3.1.1^{3,7}]dec-1-ylmethyl)amino]carbonyl]phenyl]-2-pyridinyl]-4-hydroxy-4-piperidinecarboxylic acid,
1-[3-[4-Chloro-3-[[(tricyclo[3.3.1.1^{3,7}]dec-1-ylmethyl)amino]carbonyl]phenyl]-5-fluoro-2-pyridinyl]-4-piperidinecarboxylic acid,
4′-Methyl-3′-[[(tricyclo[3.3.1.1^{3,7}]dec-1-ylmethyl)amino]carbonyl]-[1,1′-biphenyl]-2-carboxylic acid,
1-[3-[4-Methyl-3-[[(tricyclo[3.3.1.1^{3,7}]dec-1-ylmethyl)amino]carbonyl]phenyl]-2-pyridinyl]-4-piperidinecarboxylic acid,
6-[4-Methyl-3-[[(tricyclo[3.3.1.1^{3,7}]dec-1-ylmethyl)amino]carbonyl]phenyl]-2-pyridinecarboxylic acid,
4-[4-Chloro-3-[[(tricyclo[3.3.1.1^{3,7}]dec-1-ylmethyl)amino]carbonyl]phenyl]-3-pyridinecarboxylic acid,
6-[4-Chloro-3-[[(tricyclo[3.3.1.1^{3,7}]dec-1-ylmethyl)amino]carbonyl]phenyl]-3-methyl-2-pyridinecarboxylic acid,
6-[4-Chloro-3-[[(tricyclo[3.3.1.1^{3,7}]dec-1-ylmethyl)amino]carbonyl]phenyl]-4-(trifluoromethyl)-2-pyridinecarboxylic acid, or
5-[6-(Acetylamino)-2-methyl-3-pyridinyl]-2-chloro-N-(tricyclo[3.3.1.1^{3,7}]dec-1-ylmethyl)-benzamide
or a pharmaceutically acceptable salt or solvate thereof.

7. A process for the preparation of a compound of formula (I) as defined in claim 1, or a pharmaceutically acceptable salt or solvate thereof which comprises:
(a) reacting a compound of formula 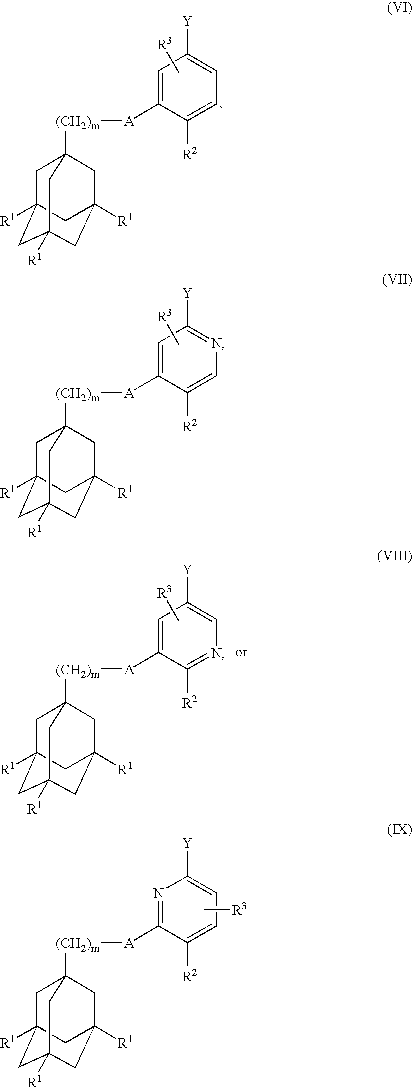
with a compound of formula
Z-Ar² (X)
wherein one of Y and Z represents a displaceable group such as a metallic, organometallic or organosilicon group and the other of Y and Z represents a leaving group such as a halogeno or sulphonyloxy group and R¹, m, A, Ar², R² and R³ are as defined for formula (I); or
(b) when Ar² is substituted by carboxyl, reacting a compound of formula (VI)-(IX) as defined in (a) above with a compound of formula
Z-Ar^{2a}—CN (XI)
wherein Z is as defined in formula (X), and Ar^{2a} represents phenyl or a 5- or 6-membered heteroaromatic ring comprising from 1 to 2 heteroatoms independently selected from nitrogen, oxygen and sulphur, followed by reaction with a base, then optionally followed by reaction with an acid; or
(c) when R⁹ represents tetrazolyl, reacting a compound of formula (VI)-(IX) as defined in (a) above with a compound of formula (XI) as defined in (b) above, followed by reaction with a suitable source of azide; or
(d) when R⁸ represents CN, C₁₋₆ alkylsulphonyl, C₁₋₆ alkylcarbonyl, C₁₋₆ alkoxycarbonyl, C₁₋₆ alkylaminosulphonyl, or (di)-C₁₋₆ alkylaminosulphonyl reacting a compound of formula (VI)-(IX) as defined in (a) above with a compound of formula
L¹-Ar^{2b}-Z (XII)
wherein L¹ represents a leaving group such as a halogeno or sulphonyloxy group, Ar^{2b} represents phenyl or a 5- or 6-membered heteroaromatic ring comprising from 1 to 2 heteroatoms independently selected from nitrogen, oxygen and sulphur, and Z is as defined in formula (X), followed by reaction with a compound of formula 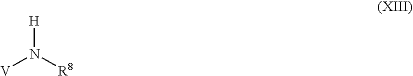
wherein V represents a hydrogen or a metallic group; or
(e) when Ar² is substituted by carboxyl, reacting a compound of formula (VI)-(IX) as defined in (a) above with a compound of formula (XII) as defined in (d) above, followed by reaction with a suitable source of cyanide, followed by reaction with a base, then optionally followed by reaction with an acid; or
(f) when R⁹ represents tetrazolyl, reacting a compound of formula (VI)-(IX) as defined in (a) above with a compound of formula (XII) as defined in (d) above, followed by reaction with a suitable source of cyanide, followed by reaction with a suitable source of azide; or
(g) when Ar² is substituted by carboxyl, reacting a compound of formula (VI)-(IX) as defined in (a) above with a compound of formula (XII) as defined in (d) above, followed by reaction with carbon monoxide and an alcohol in the presence of a suitable catalyst, followed by reaction with a base; or
(h) when Ar² represents a group of formula 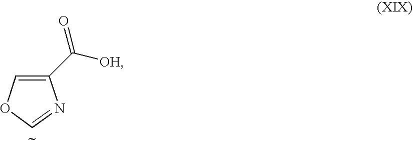
reacting a compound of formula 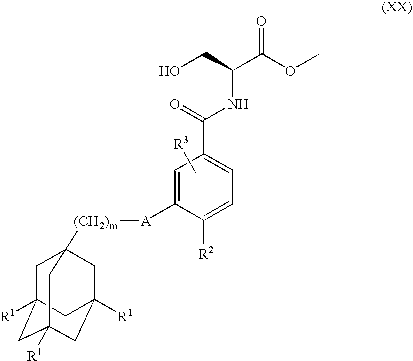
with a suitable cyclodehydrating reagent followed by reaction with a suitable oxidising reagent followed by reaction with a base; or
(i) when M represents oxygen or NR²³, reacting a compound of formula (VI)-(IX) as defined in (a) above, with a compound of formula (XII) as defined in (d) above, followed by reaction with a compound of formula
H-M-C₁₋₆alkyl-CO₂R⁷ (XXI)
wherein M represents oxygen or NR²³, and R²³ and R⁷ are as defined in formula (I), optionally followed by reaction with a suitable base or acid; or
(j) when M represents oxygen or NR²³, reacting a compound of formula (XXI) as defined in (i) above, with a compound, of formula (XII) as defined in (d) above, followed by reaction with a compound of formula (VI)-(IX) as defined in (a) above, optionally followed by reaction with a suitable base or acid; or
(k) when M represents oxygen or NR²³, reacting a compound of formula (VI)-(IX) as defined in (a) above, with a compound of formula
H-M-Ar^{2c}-Z (XXII)
wherein Ar^{2c} represents phenyl or a 5- or 6-membered heteroaromatic ring comprising from 1 to 2 heteroatoms independently selected from nitrogen, oxygen and sulphur, Z is as defined in formula (X), and M represents oxygen or NR²³, wherein R²³ is as defined in formula (I), followed by reaction with either β-propiolactone or a compound of formula
L¹-C₁₋₆alkyl-CO₂R⁷ (XXIII)
wherein R⁷ is as defined in formula (I), and L¹ is as defined in formula (XII), optionally followed by reaction with a suitable base or acid; or
(1) when X represents O(CH₂)₁₋₆ or NR²⁵(CH₂)₁₋₆ and R¹⁰ represents tetrazolyl, reacting a compound of formula (VI)-(IX) as defined in (a) above, with a compound of formula
H-M¹-Ar^{2d}-Z (XXIV),
wherein M¹ represents oxygen or NR²⁵, R²⁵ is as defined in formula (I), Ar^{2d} represents a phenyl or a 5- or 6-membered heteroaromatic ring comprising front 1 to 2 heteroatoms independently selected from nitrogen, oxygen and sulphur, and Z is as defined in formula (X), followed by reaction with a compound of formula
L¹-C₁₋₆alkyl-CN (XXV)
wherein L¹ is as defined in formula (XII), followed by reaction with a suitable source of azide; or
(m) when Ar² is substituted by carboxyl, reacting a compound of formula (VI)-(IX) as defined in (a) above with a compound of formula
Z-Ar^{2e}-C(O)H (XXVIII)
wherein Z is as defined in formula (X), and Ar^{2e} represents a phenyl or 5- or 6-membered heteroaromatic ring comprising from 1 to 2 heteroatoms independently selected from nitrogen, oxygen and sulphur, followed by reaction with a suitable oxidising agent; or
(n) reacting a compound of formula 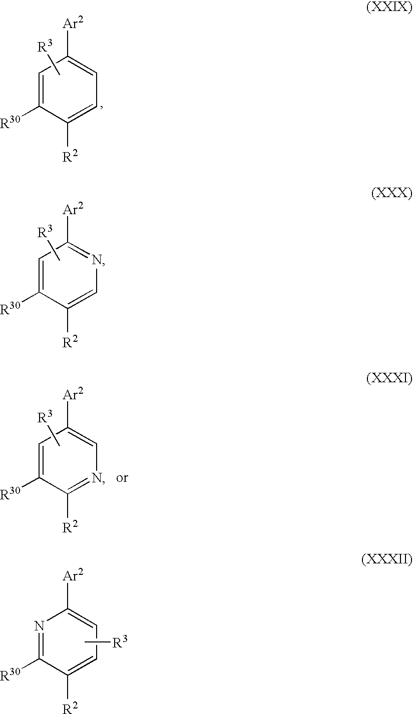
with a compound of formula 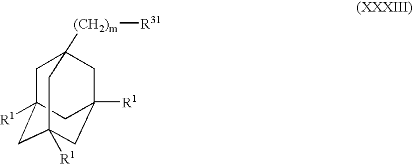
wherein one of R³⁰ and R³¹ represents NH₂ and the other of R³⁰ and R³¹ represents CO₂H, COBr or COCl, and Ar², R¹, R², R³, R⁶ and m are as defined in formula (I); or
(o) when R²⁸ and R²⁹ together with the nitrogen to which they are attached form a 3- to 8-membered saturated heterocyclic ring, which heterocyclic ring is substituted by CO₂R⁶, reacting a compound of formula (VI)-(IX) as defined in (a) above, with a compound of formula (XII) as defined in (d) above, followed by reaction with a compound of formula 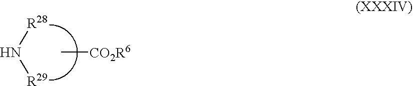
wherein R⁶, R²⁸ and R²⁹ are as defined in formula (I), optionally followed by reaction with a suitable base or suitable acid; or
(p) when R²⁸ and R²⁹ together with the nitrogen to which they are attached form a 3- to 8-membered saturated heterocyclic ring, which heterocyclic ring is substituted by CO₂R⁶, reacting a compound of formula (XII) as defined in (d) above with a compound of formula (XXXIV) as defined in (o) above, followed by reaction with a compound of formula (VI)-(IX) as defined in (a) above, optionally followed by reaction with a suitable base or acid;
and optionally after (a), (b), (c), (d), (e), (f), (g), (h), (i), (j), (k),(l), (m), (n), (o) or (p) carrying out one or more of the following:
converting the compound to a former compound of the invention
forming a pharmaceutically acceptable salt or solvate of the compound.

8. A pharmaceutical composition comprising a compound of formula (I), or a pharmaceutically acceptable salt or solvate thereof, 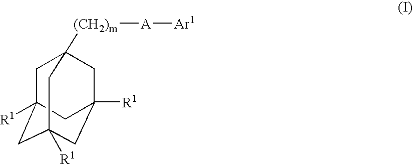
wherein m represents 1, 2 or 3;
each R¹ independently represents a hydrogen atom or a halogen;
A represents C(O)NH or NHC(O);
Ar¹ represents a group 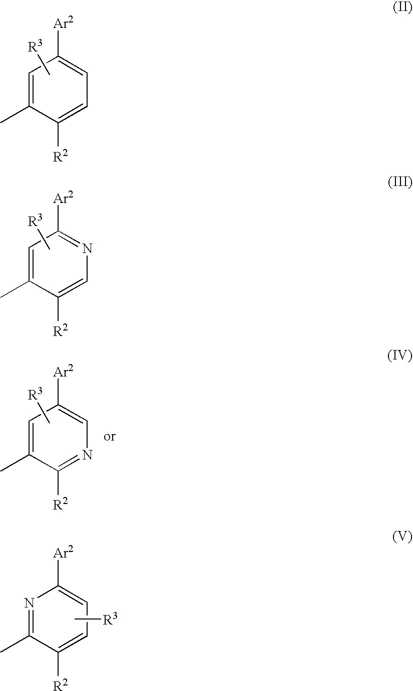
one of R² and R³ represents halogen, nitro, NR⁴R⁵, hydroxyl , or a group selected from (i) C₁-C₆ alkyl optionally substituted by at least one halogen and (ii) C₁-C₆ alkoxy optionally substituted by at least one halogen, and the other of R² and R³represents a hydrogen atom, halogen or a C₁-C₆ alkyl group optionally substituted by at least one halogen;
R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁₋₆ alkylCO₂R⁷, C₁₋₆ alkylsulphonylaminocarbonyl, NHR⁸, R⁹, XR¹⁰, C(O)NHOH and NR²⁸R²⁹;
and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;
R⁶ and R⁷ each independently represent a hydrogen atom or a C₁-C₆ alkyl group;
R⁸ represents CN, C₁₋C₆ alkylsulphonyl, C₁₋C₆ alkylcarbonyl, C₁₋C₆ alkoxycarbonyl, C₁₋C₆ alkylaminosulphonyl, or (di)-C₁₋C₆ alkylaminosulphonyl;
R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;
M represents a bond, oxygen, S(O)_{q} or NR²³;
X represents oxygen, S(O)ₛ, NR²⁴, C₁₋C₆ alkylene, O(CH₂)₁₋₆, NR²⁵(CH₂)₁₋₆, or S(O)ₜ(CH₂)₁₋₆;
p, q, s and t each independently represent 0, 1 or 2;
R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁₋₆ alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and
R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
provided that:
when m is 1 and Ar¹ is a group (II) and Ar² is phenyl substituted by XR¹⁰ in a position para to Ar¹ and X is CH₂, then R¹⁰ is not a 2,4-dioxothiazolyl group, and
when m is 1 and Ar¹ is a group (II) and Ar² is phenyl substituted by MC₁₋₆ alkylCO₂R⁷ in a position para to Ar¹, then M does not represent a bond
in association with a pharmaceutically acceptable adjuvant, diluent or carrier.

9. A process for the preparation of a pharmaceutical composition as claimed in claim 8 which comprises mixing a compound of formula (I), or a pharmaceutically acceptable salt or solvate thereof, with a pharmaceutically acceptable adjuvant, diluent or carrier.

10. (canceled)

11. A method of treating an inflammatory disorder in a subject, the method comprising administering to the subject a compound of formula (IA), or a pharmaceutically acceptable salt or solvate thereof 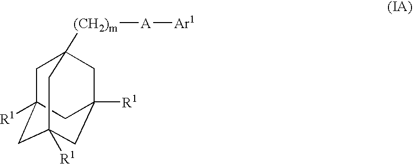
wherein m represents 1, 2 or 3;
each R¹ independently represents a hydrogen atom or a halogen;
A represents C(O)NH or NHC(O);
Ar¹ represents a group 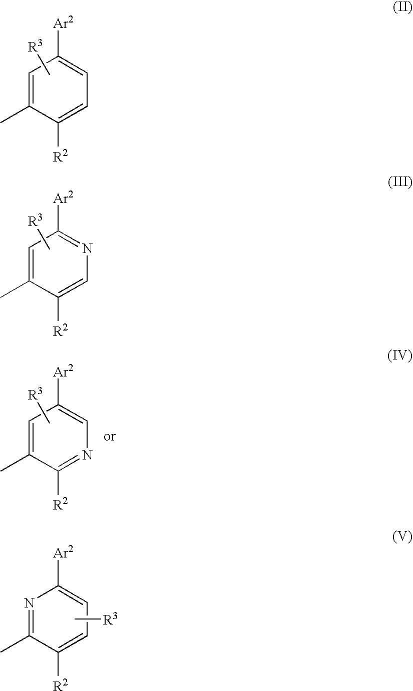
one of R² and R³ represents halogen, nitro, NR⁴R⁵, hydroxyl , or a group selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;
R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁-C₆ alkylCO₂R⁷, C₁₋₆ alkylsulphonylaminocarbonyl, NHR⁸, R⁹, XR¹⁰, C(O)NHOH and NR²⁸R²⁹;
and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;
R⁶ and R⁷ each independently represent a hydrogen atom or a C₁-C₆ alkyl group;
R⁸ represents CN, C₁-C₆ alkylsulphonyl, C₁-C₆ alkylcarbonyl, C₁-C₆ alkoxycarbonyl, C₁-C₆ alkylaminosulphonyl, or (di)-C₁-C₆ alkylaminosulphonyl;
R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;
M represents a bond, oxygen, S(O)_{q} or NR²³;
X represents oxygen, S(O)ₛ, NR²⁴, C₁-C₆ alkylene, O(CH₂)₁₋₆, NR²⁵(CH₂)₁₋₆, or S(O)ₜ(CH₂)₁₋₆;
p, q, s and t each independently represent 0, 1 or 2;
R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁-C₆ alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and
R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl.

12. The method according to claim 11, wherein the inflammatory disorder is rheumatoid arthritis.

13. The method according to claim 11, wherein the inflammatory disorder is osteoarthritis.

14. The method according to claim 11, wherein the inflammatory disorder is asthma or chronic obstructive pulmonary disease.

15. A method of treating atherosclerosis in a subject, the method comprising administering to the subject a compound of formula (IA), or a pharmaceutically acceptable salt or solvate thereof 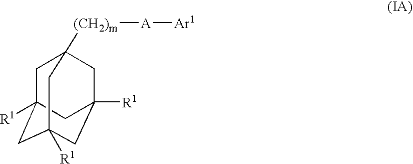
wherein m represents 1, 2 or 3;
each R¹ independently represents a hydrogen atom or a halogen;
A represents C(O)NH or NHC(O);
Ar¹ represents a group 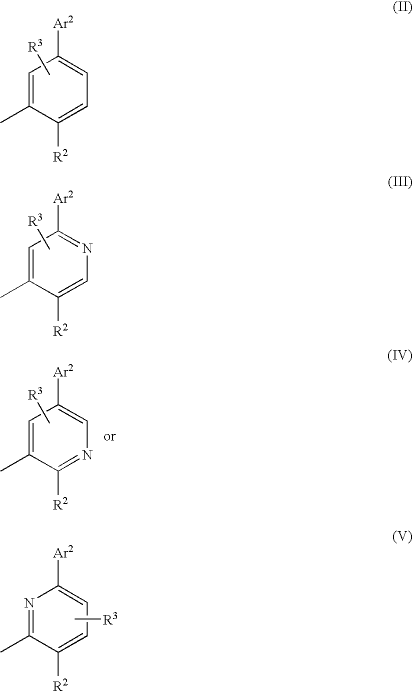
one of R² and R³ represents halogen, nitro, NR⁴R⁵, hydroxyl , or a group selected from (i) C₁-C₆ alkyl optionally substituted by at least one halogen and (ii) C₁-C₆ alkoxy optionally substituted by at least one halogen, and the other of R² and R³ represents a hydrogen atom, halogen or a C₁-C₆ alkyl group optionally substituted by at least one halogen;
R⁴ and R⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl;
Ar² represents phenyl or a 5- or 6-membered heteroaromatic ring comprising from 1 to 2 heteroatoms independently selected from nitrogen, oxygen and sulphur, which phenyl or heteroaromatic ring is substituted by at least one substituent selected from CO₂R⁶, MC₁-C₆ alkylCO₂R⁷, C₁₋₆ alkylsulphonylaminocarbonyl, NHR⁸, R⁹, XR¹⁰, C(O)NHOH and NR²⁸R²⁹;
and which phenyl or heteroaromatic ring can further be optionally substituted by at least one substituent selected from halogen, nitro, NR¹¹R¹², hydroxyl, S(O)ₚR¹³, a C₁-C₆ alkoxy group which C₁-C₆ alkoxy group can be optionally substituted by at least one halogen, and a C₁-C₆ alkyl group which C₁-C₆ alkyl group can be optionally substituted by at least one substituent selected from halogen, hydroxyl, NR¹⁴R¹⁵, SO₂NR¹⁶R¹⁷, NR¹⁸SO₂R¹⁹, NHCOR²⁰ and CONR²¹R²²;
R⁶ and R⁷ each independently represent a hydrogen atom or a C₁-C₆ alkyl group;
R⁸ represents CN, C₁-C₆ alkylsulphonyl, C₁-C₆ alkylcarbonyl, C₁-C₆ alkoxycarbonyl, C₁-C₆ alkylaminosulphonyl, or (di)-C₁-C₆ alkylaminosulphonyl;
R⁹ and R¹⁰ each independently represent tetrazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;
M represents a bond, oxygen, S(O)_{q} or NR²³;
X represents oxygen, S(O)ₛ, NR²⁴, C₁-C₆ alkylene, O(CH₂)₁₋₆, NR²⁵(CH₂)₁₋₆, or S(O)ₜ(CH₂)₁₋₆;
p, q, s and t each independently represent 0, 1 or 2;
R²⁸ and R²⁹ together with the nitrogen atom to which they are attached form a 3- to 8-membered saturated heterocyclic ring, which heterocyclic ring is substituted with at least one substituent independently selected from CO₂R⁶, MC₁-C₆ alkylCO₂R⁷, C₁₋₆ alkylsulphonylaminocarbonyl, C(O)NHOH, NHR⁸, R⁹ and XR¹⁰, and which 3- to 8-membered saturated heterocyclic ring can further be optionally substituted by at least one substituent independently selected from hydroxyl, halogen, C₁-C₆ alkoxy optionally substituted by at least one halogen, and a C₁-C₆ alkyl group which C₁-C₆ alkyl group can be optionally substituted by at least one substituent independently selected from halogen and hydroxyl; and
R¹¹, R¹², R¹³, R¹⁴, R¹⁵, R¹⁶, R¹⁷, R¹⁸, R¹⁹, R²⁰, R²¹, R²², R²³, R²⁴ and R²⁵ each independently represent a hydrogen atom or a group selected from C₁-C₆ alkyl and C₁-C₆ alkoxy, which C₁-C₆ alkyl or C₁-C₆ alkoxy group can be optionally substituted with at least one substituent selected from halogen and hydroxyl.
